SEQUENCE LISTING (70 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 3 - protein, 650 aa, Homo sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 4 - protein, 651 aa, Homo sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSAGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 5 - protein, 652 aa, Homo sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSAGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRQSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 6 - protein, 651 aa, Homo sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRQSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 7 - protein, 456 aa, Homo sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSAGPEDQPLTPTGSDPQSGE

SEQ ID NO: 8 - protein, 598 aa, Homo sapiens
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 9 - protein, 597 aa, Homo sapiens
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 10 - protein, 510 aa, Homo sapiens
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSSPAQLPTPRKKTSMLP

SEQ ID NO: 11 - protein, 482 aa, Homo sapiens
MTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 12 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 13 - protein, 246 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEAKPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 14 - protein, 154 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEAKQSSLPTIPIMGIVAGLVVLAAVVTGAAVAAVLWRKKSSD

SEQ ID NO: 15 - protein, 246 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRAKQSSLPTIPIMGIVAGLVVLAAVVTGAAVAAVLWRKKSSD

SEQ ID NO: 16 - protein, 319 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 17 - protein, 227 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEAKPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 18 - protein, 116 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASE

SEQ ID NO: 19 - protein, 5 aa, synthetic construct
  REGION 1..5: note = source = /note="Description of Artificial Sequence: Synthetic peptide"
SYDMS

SEQ ID NO: 20 - protein, 17 aa, synthetic construct
  REGION 1..17: note = source = /note="Description of Artificial Sequence: Synthetic peptide"
LISGGGSQTYYAESVKG

SEQ ID NO: 21 - protein, 11 aa, synthetic construct
  REGION 1..11: note = source = /note="Description of Artificial Sequence: Synthetic peptide"
PSGHYFYAMDV

SEQ ID NO: 22 - protein, 11 aa, synthetic construct
  REGION 1..11: note = source = /note="Description of Artificial Sequence: Synthetic peptide"
RASQGISNWLA

SEQ ID NO: 23 - protein, 7 aa, synthetic construct
  REGION 1..7: note = source = /note="Description of Artificial Sequence: Synthetic peptide"
AASSLQS

SEQ ID NO: 24 - protein, 9 aa, synthetic construct
  REGION 1..9: note = source = /note="Description of Artificial Sequence: Synthetic peptide"
QQAESFPHT

SEQ ID NO: 25 - protein, 120 aa, synthetic construct
  REGION 1..120: note = source = /note="Description of Artificial Sequence: Synthetic polypeptide"
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSS

SEQ ID NO: 26 - protein, 107 aa, synthetic construct
  REGION 1..107: note = source = /note="Description of Artificial Sequence: Synthetic polypeptide"
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIK

SEQ ID NO: 27 - protein, 472 aa, synthetic construct
  REGION 1..472: note = source = /note="Description of Artificial Sequence: Synthetic polypeptide"
MDMRVPAQLLGLLLLWLRGARCEVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 236 aa, synthetic construct
  REGION 1..236: note = source = /note="Description of Artificial Sequence: Synthetic polypeptide"
MDMRVPAQLLGLLLLWLRGARCDIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 329 aa, synthetic construct
  REGION 1..329: note = source = /note="Description of Artificial Sequence: Synthetic polypeptide"
ASTKGPSVFPLAPSSKSTSGGTAALGCLVKDYFPEPVTVSWNSGALTGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 107 aa, synthetic construct [sequence omitted]

SEQ ID NO: 31 - RNA, 18 nt, synthetic construct
  modified_base 17..18: mod_base = OTHER; note = thymine
  misc_feature 1..18: note = source = /note="Description of Artificial Sequence: Synthetic oligonucleotide"
  misc_feature 1..18: note = source = /note="Description of Combined DNA/RNA Molecule: Synthetic oligonucleotide"
  source 1..18: mol_type = other RNA
ggctgaacaaaggagatt

SEQ ID NO: 32 - DNA, 20 bp, synthetic construct
  misc_feature 1..20: note = source = /note="Description of Artificial Sequence: Synthetic primer"
  source 1..20: mol_type = other DNA
aacccacctgacttggatca

SEQ ID NO: 33 - DNA, 23 bp, synthetic construct
  misc_feature 1..23: note = source = /note="Description of Artificial Sequence: Synthetic primer"
  source 1..23: mol_type = other DNA
tgaggtatatgcaatcgtcaaaa

SEQ ID NO: 34 - DNA, 20 bp, synthetic construct
  misc_feature 1..20: note = source = /note="Description of Artificial Sequence: Synthetic primer"
  source 1..20: mol_type = other DNA
ctggaactggtggtgacaga

SEQ ID NO: 35 - DNA, 20 bp, synthetic construct
  misc_feature 1..20: note = source = /note="Description of Artificial Sequence: Synthetic primer"
  source 1..20: mol_type = other DNA
tctcctctaggtggtcagcc

SEQ ID NO: 36 - protein, 5 aa, synthetic construct
  REGION 1..5: note = source = /note="Description of Artificial Sequence: Synthetic peptide"
DYDMH

SEQ ID NO: 37 - protein, 16 aa, synthetic construct
  REGION 1..16: note = source = /note="Description of Artificial Sequence: Synthetic peptide"
TIDTAGDTYYPDSVRG

SEQ ID NO: 38 - protein, 9 aa, synthetic construct
  REGION 1..9: note = source = /note="Description of Artificial Sequence: Synthetic peptide"
DVPGTGFDH

SEQ ID NO: 39 - protein, 11 aa, synthetic construct
  REGION 1..11: note = source = /note="Description of Artificial Sequence: Synthetic peptide"
QGDSLRSYYES

SEQ ID NO: 40 - protein, 7 aa, synthetic construct
  REGION 1..7: note = source = /note="Description of Artificial Sequence: Synthetic peptide"
GKNNRPS

SEQ ID NO: 41 - protein, 11 aa, synthetic construct
  REGION 1..11: note = source = /note="Description of Artificial Sequence: Synthetic peptide"
NSRDNSGDHWV

SEQ ID NO: 42 - protein, 117 aa, synthetic construct
  REGION 1..117: note = source = /note="Description of Artificial Sequence: Synthetic polypeptide"
EVQLVESGGGLVQPGGSLRLSCAASGFTFSDYDMHWVRQATGKGLEWVATIDTAGDTYYPDSVRGRFTISRENAKNSLSLQMNSLRAGDTAVYFCARDVPGTGFDHWGQGTLVTVSS

SEQ ID NO: 43 - protein, 108 aa, synthetic construct
  REGION 1..108: note = source = /note="Description of Artificial Sequence: Synthetic polypeptide"
SSELTQDPAVSVALGQTVRITCQGDSLRSYYESWYQQKPGQAPVLVIYGKNNRPSGIPDRFSGSISGNTASLTITGTQAEDEADYYCNSRDNSGDHWVFGGGTRLTVL

SEQ ID NO: 44 - protein, 466 aa, synthetic construct
  REGION 1..466: note = source = /note="Description of Artificial Sequence: Synthetic polypeptide"
MDMRVPAQLLGLLLLWLRGARCEVQLVESGGGLVQPGGSLRLSCAASGFTFSDYDMHWVRQATGKGLEWVATIDTAGDTYYPDSVRGRFTISRENAKNSLSLQMNSLRAGDTAVYFCARDVPGTGFDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 236 aa, synthetic construct
  REGION 1..236: note = source = /note="Description of Artificial Sequence: Synthetic polypeptide"
MDMRVPAQLLGLLLLWLRGARCSSELTQDPAVSVALGQTVRITCQGDSLRSYYESWYQQKPGQAPVLVIYGKNNRPSGIPDRFSGSISGNTASLTITGTQAEDEADYYCNSRDNSGDHWVFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 46 - protein, 327 aa, synthetic construct
  REGION 1..327: note = source = /note="Description of Artificial Sequence: Synthetic polypeptide"
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 106 aa, synthetic construct
  REGION 1..106: note = source = /note="Description of Artificial Sequence: Synthetic polypeptide"
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 48 - protein, 5 aa, synthetic construct
  REGION 1..5: note = source = /note="Description of Artificial Sequence: Synthetic peptide"
SYAMH

SEQ ID NO: 49 - protein, 17 aa, synthetic construct
  REGION 1..17: note = source = /note="Description of Artificial Sequence: Synthetic peptide"
IIWFDGSQRYYADSVKG

SEQ ID NO: 50 - protein, 8 aa, synthetic construct
  REGION 1..8: note = source = /note="Description of Artificial Sequence: Synthetic peptide"
DELTGFDY

SEQ ID NO: 51 - protein, 11 aa, synthetic construct
  REGION 1..11: note = source = /note="Description of Artificial Sequence: Synthetic peptide"
RASQNIINFLN

SEQ ID NO: 52 - protein, 7 aa, synthetic construct
  REGION 1..7: note = source = /note="Description of Artificial Sequence: Synthetic peptide"
AASSLRS

SEQ ID NO: 53 - protein, 9 aa, synthetic construct
  REGION 1..9: note = source = /note="Description of Artificial Sequence: Synthetic peptide"
QQSFSTPLT

SEQ ID NO: 54 - protein, 117 aa, synthetic construct
  REGION 1..117: note = source = /note="Description of Artificial Sequence: Synthetic polypeptide"
QVQLVESGGGVVQPGRSLRLSCAASGFTFSSYAMHWVRQAPGKGLEWVAIIWFDGSQRYYADSVKGRFTISRDNSRDTLYLQMNSLRAEDTAVYYCARDELTGFDYWGQGTLVTVSS

SEQ ID NO: 55 - protein, 107 aa, synthetic construct
  REGION 1..107: note = source = /note="Description of Artificial Sequence: Synthetic polypeptide"
DIQMTQSPSSLSASVGDRVTISCRASQNIINFLNWYQQKPGKAPKVLIDAASSLRSGVPSRFSGSGSETDFSLTISSLQPEDFATYYCQQSFSTPLTFGGGTKVEIR

SEQ ID NO: 56 - protein, 466 aa, synthetic construct
  REGION 1..466: note = source = /note="Description of Artificial Sequence: Synthetic polypeptide"
MDMRVPAQLLGLLLLWLRGARCQVQLVESGGGVVQPGRSLRLSCAASGFTFSSYAMHWVRQAPGKGLEWVAIIWFDGSQRYYADSVKGRFTISRDNSRDTLYLQMNSLRAEDTAVYYCARDELTGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 236 aa, synthetic construct
  REGION 1..236: note = source = /note="Description of Artificial Sequence: Synthetic polypeptide"
MDMRVPAQLLGLLLLWLRGARCDIQMTQSPSSLSASVGDRVTISCRASQNIINFLNWYQQKPGKAPKVLIDAASSLRSGVPSRFSGSGSETDFSLTISSLQPEDFATYYCQQSFSTPLTFGGGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 5 aa, synthetic construct
  REGION 1..5: note = source = /note="Description of Artificial Sequence: Synthetic peptide"
DYDMH

SEQ ID NO: 59 - protein, 16 aa, synthetic construct
  REGION 1..16: note = source = /note="Description of Artificial Sequence: Synthetic peptide"
AIDTAGDTYYLGPVKG

SEQ ID NO: 60 - protein, 8 aa, synthetic construct
  REGION 1..8: note = source = /note="Description of Artificial Sequence: Synthetic peptide"
DSGSSFDY

SEQ ID NO: 61 - protein, 11 aa, synthetic construct
  REGION 1..11: note = source = /note="Description of Artificial Sequence: Synthetic peptide"
QGDSLRNYYES

SEQ ID NO: 62 - protein, 7 aa, synthetic construct
  REGION 1..7: note = source = /note="Description of Artificial Sequence: Synthetic peptide"
GKNNRPS

SEQ ID NO: 63 - protein, 11 aa, synthetic construct
  REGION 1..11: note = source = /note="Description of Artificial Sequence: Synthetic peptide"
MSRDSSGNHWV

SEQ ID NO: 64 - protein, 116 aa, synthetic construct
  REGION 1..116: note = source = /note="Description of Artificial Sequence: Synthetic polypeptide"
EVQLVESGGGLVQPGESLRLSCAASGFTFSDYDMHWVRQATGKGLEWVSAIDTAGDTYYLGPVKGRFTISRENAKNSLFLHMNSLRVGDTAVYYCIRDSGSSFDYWGQGTLVTVSS

SEQ ID NO: 65 - protein, 108 aa, synthetic construct
  REGION 1..108: note = source = /note="Description of Artificial Sequence: Synthetic polypeptide"
TSELTQDPAVSVALGQTVRITCQGDSLRNYYESWYQQKPGQAPVLVIYGKNNRPSGIPDRFSGSSSGNTASLIITGTQAEDEADYYCMSRDSSGNHWVFGGGTKVTVL

SEQ ID NO: 66 - protein, 465 aa, synthetic construct
  REGION 1..465: note = source = /note="Description of Artificial Sequence: Synthetic polypeptide"
MDMRVPAQLLGLLLLWLRGARCEVQLVESGGGLVQPGESLRLSCAASGFTFSDYDMHWVRQATGKGLEWVSAIDTAGDTYYLGPVKGRFTISRENAKNSLFLHMNSLRVGDTAVYYCIRDSGSSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 236 aa, synthetic construct
  REGION 1..236: note = source = /note="Description of Artificial Sequence: Synthetic polypeptide"
MDMRVPAQLLGLLLLWLRGARCTSELTQDPAVSVALGQTVRITCQGDSLRNYYESWYQQKPGQAPVLVIYGKNNRPSGIPDRFSGSSSGNTASLIITGTQAEDEADYYCMSRDSSGNHWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 68 - protein, 330 aa, Homo sapiens [sequence omitted]

SEQ ID NO: 69 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 70 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

(19) United States
(12) Patent Application Publication
(10) Pub. No.: US 2026/0217830 A1
(43) Pub. Date: Jul. 30, 2026

(54) COMBINATION OF LILRB1/2 PATHWAY INHIBITORS AND PD-1 PATHWAY INHIBITORS

(71) Applicant: AMGEN INC., Thousand Oaks, CA (US)

(72) Inventors: Xin YU, Foster City, CA (US); Wenjun OUYANG, Foster City, CA (US); Chi-Ming Kevin LI, Thousand Oaks, CA (US); Jackson Graeme EGEN, San Mateo, CA (US); Oh Kyu YOON, Pacifica, CA (US); Ian Halsey DRIVER, San Francisco, CA (US); Shunsuke TAKENAKA, Burnaby (CA); Christy Ann THOMSON, Port Moody (CA); Hongyu WANG, Coquitlam (CA)

(73) Assignee: AMGEN INC., Thousand Oaks, CA (US)

(21) Appl. No.: 19/643,606

(22) Filed: Apr. 9, 2026

Related U.S. Application Data
(62) Division of application No. 17/262,339, filed on Jul. 17, 2019, now Pat. No. 12,624,106.
(60) Provisional application No. 62/702,493, filed on Jul. 24, 2018.
(60) Provisional application No. 62/872,431, filed on Jul. 10, 2019.

(51) Int. Cl. C07K 16/28; A61K 39/00; A61P 35/00

(52) U.S. Cl. CPC C07K 16/2818; A61P 35/00; C07K 16/2803; C07K 16/2827; C07K 16/2833; A61K 2039/507; C07K 2317/34; C07K 2317/565; C07K 2317/73; C07K 2317/76; C07K 2317/92

(57) ABSTRACT

This invention relates to combination therapies comprising a Programmed Death 1 receptor (PD-1) pathway inhibitor, and a Leukocyte Immunoglobulin Like Receptor B (LILRB) signaling inhibitor, and the use of the combination therapies for the treatment of cancer. The invention also relates to the treatment of cancer patients who are refractory to monotherapy with a PD-1 pathway inhibitor.

### RELATED APPLICATIONS

This application is a divisional application of U.S. application Ser. No. 17/262,339, filed Jan. 22, 2021, which is the U.S. National Phase of International Application No. PCT/US2019/042234, filed Jul. 17, 2019 and published in English, which claims the benefit under 35 U.S.C. § 119 (e) to U.S. Provisional Application No. 62/702,493, filed Jul. 24, 2018, and U.S. Provisional Application No. 62/872,431, filed Jul. 10, 2019.

### SEQUENCE LISTING

The instant application contains a Sequence Listing which has been submitted in electronic format. The sequence listing is provided as a filed named A-2271-US04-DIV_SL, created Apr. 7, 2026, and is 96960 bytes in size. The information in the electronic format of the sequence listing is incorporated herein by reference in its entirety.

### FIELD OF THE INVENTION

The present invention relates to combination therapies useful for the treatment of cancer.

### BACKGROUND OF THE INVENTION

In the two-signal model T-cell activation is regulated by both positive and negative secondary co-stimulatory signals that function to maximize the host's protective immune responses, while maintaining immune tolerance and preventing autoimmunity. While the two-signal model was originally developed to describe the regulation of naive lymphocyte activation, a host's immune response is a multi-step and dynamic process and a similar paradigm of regulation by co-stimulatory signals applies to antigen-experienced T-cells. The positive and negative co-stimulatory signals regulating both naïve and antigen-experienced T cell activation are of therapeutic interest as manipulation of these signals provides a means to either enhance or terminate cell-based immune response. For example, the inhibitory receptor programmed cell death 1 (PD-1) is upregulated on chronically stimulated T cells and its sustained expression correlates with T cell dysfunction or anergy. As a result, therapeutics aimed at enhancing T cell activation by targeting of PD-1 or other molecules which signal through interactions with PD-1, such as programmed death ligand 1 (PD-L1) and programmed death ligand 2 (PD-L2), have generated great interest.

PD-L1 is overexpressed in many cancers and is often associated with poor prognosis (Okazaki T et al., Intern. Immun. 2007 19 (7): 813; Thompson R H et al., Cancer Res 2006, 66 (7): 3381). Interestingly, the majority of tumor infiltrating T lymphocytes predominantly express PD-1, in contrast to T lymphocytes in normal tissues and peripheral blood T lymphocytes, indicating that up-regulation of PD-1 on tumor-reactive T cells can contribute to impaired antitumor immune responses (Blood 2009 114 (8): 1537). This may be due to exploitation of PD-L1 signaling, mediated by PD-L1 expressing tumor cells interacting with PD-1 expressing T cells, resulting in attenuation of T cell activation and evasion of immune surveillance (Sharpe et al., Nat Rev 2002, Keir M E et al., 2008 Annu. Rev. Immunol. 26:677). Therefore, inhibition of the PD-L1/PD-1 interaction may enhance CD8+ T cell-mediated killing of tumors.

Therapeutic approach of blocking the PD-1/PD-L1 immune checkpoint proteins as anticancer agents has been successful in treating melanoma and NSCLC patients. However, overall more than 70% of cancer patients do not respond to PD-1 blockade. This may be because additional inhibitory mechanisms function to inhibit the activity of CD8+ T cells within the tumor microenvironment. For instance, CD8+ T cells can express other inhibitory receptors in addition to PD-1. Therefore, combination of therapeutic drugs that target different immune regulatory pathways may increase efficacy in cancer treatment. For example, clinical trial data of anti-PD-1 combined with anti-CTLA4, another checkpoint receptor on T cells, has shown improved efficacy in melanoma patients.

There is a need for improved therapies for the treatment of cancer, in particular in patients who are poor responders to anti-PD-1/anti-PD-L1 treatment.

### SUMMARY OF THE INVENTION

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).
E1. A method for increasing IFN-γ expression level in a subject that has cancer, comprising administering to the subject a therapeutically effective amount of (i) a first antibody, or antigen-binding fragment thereof, that binds PD-1, PD-L1, or PD-L2; and (ii) a second antibody, or antigen-binding fragment thereof, that binds LILRB1, LILRB2, or HLA-G.
E2. The method of E1, wherein said first antibody, or antigen-binding fragment thereof, binds PD-1.
E3. The method of E1 or E2, wherein said first antibody, or antigen-binding fragment thereof, binds human PD-1.
E4. The method of any one of E1-E3, wherein said first antibody is nivolumab, pembrolizumab, pidilizumab, or an antigen binding fragment thereof.
E5. The method of E1, wherein said first antibody, or antigen-binding fragment thereof, binds PD-L1, PD-L2, or both.
E6. The method of E1 or E5, wherein said first antibody, or antigen-binding fragment thereof, binds human PD-L1, PD-L2, or both.
E7. The method of any one of E1 and E5-E6, wherein said first antibody is avelumab, atezolizumab, durvalumab or an antigen binding fragment thereof.
E8. The method of any one of E1-E7, wherein said second antibody, or antigen-binding fragment thereof, binds LILRB1, LILRB2, or both.
E9. The method of any one of E1-E8, wherein said second antibody, or antigen-binding fragment thereof, binds human LILRB1, human LILRB2, or both.
E10. The method of any one of E1-E9, wherein said second antibody, or antigen-binding fragment thereof, binds both human LILRB1 and human LILRB2.
E11. The method of any one of E1-E7, wherein said second antibody, or antigen-binding fragment thereof, binds HLA-G.
E12. The method of any one of E1-E7 and E11, wherein said second antibody, or antigen-binding fragment thereof, binds human HLA-G.
E13. The method of any one of E1-E12, wherein said first antibody, or antigen-binding fragment thereof, and said second antibody, or antigen-binding fragment thereof, are administered concurrently.
E14. The method of any one of E1-E12, wherein said first antibody, or antigen-binding fragment thereof, and said second antibody, or antigen-binding fragment thereof, are administered sequentially.
E15. The method of E14, wherein one or more doses of said first antibody, or antigen-binding fragment thereof, are administered prior to administering said second antibody, or antigen-binding fragment thereof.
E16. The method of E14, wherein one or more doses of said second antibody, or antigen-binding fragment thereof, are administered prior to administering said first antibody, or antigen-binding fragment thereof.
E17. A method for increasing IFN-γ expression level in a subject that has cancer, comprising administering to the subject a therapeutically effective amount of (i) an antibody, or antigen-binding fragment thereof, that binds PD-1, PD-L1, or PD-L2; and (ii) a short interfering RNA (siRNA) that reduces the expression level of HLA-G, as compared to a control.
E18. The method of E17, wherein said siRNA comprises the nucleotide sequence of SEQ ID NO: 31.
E19. The method of any one of E1-E18, wherein said subject is a human.
E20. The method of any one of E1-E19, wherein said cancer is a solid tumor.
E21. The method of any one of E1-E20, wherein the cancer is brain cancer, bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), Squamous Cell Carcinoma of the Head and Neck (SCCHN), chronic myelogenous leukemia (CML), small lymphocytic lymphoma (SLL), malignant mesothelioma, colorectal cancer, or gastric cancer.
E22. The method of any one of E1-E21, wherein said subject is refractory to treatment with an anti-PD-1 antibody or antigen-binding fragment thereof, or with an anti-PD-L1 antibody or antigen-binding fragment thereof.
E23. The method of any one of E1-E22, wherein said subject is refractory to treatment with an anti-PD-1 antibody or antigen-binding fragment thereof.
E24. The method of any one of E1-E23, wherein said subject is refractory to treatment with an anti-LILRB1 antibody or antigen-binding fragment thereof, an anti-LILRB2 antibody or antigen-binding fragment thereof, or an anti-HLA-G antibody or antigen-binding fragment thereof.
E25. The method of any one of E1-E24, wherein said subject is refractory to treatment with an anti-LILRB1 antibody or antigen-binding fragment thereof.
E26. A method for increasing CD8+ T-cell mediated cytotoxicity in a subject that has cancer, comprising administering to the subject a therapeutically effective amount of (i) a first antibody, or antigen-binding fragment thereof, that binds PD-1, PD-L1, or PD-L2; and (ii) a second antibody, or antigen-binding fragment thereof, that binds LILRB1, LILRB2, or HLA-G.
E27 The method of E26, wherein said first antibody, or antigen-binding fragment thereof, binds PD-1.
E28. The method of E26 or E27, wherein said first antibody, or antigen-binding fragment thereof, binds human PD-1.
E29. The method of any one of E26-E28, wherein said first antibody is nivolumab, pembrolizumab, pidilizumab or an antigen binding fragment thereof.
E30. The method of E26, wherein said first antibody, or antigen-binding fragment thereof, binds PD-L1, PD-L2, or both.
E30. The method of E26 or E30, wherein said first antibody, or antigen-binding fragment thereof, binds human PD-L1, PD-L2, or both.
E32. The method of any one of E26 and E30-E31, wherein said first antibody is avelumab, atezolizumab, durvalumab or an antigen binding fragment thereof.
E33. The method of any one of E26-E32, wherein said second antibody, or antigen-binding fragment thereof, binds LILRB1, LILRB2, or both.
E34. The method of any one of E26-E33, wherein said second antibody, or antigen-binding fragment thereof, binds human LILRB1, human LILRB2, or both.
E35. The method of any one of E26-E34, wherein said second antibody, or antigen-binding fragment thereof, binds both human LILRB1 and human LILRB2.
E36. The method of any one of E26-E32, wherein said second antibody, or antigen-binding fragment thereof, binds HLA-G.
E37. The method of any one of E26-E32 and E36, wherein said second antibody, or antigen-binding fragment thereof, binds human HLA-G.
E38. The method of any one of E26-E37, wherein said first antibody, or antigen-binding fragment thereof, and said second antibody, or antigen-binding fragment thereof, are administered concurrently.
E39. The method of any one of E26-E37, wherein said first antibody, or antigen-binding fragment thereof, and said second antibody, or antigen-binding fragment thereof, are administered sequentially.
E40. The method of E39, wherein one or more doses of said first antibody, or antigen-binding fragment thereof, are administered prior to administering said second antibody, or antigen-binding fragment thereof.
E41. The method of E39, wherein one or more doses of said second antibody, or antigen-binding fragment thereof, are administered prior to administering said first antibody, or antigen-binding fragment thereof.
E42. A method for increasing CD8+ T-cell mediated cytotoxicity in a subject that has cancer, comprising administering to the subject a therapeutically effective amount of (i) an antibody, or antigen-binding fragment thereof, that binds PD-1, PD-L1, or PD-L2; and (ii) a short interfering RNA (siRNA) that reduces the expression level of HLA-G, as compared to a control.
E43. The method of E42, wherein said siRNA comprises the nucleotide sequence of SEQ ID NO: 31.
E44. The method of any one of E26-E43, wherein said subject is a human.
E45. The method of any one of E26-E44, wherein said cancer is a solid tumor.
E46. The method of any one of E26-E45, wherein the cancer is brain cancer, bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), Squamous Cell Carcinoma of the Head and Neck (SCCHN), chronic myelogenous leukemia (CML), small lymphocytic lymphoma (SLL), malignant mesothelioma, colorectal cancer, or gastric cancer.
E47. The method of any one of E26-E46, wherein said CD8+ T-cell mediated cytotoxicity is assessed by IFN-γ expression level in said subject.
E48. The method of any one of E26-E47, wherein said subject is refractory to treatment with an anti-PD-1 antibody or antigen-binding fragment thereof, or with an anti-PD-L1 antibody or antigen-binding fragment thereof.
E49. The method of any one of E26-E48, wherein said subject is refractory to treatment with an anti-PD-1 antibody or antigen-binding fragment thereof.
E50. The method of any one of E26-E49, wherein said subject is refractory to treatment with an anti-LILRB1 antibody or antigen-binding fragment thereof, an anti-LILRB2 antibody or antigen-binding fragment thereof, or an anti-HLA-G antibody or antigen-binding fragment thereof.
E51. The method of any one of E26-E50, wherein said subject is refractory to treatment with an anti-LILRB1 antibody or antigen-binding fragment thereof.
E52. A method for treating a subject that has a tumor, wherein said subject is refractory to treatment with an anti-PD-1 antibody or antigen-binding fragment thereof, or with an anti-PD-L1 antibody or antigen-binding fragment thereof, comprising: administering to the subject a therapeutically effective amount of (i) a first antibody, or antigen-binding fragment thereof, that binds PD-1, PD-L1, or PD-L2; and (ii) a second antibody, or antigen-binding fragment thereof, that binds LILRB1, LILRB2, or HLA-G.
E53. The method of E52, wherein said subject is also refractory to treatment with an anti-LILRB1 antibody or antigen-binding fragment thereof, an anti-LILRB2 antibody or antigen-binding fragment thereof, or an anti-HLA-G antibody or antigen-binding fragment thereof.
E54. The method of E52 or E53, wherein said first antibody, or antigen-binding fragment thereof, binds PD-1.
E55. The method of any one of E52-E54, wherein said first antibody, or antigen-binding fragment thereof, binds human PD-1.
E56. The method of any one of E52-E55, wherein said first antibody is nivolumab, pembrolizumab, pidilizumab, or an antigen binding fragment thereof.
E57. The method of E52 or E53, wherein said first antibody, or antigen-binding fragment thereof, binds PD-L1, PD-L2, or both.
E58. The method of any one of E52-E53 and E57, wherein said first antibody, or antigen-binding fragment thereof, binds human PD-L1, PD-L2, or both.
E59. The method of any one of E52-E53 and E57-E58, wherein said first antibody is avelumab, atezolizumab, durvalumab, or an antigen binding fragment thereof.
E60. The method of any one of E52-E59, wherein said second antibody, or antigen-binding fragment thereof, binds LILRB1, LILRB2, or both.
E61. The method of any one of E52-E60, wherein said second antibody, or antigen-binding fragment thereof, binds human LILRB1, human LILRB2, or both.
E62. The method of any one of E52-E61, wherein said second antibody, or antigen-binding fragment thereof, binds both human LILRB1 and human LILRB2.
E63. The method of any one of E52-E59, wherein said second antibody, or antigen-binding fragment thereof, binds HLA-G.
E64. The method of any one of E52-E59 and E63, wherein said second antibody, or antigen-binding fragment thereof, binds human HLA-G.
E65. The method of any one of E52-E64, wherein said first antibody, or antigen-binding fragment thereof, and said second antibody, or antigen-binding fragment thereof, are administered concurrently.
E66. The method of any one of E52-E64, wherein said first antibody, or antigen-binding fragment thereof, and said second antibody, or antigen-binding fragment thereof, are administered sequentially.
E67. The method of E66, wherein one or more doses of said first antibody, or antigen-binding fragment thereof, are administered prior to administering said second antibody, or antigen-binding fragment thereof.
E58. The method of E66, wherein one or more doses of said second antibody, or antigen-binding fragment thereof, are administered prior to administering said first antibody, or antigen-binding fragment thereof.
E69. A method for treating a subject that has a tumor, wherein said subject is refractory to treatment with an anti-PD-1 antibody or antigen-binding fragment thereof, or an anti-PD-L1 antibody or antigen-binding fragment thereof, comprising: administering to the subject a therapeutically effective amount of (i) an antibody, or antigen-binding fragment thereof, that binds PD-1, PD-L1, or PD-L2; and (ii) a short interfering RNA (siRNA) that reduces the expression level of HLA-G, as compared to a control.
E70. The method of E69, wherein said subject is also refractory to treatment with an anti-LILRB1 antibody or antigen-binding fragment thereof, an anti-LILRB2 antibody or antigen-binding fragment thereof, an anti-HLA-G antibody or antigen-binding fragment thereof, or a short interfering RNA (siRNA) that reduces the expression level of HLA-G as compared to a control.
E71. The method of E70, wherein said siRNA comprises the nucleotide sequence of SEQ ID NO: 31.
E72. The method of any one of E52-E71, wherein said subject is a human.
E73. The method of any one of E52-E72, wherein the cancer is a solid tumor.
E74. The method of any one of E52-E73, wherein the cancer is brain cancer, bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), Squamous Cell Carcinoma of the Head and Neck (SCCHN), chronic myelogenous leukemia (CML), small lymphocytic lymphoma (SLL), malignant mesothelioma, colorectal cancer, or gastric cancer.
E75. The method of any one of E52-E74, wherein said subject is refractory to treatment with an anti-PD-1 antibody or antigen-binding fragment thereof.
E76. The method of any one of E52-E75, wherein said subject is refractory to treatment with an anti-LILRB1 antibody or antigen-binding fragment thereof.
E77. The method of any one of E1-E3, E8-E28, E33-E55, and E60-E76, wherein said first antibody, or antigen-binding fragment thereof, binds PD-1 and comprises:
   (i) a heavy chain variable region (VH) that comprises:
      (a) a VH complementarity determining region one (CDR-H1) comprising the amino acid sequence of SEQ ID NO: 19,
      (b) a VH CDR-H2 comprising the amino acid sequence of SEQ ID NO: 20; and
      (c) a VH CDR-H3 comprising the amino acid sequence of SEQ ID NO: 21; and
   (ii) a light chain variable region (VL) that comprises:
      (a) a VL complementarity determining region one (CDR-L1) comprising the amino acid sequence of SEQ ID NO: 22,
      (b) a VL CDR-L2 comprising the amino acid sequence of SEQ ID NO: 23; and
      (c) a VL CDR-L3 comprising the amino acid sequence of SEQ ID NO: 24.
E78. The method of any one of E1-E3, E8-E28, E33-E55, and E60-E77, wherein said first antibody, or antigen-binding fragment thereof, comprises a VH that comprises the amino acid sequence of SEQ ID NO: 25.
E79. The method of any one of E1-E3, E8-E28, E33-E55, and E60-E78, wherein said first antibody, or antigen-binding fragment thereof, comprises a heavy chain constant region (CH) that comprises the amino acid sequence of SEQ ID NO: 29.
E80. The method of any one of E1-E3, E8-E28, E33-E55, and E60-E79, wherein said first antibody, or antigen-binding fragment thereof, comprises a VL that comprises the amino acid sequence of SEQ ID NO: 26.
E81. The method of any one of E1-E3, E8-E28, E33-E55, and E60-E80, wherein said first antibody, or antigen-binding fragment thereof, comprises a light chain constant region (CL) that comprises the amino acid sequence of SEQ ID NO: 30.
E82. The method of any one of E1-E3, E8-E28, E33-E55, and E60-E81, wherein said first antibody, or antigen-binding fragment thereof, comprises a heavy chain that comprises the amino acid sequence of SEQ ID NO: 27.
E83. The method of any one of E1-E3, E8-E28, E33-E55, and E60-E82, wherein said first antibody, or antigen-binding fragment thereof, comprises a light chain that comprises the amino acid sequence of SEQ ID NO: 28.
E84a. The method of any one of E1-E10, E13-E16, E19-E35, E38-E41, E44-E62, E65-E69, and E72-E83, wherein said second antibody, or antigen-binding fragment thereof, binds LILRB1 and comprises:
   (i) a heavy chain variable region (VH) that comprises:
      (a) a VH complementarity determining region one (CDR-H1) comprising the amino acid sequence of SEQ ID NO: 36,
      (b) a VH CDR-H2 comprising the amino acid sequence of SEQ ID NO: 37; and
      (c) a VH CDR-H3 comprising the amino acid sequence of SEQ ID NO: 38; and
   (ii) a light chain variable region (VL) that comprises:
      (a) a VL complementarity determining region one (CDR-L1) comprising the amino acid sequence of SEQ ID NO: 39,
      (b) a VL CDR-L2 comprising the amino acid sequence of SEQ ID NO: 40; and
      (c) a VL CDR-L3 comprising the amino acid sequence of SEQ ID NO: 41.
E84b. The method of any one of E1-E10, E13-E16, E19-E35, E38-E41, E44-E62, E65-E69, E72-E83, and E84a, wherein said second antibody, or antigen-binding fragment thereof, comprises a VH that comprises the amino acid sequence of SEQ ID NO: 42.
E84c. The method of any one of E1-E10, E13-E16, E19-E35, E38-E41, E44-E62, E65-E69, E72-E83, and E84a-E84b, wherein said second antibody, or antigen-binding fragment thereof, comprises a heavy chain constant region (CH) that comprises the amino acid sequence of SEQ ID NO: 46.
E84d. The method of any one of E1-E10, E13-E16, E19-E35, E38-E41, E44-E62, E65-E69, E72-E83, and E84a-E84c, wherein said second antibody, or antigen-binding fragment thereof, comprises a VL that comprises the amino acid sequence of SEQ ID NO: 43.
E84e. The method of any one of E1-E10, E13-E16, E19-E35, E38-E41, E44-E62, E65-E69, E72-E83, and E84a-E84d, wherein said second antibody, or antigen-binding fragment thereof, comprises a light chain constant region (CL) that comprises the amino acid sequence of SEQ ID NO: 47.
E84f. The method of any one of E1-E10, E13-E16, E19-E35, E38-E41, E44-E62, E65-E69, E72-E83, and E84a-E84e, wherein said second antibody, or antigen-binding fragment thereof, comprises a heavy chain that comprises the amino acid sequence of SEQ ID NO: 44.
E84g. The method of any one of E1-E10, E13-E16, E19-E35, E38-E41, E44-E62, E65-E69, E72-E83, and E84a-E84f, wherein said second antibody, or antigen-binding fragment thereof, comprises a light chain that comprises the amino acid sequence of SEQ ID NO: 45.
E84h. The method of any one of E1-E10, E13-E16, E19-E35, E38-E41, E44-E62, E65-E69, and E72-E83, wherein said second antibody, or antigen-binding fragment thereof, binds LILRB1, and competes for binding to LILRB1 with an antibody or antigen-binding fragment thereof of any one of E84a-E84g.
E85a. The method of any one of E1-E10, E13-E16, E19-E35, E38-E41, E44-E62, E65-E69, and E72-E83, wherein said second antibody, or antigen-binding fragment thereof, binds LILRB1 and comprises:
   (i) a heavy chain variable region (VH) that comprises:
      (a) a VH complementarity determining region one (CDR-H1) comprising the amino acid sequence of SEQ ID NO: 48,
      (b) a VH CDR-H2 comprising the amino acid sequence of SEQ ID NO: 49; and
      (c) a VH CDR-H3 comprising the amino acid sequence of SEQ ID NO: 50; and
   (ii) a light chain variable region (VL) that comprises:
      (a) a VL complementarity determining region one (CDR-L1) comprising the amino acid sequence of SEQ ID NO: 51,
      (b) a VL CDR-L2 comprising the amino acid sequence of SEQ ID NO: 52; and
      (c) a VL CDR-L3 comprising the amino acid sequence of SEQ ID NO: 53.
E85b. The method of any one of E1-E10, E13-E16, E19-E35, E38-E41, E44-E62, E65-E69, E72-E83, and E85a, wherein said second antibody, or antigen-binding fragment thereof, comprises a VH that comprises the amino acid sequence of SEQ ID NO: 54.
E85c. The method of any one of E1-E10, E13-E16, E19-E35, E38-E41, E44-E62, E65-E69, E72-E83, and E85a-E85b, wherein said second antibody, or antigen-binding fragment thereof, comprises a heavy chain constant region (CH) that comprises the amino acid sequence of SEQ ID NO: 46.
E85d. The method of any one of E1-E10, E13-E16, E19-E35, E38-E41, E44-E62, E65-E69, E72-E83, and E85a-E85c, wherein said second antibody, or antigen-binding fragment thereof, comprises a VL that comprises the amino acid sequence of SEQ ID NO: 55.
E85e. The method of any one of E1-E10, E13-E16, E19-E35, E38-E41, E44-E62, E65-E69, E72-E83, and E85a-E85d, wherein said second antibody, or antigen-binding fragment thereof, comprises a light chain constant region (CL) that comprises the amino acid sequence of SEQ ID NO: 30.
E85f. The method of any one of E1-E10, E13-E16, E19-E35, E38-E41, E44-E62, E65-E69, E72-E83, and E85a-E85e, wherein said second antibody, or antigen-binding fragment thereof, comprises a heavy chain that comprises the amino acid sequence of SEQ ID NO: 56.
E85g. The method of any one of E1-E10, E13-E16, E19-E35, E38-E41, E44-E62, E65-E69, E72-E83, and E85a-E85f, wherein said second antibody, or antigen-binding fragment thereof, comprises a light chain that comprises the amino acid sequence of SEQ ID NO: 57.
E85h. The method of any one of E1-E10, E13-E16, E19-E35, E38-E41, E44-E62, E65-E69, and E72-E83, wherein said second antibody, or antigen-binding fragment thereof, binds LILRB1, and competes for binding to LILRB1 with an antibody or antigen-binding fragment thereof of any one of E85a-E85g.
E86a. The method of any one of E1-E10, E13-E16, E19-E35, E38-E41, E44-E62, E65-E69, and E72-E83, wherein said second antibody, or antigen-binding fragment thereof, binds LILRB1 and comprises:
   (i) a heavy chain variable region (VH) that comprises:
      (a) a VH complementarity determining region one (CDR-H1) comprising the amino acid sequence of SEQ ID NO: 58,
      (b) a VH CDR-H2 comprising the amino acid sequence of SEQ ID NO: 59; and
      (c) a VH CDR-H3 comprising the amino acid sequence of SEQ ID NO: 60; and
   (ii) a light chain variable region (VL) that comprises:
      (a) a VL complementarity determining region one (CDR-L1) comprising the amino acid sequence of SEQ ID NO: 61,
      (b) a VL CDR-L2 comprising the amino acid sequence of SEQ ID NO: 62; and
      (c) a VL CDR-L3 comprising the amino acid sequence of SEQ ID NO: 63.
E86b. The method of any one of E1-E10, E13-E16, E19-E35, E38-E41, E44-E62, E65-E69, E72-E83, and E86a, wherein said second antibody, or antigen-binding fragment thereof, comprises a VH that comprises the amino acid sequence of SEQ ID NO: 64.
E86c. The method of any one of E1-E10, E13-E16, E19-E35, E38-E41, E44-E62, E65-E69, E72-E83, and E86a-E86b, wherein said second antibody, or antigen-binding fragment thereof, comprises a heavy chain constant region (CH) that comprises the amino acid sequence of SEQ ID NO: 46.
E86d. The method of any one of E1-E10, E13-E16, E19-E35, E38-E41, E44-E62, E65-E69, E72-E83, and E86a-E86c, wherein said second antibody, or antigen-binding fragment thereof, comprises a VL that comprises the amino acid sequence of SEQ ID NO: 65.
E86e. The method of any one of E1-E10, E13-E16, E19-E35, E38-E41, E44-E62, E65-E69, E72-E83, and E86a-E86d, wherein said second antibody, or antigen-binding fragment thereof, comprises a light chain constant region (CL) that comprises the amino acid sequence of SEQ ID NO: 47.
E86f. The method of any one of E1-E10, E13-E16, E19-E35, E38-E41, E44-E62, E65-E69, E72-E83, and E86a-E86e, wherein said second antibody, or antigen-binding fragment thereof, comprises a heavy chain that comprises the amino acid sequence of SEQ ID NO: 66.
E86g. The method of any one of E1-E10, E13-E16, E19-E35, E38-E41, E44-E62, E65-E69, E72-E83, and E86a-E86f, wherein said second antibody, or antigen-binding fragment thereof, comprises a light chain that comprises the amino acid sequence of SEQ ID NO: 67.
E86h. The method of any one of E1-E10, E13-E16, E19-E35, E38-E41, E44-E62, E65-E69, and E72-E83, wherein said second antibody, or antigen-binding fragment thereof, binds LILRB1, and competes for binding to LILRB1 with an antibody or antigen-binding fragment thereof of any one of E86a-E86g.
E87a. An antibody, or antigen-binding fragment thereof, that binds LILRB1 and comprises:
   (i) a heavy chain variable region (VH) that comprises:
      (a) a VH complementarity determining region one (CDR-H1) comprising the amino acid sequence of SEQ ID NO: 36,
      (b) a VH CDR-H2 comprising the amino acid sequence of SEQ ID NO: 37; and
      (c) a VH CDR-H3 comprising the amino acid sequence of SEQ ID NO: 38; and
   (ii) a light chain variable region (VL) that comprises:
      (a) a VL complementarity determining region one (CDR-L1) comprising the amino acid sequence of SEQ ID NO: 39,
      (b) a VL CDR-L2 comprising the amino acid sequence of SEQ ID NO: 40; and
      (c) a VL CDR-L3 comprising the amino acid sequence of SEQ ID NO: 41.
E87b. The antibody, or antigen-binding fragment thereof, of E87a, comprising a VH that comprises the amino acid sequence of SEQ ID NO: 42.
E87c. The antibody, or antigen-binding fragment thereof, of E87a or E87b, comprising a heavy chain constant region (CH) that comprises the amino acid sequence of SEQ ID NO: 46.
E87d. The antibody, or antigen-binding fragment thereof, of any one of E87a-E87c, comprising a VL that comprises the amino acid sequence of SEQ ID NO: 43.
E87e. The antibody, or antigen-binding fragment thereof, of any one of E87a-E87d comprising a light chain constant region (CL) that comprises the amino acid sequence of SEQ ID NO: 47.
E87f. The antibody, or antigen-binding fragment thereof, of any one of E87a-E87e, comprising a heavy chain that comprises the amino acid sequence of SEQ ID NO: 44.
E87g. The antibody, or antigen-binding fragment thereof, of any one of E87a-E87f, comprising a light chain that comprises the amino acid sequence of SEQ ID NO: 45.
E87h. An antibody, or antigen-binding fragment thereof, that binds LILRB1, and competes for binding to LILRB1 with an antibody or antigen-binding fragment thereof of any one of E87a-E87g.
E87i. An antibody, or antigen-binding fragment thereof, that binds the D4 domain of LILRB1, wherein said D4 domain comprises residues 313-409 of SEQ ID NO: 3.
E87j. The antibody, or antigen-binding fragment thereof, of E87a-87i, wherein the antibody, or antigen binding fragment thereof, binds LILRB1 with a K_{D} value of or less than: about 10 nM, about 5 nM, about 2 nM, about 1 nM, about 900 pM, about 800 pM, about 700 pM, about 600 pM, about 500 pM, about 400 pM, about 300 pM, about 250 pM, about 200 pM, about 150 pM, about 100 pM, about 50 pM, about 40 pM, about 30 pM, about 25 pM, about 20 pM, about 15 pM, about 10 pM, about 5 pM, or about 1 pM.
E87k. The antibody, or antigen-binding fragment thereof, of E87j, wherein said K_{D} value is measured by surface plasmon resonance (SPR), optionally using a Biacore® T200 instrument.
E871. The antibody, or antigen-binding fragment thereof, of E87j, wherein said Ko value is measured by bio-layer interferometry (BLI), optionally using a ForteBio Octet instrument.
E88a. An antibody, or antigen-binding fragment thereof, that binds LILRB1 and comprises:
   (i) a heavy chain variable region (VH) that comprises:
      (a) a VH complementarity determining region one (CDR-H1) comprising the amino acid sequence of SEQ ID NO: 48,
      (b) a VH CDR-H2 comprising the amino acid sequence of SEQ ID NO: 49; and
      (c) a VH CDR-H3 comprising the amino acid sequence of SEQ ID NO: 50; and
   (ii) a light chain variable region (VL) that comprises:
      (a) a VL complementarity determining region one (CDR-L1) comprising the amino acid sequence of SEQ ID NO: 51,
      (b) a VL CDR-L2 comprising the amino acid sequence of SEQ ID NO: 52; and
      (c) a VL CDR-L3 comprising the amino acid sequence of SEQ ID NO: 53.
E88b. The antibody, or antigen-binding fragment thereof, of E88a, comprising a VH that comprises the amino acid sequence of SEQ ID NO: 54.
E88c. The antibody, or antigen-binding fragment thereof, of E88a or E88b, comprising a heavy chain constant region (CH) that comprises the amino acid sequence of SEQ ID NO: 46.
E88d. The antibody, or antigen-binding fragment thereof, of any one of E88a-E88c, comprising a VL that comprises the amino acid sequence of SEQ ID NO: 55.
E88e. The antibody, or antigen-binding fragment thereof, of any one of E88a-E88d comprising a light chain constant region (CL) that comprises the amino acid sequence of SEQ ID NO: 30.
E88f. The antibody, or antigen-binding fragment thereof, of any one of E88a-E88e, comprising a heavy chain that comprises the amino acid sequence of SEQ ID NO: 56.
E88g. The antibody, or antigen-binding fragment thereof, of any one of E88a-E88f, comprising a light chain that comprises the amino acid sequence of SEQ ID NO: 57.
E88h. An antibody, or antigen-binding fragment thereof, that binds LILRB1, and competes for binding to LILRB1 with an antibody or antigen-binding fragment thereof of any one of E88a-E88g.
E88i. An antibody, or antigen-binding fragment thereof, that binds the D3 domain of LILRB1, wherein said D3 domain comprises residues 222-312 of SEQ ID NO: 3.
E88j. The antibody, or antigen-binding fragment thereof, of E88a-88i, wherein the antibody, or antigen binding fragment thereof, binds LILRB1 with a Ko value of or less than: about 10 nM, about 5 nM, about 2 nM, about 1 nM, about 900 pM, about 800 pM, about 700 pM, about 600 pM, about 500 pM, about 400 pM, about 300 pM, about 250 pM, about 200 pM, about 150 pM, about 100 pM, about 50 pM, about 40 pM, about 30 pM, about 25 pM, about 20 pM, about 15 pM, about 10 pM, about 5 pM, or about 1 pM.
E88k. The antibody, or antigen-binding fragment thereof, of E88j, wherein said Ko value is measured by surface plasmon resonance (SPR), optionally using a Biacore T200 instrument.
E881. The antibody, or antigen-binding fragment thereof, of E88j, wherein said K_{D} value is measured by bio-layer interferometry (BLI), optionally using a ForteBio Octet instrument.
E89a. An antibody, or antigen-binding fragment thereof, that binds LILRB1 and comprises:
   (i) a heavy chain variable region (VH) that comprises:
      (a) a VH complementarity determining region one (CDR-H1) comprising the amino acid sequence of SEQ ID NO: 58,
      (b) a VH CDR-H2 comprising the amino acid sequence of SEQ ID NO: 59; and
      (c) a VH CDR-H3 comprising the amino acid sequence of SEQ ID NO: 60; and
   (ii) a light chain variable region (VL) that comprises:
      (a) a VL complementarity determining region one (CDR-L1) comprising the amino acid sequence of SEQ ID NO: 61,
      (b) a VL CDR-L2 comprising the amino acid sequence of SEQ ID NO: 62; and
      (c) a VL CDR-L3 comprising the amino acid sequence of SEQ ID NO: 63.
E89b. The antibody, or antigen-binding fragment thereof, of E89a, comprising a VH that comprises the amino acid sequence of SEQ ID NO: 64.
E89c. The antibody, or antigen-binding fragment thereof, of E89a or E89b, comprising a heavy chain constant region (CH) that comprises the amino acid sequence of SEQ ID NO: 46.
E89d. The antibody, or antigen-binding fragment thereof, of any one of E89a-E89c, comprising a VL that comprises the amino acid sequence of SEQ ID NO: 65.
E89e. The antibody, or antigen-binding fragment thereof, of any one of E89a-E89d comprising a light chain constant region (CL) that comprises the amino acid sequence of SEQ ID NO: 47.
E89f. The antibody, or antigen-binding fragment thereof, of any one of E89a-E89e, comprising a heavy chain that comprises the amino acid sequence of SEQ ID NO: 66.
E89g. The antibody, or antigen-binding fragment thereof, of any one of E89a-E89f, comprising a light chain that comprises the amino acid sequence of SEQ ID NO: 67.
E89h. An antibody, or antigen-binding fragment thereof, that binds LILRB1, and competes for binding to LILRB1 with an antibody or antigen-binding fragment thereof of any one of E89a-E89g.
E89i. An antibody, or antigen-binding fragment thereof, that binds the D4 domain of LILRB1, wherein said D4 domain comprises residues 313-409 of SEQ ID NO: 3.
E89j. The antibody, or antigen-binding fragment thereof, of E89a-89i, wherein the antibody, or antigen binding fragment thereof, binds LILRB1 with a K_{D} value of or less than: about 10 nM, about 5 nM, about 2 nM, about 1 nM, about 900 pM, about 800 pM, about 700 pM, about 600 pM, about 500 pM, about 400 pM, about 300 pM, about 250 pM, about 200 pM, about 150 pM, about 100 pM, about 50 pM, about 40 pM, about 30 pM, about 25 pM, about 20 pM, about 15 pM, about 10 pM, about 5 pM, or about 1 pM.
E89k. The antibody, or antigen-binding fragment thereof, of E89j, wherein said Ko value is measured by surface plasmon resonance (SPR), optionally using a Biacore T200 instrument.
E891. The antibody, or antigen-binding fragment thereof, of E89j, wherein said K_{D} value is measured by bio-layer interferometry (BLI), optionally using a ForteBio Octet instrument.
E90. A composition for use in increasing IFN-γ expression level in a subject that has cancer, comprising (i) a first antibody, or antigen-binding fragment thereof, that binds PD-1, PD-L1, or PD-L2; and (ii) a second antibody, or antigen-binding fragment thereof, that binds LILRB1, LILRB2, or HLA-G.
E91. Use of a composition for increasing IFN-γ expression level in a subject that has cancer, said composition comprises (i) a first antibody, or antigen-binding fragment thereof, that binds PD-1, PD-L1, or PD-L2; and (ii) a second antibody, or antigen-binding fragment thereof, that binds LILRB1, LILRB2, or HLA-G.
E92. Use of a composition in the manufacture of a medicament for increasing IFN-γ expression level in a subject that has cancer, said composition comprises (i) a first antibody, or antigen-binding fragment thereof, that binds PD-1, PD-L1, or PD-L2; and (ii) a second antibody, or antigen-binding fragment thereof, that binds LILRB1, LILRB2, or HLA-G.
E93. A composition for use in increasing IFN-γ expression level in a subject that has cancer, comprising (i) an antibody, or antigen-binding fragment thereof, that binds PD-1, PD-L1, or PD-L2; and (ii) a short interfering RNA (siRNA) that reduces the expression level of HLA-G, as compared to a control.
E94. Use of a composition for increasing IFN-γ expression level in a subject that has cancer, said composition comprises (i) an antibody, or antigen-binding fragment thereof, that binds PD-1, PD-L1, or PD-L2; and (ii) a short interfering RNA (siRNA) that reduces the expression level of HLA-G, as compared to a control.
E95. Use of a composition in the manufacture of a medicament for increasing IFN-γ expression level in a subject that has cancer, said composition comprises (i) an antibody, or antigen-binding fragment thereof, that binds PD-1, PD-L1, or PD-L2; and (ii) a short interfering RNA (siRNA) that reduces the expression level of HLA-G, as compared to a control.
E96. A composition for use in increasing CD8+ T-cell mediated cytotoxicity in a subject that has cancer, comprising (i) a first antibody, or antigen-binding fragment thereof, that binds PD-1, PD-L1, or PD-L2; and (ii) a second antibody, or antigen-binding fragment thereof, that binds LILRB1, LILRB2, or HLA-G.
E97. Use of a composition for increasing CD8+ T-cell mediated cytotoxicity in a subject that has cancer, said composition comprises (i) a first antibody, or antigen-binding fragment thereof, that binds PD-1, PD-L1, or PD-L2; and (ii) a second antibody, or antigen-binding fragment thereof, that binds LILRB1, LILRB2, or HLA-G.
E98. Use of a composition in the manufacture of a medicament for increasing CD8+ T-cell mediated cytotoxicity in a subject that has cancer, said composition comprises (i) a first antibody, or antigen-binding fragment thereof, that binds PD-1, PD-L1, or PD-L2; and (ii) a second antibody, or antigen-binding fragment thereof, that binds LILRB1, LILRB2, or HLA-G.
E99. A composition for use in increasing CD8+ T-cell mediated cytotoxicity in a subject that has cancer, comprising (i) an antibody, or antigen-binding fragment thereof, that binds PD-1, PD-L1, or PD-L2; and (ii) a short interfering RNA (siRNA) that reduces the expression level of HLA-G, as compared to a control.
E100. Use of a composition for increasing CD8+ T-cell mediated cytotoxicity in a subject that has cancer, said composition comprises (i) an antibody, or antigen-binding fragment thereof, that binds PD-1, PD-L1, or PD-L2; and (ii) a short interfering RNA (siRNA) that reduces the expression level of HLA-G, as compared to a control.
E101. Use of a composition in the manufacture of a medicament for increasing CD8+ T-cell mediated cytotoxicity in a subject that has cancer, said composition comprises (i) an antibody, or antigen-binding fragment thereof, that binds PD-1, PD-L1, or PD-L2; and (ii) a short interfering RNA (siRNA) that reduces the expression level of HLA-G, as compared to a control.
E102. A composition for use in treating cancer in a subject, wherein said subject is refractory to treatment with an anti-PD-1 antibody or antigen-binding fragment thereof, or with an anti-PD-L1 antibody or antigen-binding fragment thereof, and wherein said composition comprises (i) a first antibody, or antigen-binding fragment thereof, that binds PD-1, PD-L1, or PD-L2; and (ii) a second antibody, or antigen-binding fragment thereof, that binds LILRB1, LILRB2, or HLA-G.
E103. Use of a composition for treating cancer in a subject, wherein said subject is refractory to treatment with an anti-PD-1 antibody or antigen-binding fragment thereof, or with an anti-PD-L1 antibody or antigen-binding fragment thereof, and wherein said composition comprises (i) a first antibody, or antigen-binding fragment thereof, that binds PD-1, PD-L1, or PD-L2; and (ii) a second antibody, or antigen-binding fragment thereof, that binds LILRB1, LILRB2, or HLA-G.
E104. Use of a composition in the manufacture of a medicament for treating cancer in a subject, wherein said subject is refractory to treatment with an anti-PD-1 antibody or antigen-binding fragment thereof, or with an anti-PD-L1 antibody or antigen-binding fragment thereof, and wherein said composition comprises (i) a first antibody, or antigen-binding fragment thereof, that binds PD-1, PD-L1, or PD-L2; and (ii) a second antibody, or antigen-binding fragment thereof, that binds LILRB1, LILRB2, or HLA-G.
E105. A composition for use in treating cancer in a subject, wherein said subject is refractory to treatment with an anti-PD-1 antibody or antigen-binding fragment thereof, or with an anti-PD-L1 antibody or antigen-binding fragment thereof, and wherein said composition comprises (i) an antibody, or antigen-binding fragment thereof, that binds PD-1, PD-L1, or PD-L2; and (ii) a short interfering RNA (siRNA) that reduces the expression level of HLA-G, as compared to a control.
E106. Use of a composition for treating cancer in a subject, wherein said subject is refractory to treatment with an anti-PD-1 antibody or antigen-binding fragment thereof, or with an anti-PD-L1 antibody or antigen-binding fragment thereof, and wherein said composition comprises (i) an antibody, or antigen-binding fragment thereof, that binds PD-1, PD-L1, or PD-L2; and (ii) a short interfering RNA (siRNA) that reduces the expression level of HLA-G, as compared to a control.
E107. Use of a composition in the manufacture of a medicament for treating cancer in a subject, wherein said subject is refractory to treatment with an anti-PD-1 antibody or antigen-binding fragment thereof, or with an anti-PD-L1 antibody or antigen-binding fragment thereof, and wherein said composition comprises (i) an antibody, or antigen-binding fragment thereof, that binds PD-1, PD-L1, or PD-L2; and (ii) a short interfering RNA (siRNA) that reduces the expression level of HLA-G, as compared to a control.
E108. A method of enhancing the T-cell mediated cytotoxicity against a cancer cell in a subject, comprising administering to the subject a therapeutically effective amount of (i) a Bi-specific T-cell engager (BiTER) molecule; and (ii) an antibody, or antigen-binding fragment thereof, that binds LILRB1, LILRB2, or HLA-G.
E109. The method of E108, wherein said antibody, or antigen-binding fragment thereof, binds LILRB1.
E110. A method for treating a subject that has a tumor, wherein said subject is refractory to treatment with an anti-PD-1 antibody or antigen-binding fragment thereof, or with an anti-PD-L1 antibody or antigen-binding fragment thereof, comprising: administering to the subject a therapeutically effective amount of an antibody, or antigen-binding fragment thereof, that binds LILRB1, LILRB2, or HLA-G.
E111. The method of E110, wherein said antibody, or antigen-binding fragment thereof, binds LILRB1.

### BRIEF DESCRIPTION OF THE FIGURES

FIGS. 1A-1D are graphs showing FACS analysis or RNA expression analysis of human CD8+ T cells from healthy donor PBMCs. FIGS. 1A-1B are representative FACS plots showing CD8+ T cell subsets, as defined by CD45RA and CCR7 expression, and their corresponding LILRB1 expression levels. Results from multiple donors were quantitated in FIG. 1C, which summarizes the quantitation of LILRB1 expression in Tem and Temra CD8+ T cells, as determined by FACS analysis (n=8). **P<0.01 with paired students' t-test. FIG. 1D shows LILRB1 mRNA expression levels in Tn, Tem and Temra subsets from both CD4+ and CD8+ T cells, as determined by RNA-sequencing analysis (n=2-6).

FIGS. 2A-2D show that LILRB1 inhibits CD8+ T cell activation and cytolytic effector function. FIG. 2A is a representative FACS plot showing HLA-G expression on SK-MEL-2 cells (SK2, dotted line) and HLA-G transfected SK2 cells (SK2.HLA-G, solid line). Gray-filled histogram is isotype control. FIG. 2B shows representative histograms for CD69 expression on Temra cells co-cultured with SK2 cells (line histograms) or SK2.HLA-G cells (gray-filled histograms) for 45 h in the presence of 0.4 nM BiTE® molecule (right panel) or without BiTER molecule (left panel). FIG. 2C shows the percentage CD8+ Temra cells that upregulate CD69 following co-culture with the indicated tumor cells for 18 h (left panel) or 45 h (right panel) in the presence of 0.4 nM BiTER molecule. Percentage of CD69+ cells (mean±SD) were determined by FACS analysis. Data is representative of three independent experiments with two individual donors as T cell source. *p<0.05 with students' t-test. FIG. 2D shows BiTER molecule-mediated cytotoxicity of isolated CD8+ Temra cells to indicated target cells in the presence of anti-LILRB1 blocking antibody (filled bars) or isotype control antibody (open bars). T cell and tumor cell co-culture with no BiTE® molecule was used as baseline to calculate specific lysis. Results shown as mean±SEM of T cells isolated from three donors (n=3).*p<0.05 with students' t-test.

FIG. 3 shows representative FACS plots of LILRB1 and PD-1 expression on CD8+ T cells isolated from the blood of healthy individuals after 48 hours of treatment with the indicated conditions. Histogram on the left shows isotype control staining of unstimulated CD8+ T cells. Results are representative of 5 donors analyzed across 3 experiments.

FIGS. 4A-4D show that LILRB1 and PD-1 are expressed by distinct CD8+ T cell subsets in tumor. FIG. 4A shows representative FACS plots of LILRB1 and PD-1 expression patterns on tumor infiltrated CD8+ T cells across three individual NSCLC patient tumor samples. Numbers represent percentage of cells in a given quadrant. FIG. 4B summarizes the quantitation of FIG. 4A from twelve NSCLC tumor patients. Percentage of total tumor CD8+ T cells that expressing LILRB1 (black filled), PD-1 (gray filled), or both (open) in each tumor is plotted. FIG. 4C shows the expression of CD69 from isolated tumor CD8+ T cells that were added to SK2 or SK2.HLA-G cell cultures together with BiTER molecule for 45 h. Expression of CD69 measured as mean florescence intensity (MFI) on CD8+ T cells was determined by FACS analysis. Each type of symbols represented data obtained from a given individual. Data from each individual and mean±SEM shown in plot. **p<0.01 with paired students' t-test. FIG. 4D shows BiTE® molecule-mediated cytotoxicity of isolated tumor CD8+ cells to indicated target cells in the presence of anti-LILRB1 blocking antibody (gray-filled bars) or isotype control antibody (open bars). Individual symbols represent data obtained from a given individual. Data from each individual and mean±SEM are shown. *p<0.05 with paired students' t-test. ns, not significant.

FIGS. 5A-5B show the pattern of co-expression of various immune checkpoint proteins. FIG. 5A summarizes data based on published hepatocellular carcinoma (HCC) tumor infiltrating CD8+ T cells; numbers refer to % of LILRB1+CD8+ cells (378 cells) or % of PD1+CD8+ cells (709 cells) that co-express indicated markers. FIG. 5B summarizes data from NSCLC tumor infiltrating CD8+ T cells; numbers refer to % of LILRB1+CD8+ cells (22 cells) or % of PD1+CD8+ cells (30 cells) that co-express indicated markers.

FIGS. 6A-6D show that certain sub-population of patients do not respond to anti-PD-1 treatment. However, some of the PD-1 non-responders show response to anti-LILRB1 blockade. FIG. 6A shows the result of an MLR assay with isolated human CD8+ T cells and allogenic MDDCs in the presence of the indicated blocking antibodies. IFNγ in day 5 supernatants were measured by ELISA. Each group of bars represents results from T cells derived from an individual donor. FIG. 6B-6D shows the result of an MLR assay with isolated total human T cells and allogenic MDDCs in the presence of indicated blocking antibodies. IFNγ in day 5 supernatants were measured by ELISA. Each group of bars represents results from T cells derived from an individual donor. FIG. 6B shows MLR results from donors that responded to anti-PD-1 treatment. FIG. 6C shows MLR results from anti-PD-1 non-responding donors that responded to anti-LILRB1 treatment. FIG. 6D shows MLR result of a given donor pair that responded to both anti-PD-1 and anti-LILRB1 treatments.

FIGS. 7A-7B show MLR assays with isolated total human T cells and allogenic DCs in the presence of the indicated blocking antibodies. IFNγ in day 5 supernatants were measured by ELISA. Data plotted as mean±SD*p<0.05 and **p<0.01 with students' t-test. Results are representative of three independent experiments with T cells from two individual donors.

FIGS. 8A-8D show that LILRB1 expression on the surface of CD8+ T cells is upregulated by effector cytokines and anti-PD1 blockade. FIG. 8A shows representative FACS plots of LILRB1 (left panel) and PD1 (right panel) expression on the surface of purified human CD8+ T cells upon TCR stimulation. Gray-filled histograms are control CD8+ T cells without stimulation. FIG. 8B shows quantitation from FIG. 8A (n=5 individual donors). *p<0.05 with students' t-test. ns, not significant. FIG. 8C shows representative FACS plots from purified human CD8+ Tem cells were activated with anti-CD3 plus anti-CD28 in the presence of 10 μg/ml recombinant human PDL1 (rhuPDL1, dashed lines) or human IgG1 (control, solid line). After 48 h, LILRB1 (left panel) and PD1 (right panel) expression was determined by FACS analysis. Gray-filled histograms are isotype staining controls. FIG. 8D shows quantitation of FIG. 8C. Representative data shown as mean±SD*p<0.05 with students' t-test. Three independent experiments were conducted with Tem cells isolated from three individual donors (n=3).

FIGS. 9A-9B show LILRB1 expression in tumor antigen (OVA)-specific CD8+ T cells. FIG. 9A is a representative FACS plot showing LILRB1 expression on MC38.OVA tumor infitrating CD8+ T cells that were stained positive for OVA tetramer. FIG. 9B shows LILRB1 level in OVA-specific CD8+ T cells in tumor or spleen from MC38.OVA tumor-bearing mice (n=3). *p<0.05 by paired students' t-test.

FIG. 10 shows that anti-PD1 increased LILRB1+ effector CD8 T cells in tumor. Tumor effector-like CD8+ T cells (Teff-like) were gated as PD1-CD62L SLAMF7+CX3CR1+ and memory precursor-like CD8 T cells (Tm-precursor-like) were gated as PD1 CD62L-SLAMF7+CX3CR1. LILRB1 expression was determined by FACS analysis (n=5-8). *p<0.05 by students' t-test.

### DETAILED DESCRIPTION OF THE INVENTION

### 1. Overview

As disclosed and exemplified herein, the inventors discovered that the combination of (i) a PD-1 pathway inhibitor and (ii) a LILRB1/2 pathway inhibitor is surprisingly effective in increasing T-cell mediated cellular cytotoxicity against cancer cells, thereby providing a novel combination therapy for cancer treatment.

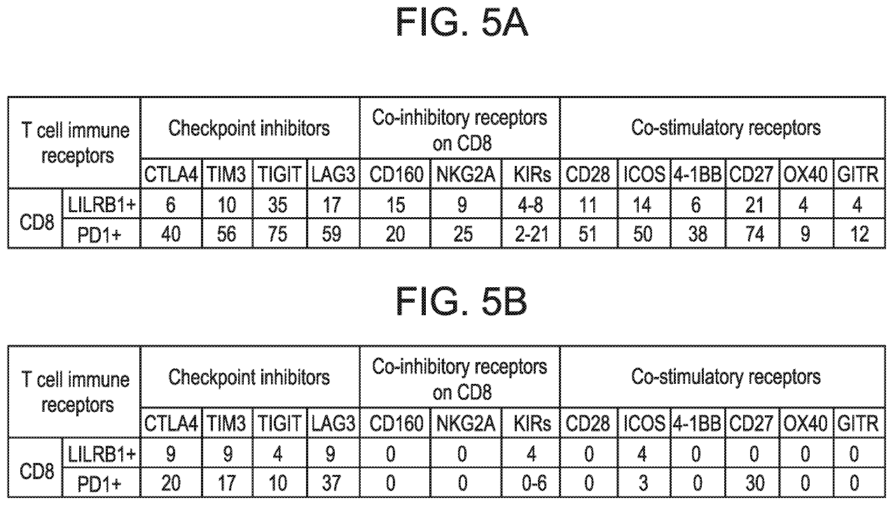

A number of immune checkpoint proteins, such as 4-1BB and CTLA4, have been targeted in combination of PD-1. However, as disclosed and exemplified herein, these immune checkpoint proteins are generally co-expressed with PD-1 on the same population of dysfunctional T cells (see, e.g., FIGS. 5A-5B). Therefore, such combination therapy would be expected to modulate two different pathways on the same T cell. In general, combination therapy targeting two different pathways functioning on the same cell has the advantage of overcoming biological redundancy and avoiding the cancer cell developing drug resistance through mutation or upregulating expression of a single signaling pathway.

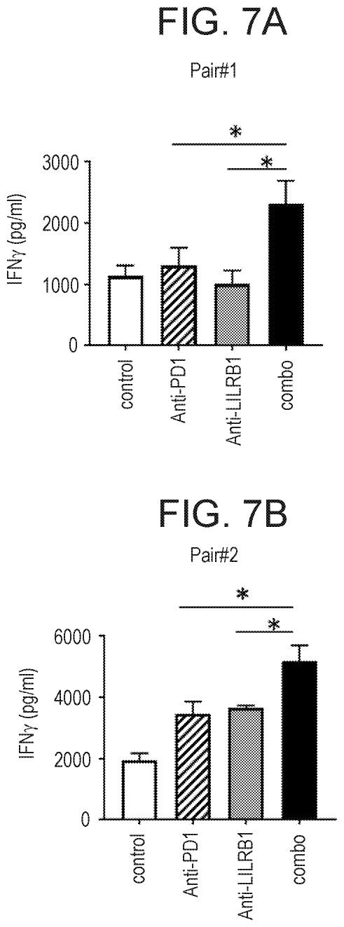

In contrast, the inventors discovered that the expression of PD-1 and LILRB1 is mutually exclusive. PD-1 is highly expressed in “effector memory T cells” (Tem), whereas LILRB1 is highly expressed in “effector T cells” (Teff). Therefore, the combination of anti-PD-1 and anti-LILRB1 targets two different subtypes of CD8+ T cells, thereby mobilizing two different T cell subpopulations and significantly enhancing T-cell expression of effector cytokines, such as IFN-γ, and T-cell dependent cellular cytotoxicity against tumor cell targets. The combination is particularly effective in patients who may respond to anti-PD-1 treatment poorly. For certain patients (see, e.g., FIG. 7, Pair #1), mono-therapy using either anti-PD-1, or anti-LILRB1 failed to improve CD8+ T cell mediated cytotoxicity as compared to control. However, when anti-PD-1 and anti-LILRB1 are combined, the increase in CD8+ T cell cytotoxicity was more than doubled. Such synergistic effect of anti-PD-1 and anti-LILRB1 has not been observed in other combination therapies with PD-1.

Although not wishing to be bound by a particular theory, the inventors believe that by targeting LILBR1/2 signal pathway, the self-renewal of Teff, as well as the life-span of Teff, are both enhanced; by targeting PD-1 signaling pathway, the Tem exhaustion is reduced. The combined effect of targeting both pathways leads to expansion of two subsets of CD8+ T cells, thereby synergistically enhancing CD8-mediated anti-tumor activities.

### 2. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The term “antibody” refers to any form of immunoglobulin molecule that exhibits the desired biological or binding activity. Thus, it is used in the broadest sense and specifically covers, but is not limited to, monoclonal antibodies, polyclonal antibodies, multispecific antibodies (e.g., bispecific antibodies), humanized, fully human antibodies, chimeric antibodies and camelized single domain antibodies.

An “antigen-binding fragment” of an antibody refers to a fragment of a full-length antibody that retains the ability to specifically bind to an antigen (preferably with substantially the same binding affinity). Examples of an antigen-binding fragment includes (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR), disulfide-linked Fvs (dsFv), and anti-idiotypic (anti-Id) antibody and intrabody. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al. Science 242:423-426 (1988) and Huston et al., Proc. Natl. Acad. Sci. USA 85:5879-5883 (1988)). Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al. Proc. Natl. Acad. Sci. USA 90:6444-6448 (1993); Poljak et al., 1994, Structure 2:1121-1123).

“Complementarity Determining Regions” (CDRs) can be identified according to the definitions of the Kabat, Chothia, the accumulation of both Kabat and Chothia, AbM, contact, and/or conformational definitions or any method of CDR determination well known in the art. See, e.g., Kabat et al., 1991, Sequences of Proteins of Immunological Interest, 5th ed. (hypervariable regions); Chothia et al., 1989, Nature 342:877-883 (structural loop structures). AbM definition of CDRs is a compromise between Kabat and Chothia and uses Oxford Molecular's AbM antibody modeling software (Accelrys®). The “contact” definition of CDRs is based on observed antigen contacts, set forth in MacCallum et al., 1996, J. Mol. Biol., 262:732-745. The “conformational” definition of CDRs is based on residues that make enthalpic contributions to antigen binding (see, e.g., Makabe et al., 2008, Journal of Biological Chemistry, 283:1 156-1 166). Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The CDR sequences of the exemplary PD-1 antibody (close 20C1) disclosed herein are defined according to Kabat (see, SEQ ID Nos. 19-24).

The term “treat,” as well as words related thereto, do not necessarily imply 100% or complete cure. Rather, there are varying degrees of treatment of which one of ordinary skill in the art recognizes as having a potential benefit or therapeutic effect. In this respect, the methods of treating cancer of the present disclosure can provide any amount or any level of treatment. Furthermore, the treatment provided by the method of the present disclosure can include treatment of one or more conditions or symptoms or signs of the cancer being treated. Also, the treatment provided by the methods of the present disclosure can encompass slowing the progression of the cancer. For example, the methods can treat cancer by virtue of enhancing the T cell activity or an immune response against the cancer, reducing tumor or cancer growth, reducing metastasis of tumor cells, increasing cell death of tumor or cancer cells, and the like. In exemplary aspects, the methods treat by way of delaying the onset or recurrence of the cancer by 1 day, 2 days, 4 days, 6 days, 8 days, 10 days, 15 days, 30 days, two months, 4 months, 6 months, 1 year, 2 years, 4 years, or more. In exemplary aspects, the methods treat by way increasing the survival of the subject. The term “treatment” also includes prophylactic treatment.

### 3. Combination Therapy Inhibiting PD-1 Pathway and LILRB1/2 Pathway

Disclosed herein are combination therapy to inhibit PD-1 pathway and LILBR1/2 signaling pathway.

In one aspect, the invention provides a method for increasing IFN-γ expression level in a subject that has cancer, comprising administering to the subject a therapeutically effective amount of (i) a first antibody, or antigen-binding fragment thereof, that binds PD-1 or PD-L1; and (ii) a second antibody, or antigen-binding fragment thereof, that binds LILRB1, LILRB2, or HLA-G.

In another aspect, the invention provides a method for increasing IFN-γ expression level in a subject that has cancer, comprising administering to the subject a therapeutically effective amount of (i) a first antibody, or antigen-binding fragment thereof, that binds PD-1 or PD-L1; and (ii) a short interfering RNA (siRNA) that reduces the expression level of HLA-G, as compared to a control.

In another aspect, the invention provides a method for increasing CD8+ T-cell mediated cytotoxicity in a subject that has cancer, comprising administering to the subject a therapeutically effective amount of (i) a first antibody, or antigen-binding fragment thereof, that binds PD-1 or PD-L1; and (ii) a second antibody, or antigen-binding fragment thereof, that binds LILRB1, LILRB2, or HLA-G.

In another aspect, the invention provides a method for increasing CD8+ T-cell mediated cytotoxicity in a subject that has cancer, comprising administering to the subject a therapeutically effective amount of (i) a first antibody, or antigen-binding fragment thereof, that binds PD-1 or PD-L1; and (ii) a short interfering RNA (siRNA) that reduces the expression level of HLA-G, as compared to a control.

In another aspect, the invention provides a method for treating a subject that has a tumor, wherein said subject is refractory to treatment with an anti-PD-1 antibody or antigen-binding fragment thereof, or an anti-PD-L1 antibody or antigen-binding fragment thereof, comprising: administering to the subject a therapeutically effective amount of (i) a first antibody, or antigen-binding fragment thereof, that binds PD-1 or PD-L1; and (ii) a second antibody, or antigen-binding fragment thereof, that binds LILRB1, LILRB2, or HLA-G

In another aspect, the invention provides a method for treating a subject that has a tumor, wherein said subject is refractory to treatment with an anti-PD-1 antibody or antigen-binding fragment thereof, or an anti-PD-L1 antibody or antigen-binding fragment thereof, comprising: administering to the subject a therapeutically effective amount of (i) a first antibody, or antigen-binding fragment thereof, that binds PD-1 or PD-L1; and (ii) a short interfering RNA (siRNA) that reduces the expression level of HLA-G, as compared to a control.

Also provided herein is a composition for use in increasing IFN-γ expression level in a subject that has cancer, said composition comprises (i) a first antibody, or antigen-binding fragment thereof, that binds PD-1 or PD-L1; and (ii) a second antibody, or antigen-binding fragment thereof, that binds LILRB1, LILRB2, or HLA-G.

Also provided herein is a composition for use in increasing IFN-γ expression level in a subject that has cancer, said composition comprises (i) a first antibody, or antigen-binding fragment thereof, that binds PD-1 or PD-L1; and (ii) a short interfering RNA (siRNA) that reduces the expression level of HLA-G, as compared to a control.

Also provided herein is a composition for use in increasing CD8+ T-cell mediated cytotoxicity in a subject that has cancer, said composition comprises (i) a first antibody, or antigen-binding fragment thereof, that binds PD-1 or PD-L1; and (ii) a second antibody, or antigen-binding fragment thereof, that binds LILRB1, LILRB2, or HLA-G.

Also provided herein is a composition for use in increasing CD8+ T-cell mediated cytotoxicity in a subject that has cancer, said composition comprises (i) a first antibody, or antigen-binding fragment thereof, that binds PD-1 or PD-L1; and (ii) a short interfering RNA (siRNA) that reduces the expression level of HLA-G, as compared to a control.

Also provided herein is a composition for use in treating a subject that has a tumor, wherein said subject is refractory to treatment with an anti-PD-1 antibody or antigen-binding fragment thereof, or an anti-PD-L1 antibody or antigen-binding fragment thereof, and wherein said composition comprises (i) a first antibody, or antigen-binding fragment thereof, that binds PD-1 or PD-L1; and (ii) a second antibody, or antigen-binding fragment thereof, that binds LILRB1, LILRB2, or HLA-G.

Also provided herein is a composition for use in treating a subject that has a tumor, wherein said subject is refractory to treatment with an anti-PD-1 antibody or antigen-binding fragment thereof, or an anti-PD-L1 antibody or antigen-binding fragment thereof, and wherein said composition comprises (i) a first antibody, or antigen-binding fragment thereof, that binds PD-1 or PD-L1; and (ii) a short interfering RNA (siRNA) that reduces the expression level of HLA-G, as compared to a control.

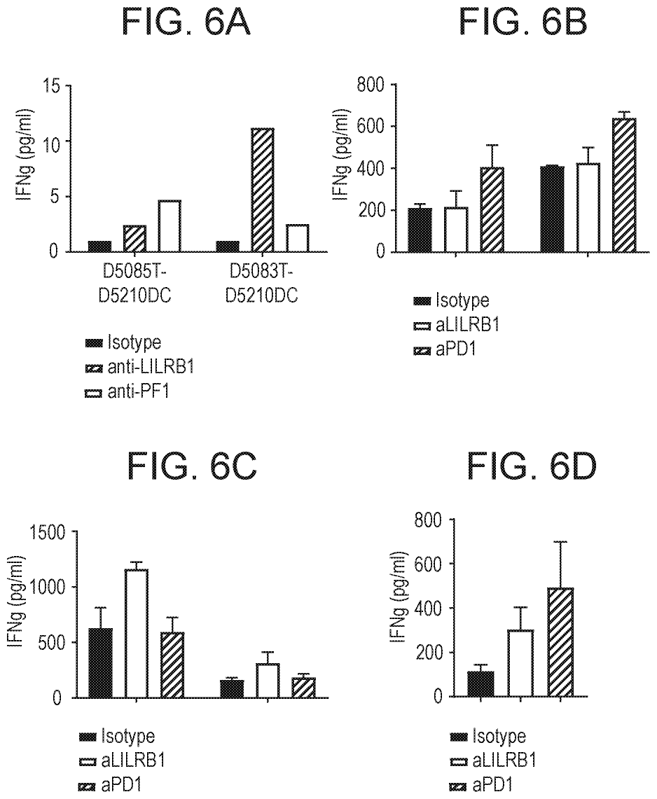

Also provided herein is a method for treating a subject that has a tumor, wherein said subject is refractory to treatment with an anti-PD-1 antibody or antigen-binding fragment thereof, or with an anti-PD-L1 antibody or antigen-binding fragment thereof, comprising: administering to the subject a therapeutically effective amount of an antibody, or antigen-binding fragment thereof, that binds LILRB1, LILRB2, or HLA-G. As exemplified herein, for cancer patients who are refractory to treatment with PD-1 pathway inhibitor, many are responsible to monotherapy using a LILRB1/2 pathway inhibitor (FIGS. 6A-6D). Therefore, the LILRB1/2 pathway inhibitors and their uses described herein are applicable to both monotherapy and combination therapy.

### 3.1 PD-1 Pathway Inhibitors

Programmed cell death protein 1, also known as PD-1 or CD279 (cluster of differentiation 279), is a cell surface receptor that plays an important role in down-regulating the immune system and promoting self-tolerance by suppressing T cell inflammatory activity. PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells). Through these mechanisms, PD-1 inhibits the immune system. This prevents autoimmune diseases, but it can also prevent the immune system from killing cancer cells.

A new class of drugs that block PD-1 pathway (PD-1 pathway inhibitors) can activate the immune system to attack tumors, and are therefore used with varying success to treat some types of cancer. The PD-1 protein in humans is encoded by the PDCD1 gene. PD-1 is a cell surface receptor that belongs to the immunoglobulin superfamily and is expressed on T cells and pro-B cells. PD-1 binds two ligands, PD-L1 and PD-L2.

PD-1 pathway inhibitors disclosed herein include an antibody, or antigen binding fragment thereof, which specifically binds to PD-1 or PD-L1, in particular human PD-1 or human PD-L1. The antibody may be a human antibody, a humanized antibody, or a chimeric antibody. In some embodiments, the constant region of the antibody comprises the constant region from human IgG1, IgG2, IgG3, or IgG4. The antigen binding fragment maybe, for example, Fab, Fab′-SH, F(ab′)2, scFv and Fv fragments.

In certain embodiments, the antibody, or antigen-binding fragment thereof, binds to PD-1, such as human PD-1. Examples of antibodies that bind to human PD-1, are described, e.g, in U.S. Pat. Nos. 7,488,802, 7,521,051, 8,008,449, 8,354,509, 8,168,757, WO2004/004771, WO2004/072286, WO2004/056875, and US2011/0271358. Specific anti-human PD-1 antibodies useful for the invention described herein include, for example: K-3945 (Pembrolizumab, Keytruda®; U.S. Pat. No. 8,952,136); M-3475, a humanized IgG4 mAb with the structure described in WHO Drug Information, Vol. 27, No. 2, pages 161-162 (2013); nivolumab (BMS-936558), a human IgG4 mAb with the structure described in WHO Drug Information, Vol. 27, No. 1, pages 68-69 (2013); the humanized antibodies h409A11, h409A16 and h409A17, which are described in WO2008/156712; AMP-514, which is being developed by MedImmune; humanized antibody CT-011 (Pidilizumab) a monoclonal antibody being developed by Medivation, and anti-PD-1 antibodies disclosed in WO2015/119923 (the heavy and light chains comprise SEQ ID NO: 21 and SEQ ID NO: 22, respectively).

In certain embodiments, the anti-PD-1 antibody binds to cynomolgus PD-1. In exemplary embodiments, the anti-PD-1 antibody binds to both human PD-1 and cynomolgus PD-1.

In certain embodiments, the antibody, or antigen-binding fragment thereof, binds to PD-L1, such as human PD-L1. Examples of mAbs that bind to human PD-L1 are described, e.g., in WO2013/019906, WO2010/077634 and U.S. Pat. No. 8,383,796. Specific anti-human PD-L1 antibodies useful for the invention described herein include, for example, MPDL3280A (Atezolizumab), MEDI4736 (Durvalumab), SB0010718C (Avelumab), BMS-936559, and an antibody which comprises the heavy chain and light chain variable regions of SEQ ID NO: 24 and SEQ ID NO: 21, respectively, of WO2013/019906.

In certain embodiments, the anti-PD-1 antibody, or antigen-binding fragment thereof, binds to cynomolgus PD-L1. In exemplary embodiments, the anti-PD-L1 antibody, or antigen-binding fragment thereof, binds to both human PD-L1 and cynomolgus PD-L1.

In certain embodiments, the antibody or antigen-binding fragment thereof described herein inhibits the signal transduction resulting from the interaction of PD-1 with one or more of its binding partners, such as PD-L1, or PD-L2. In certain embodiments, the antibody, or antigen-binding fragment thereof, inhibits at least about 40%, at least about 50%, at least about 60%, at least about 70%, at least about 80%, or at least about 90% of the binding interactions between PD-1 and PD-L1 or PD-L2.

In an exemplary embodiment, the invention provides an antibody, or antigen-binding fragment thereof, that specifically binds human PD-1, comprising: (i) a heavy chain variable region (VH) that comprises: a VH complementarity determining region one (CDR-H1) comprising the amino acid sequence of SEQ ID NO: 19, a VH CDR-H2 comprising the amino acid sequence of SEQ ID NO: 20; and a VH CDR-H3 comprising the amino acid sequence of SEQ ID NO: 21; and (ii) a light chain variable region (VL) that comprises: a VL complementarity determining region one (CDR-L1) comprising the amino acid sequence of SEQ ID NO: 22, a VL CDR-L2 comprising the amino acid sequence of SEQ ID NO: 23; and (c) a VL CDR-L3 comprising the amino acid sequence of SEQ ID NO: 24.

In certain embodiments, the antibody, or antigen-binding fragment thereof, described herein comprises the following heavy chain CDR sequences: (i) a CDR-H1 sharing at least 90%, at least 91%, at least 92%, at least 93%, at least 94%, or at least 95% identical to SEQ ID NO:19, a CDR-H2 sharing at least 90%, at least 91%, at least 92%, at least 93%, at least 94%, or at least 95% identity with SEQ ID NO: 20, and a CDR-H3 sharing at least 90%, at least 91%, at least 92%, at least 93%, at least 94%, or at least 95% identity with SEQ ID NO: 21; and/or (ii) the following light chain CDR sequences: a CDR-L1 sharing at least 90%, at least 91%, at least 92%, at least 93%, at least 94%, or at least 95% identity with SEQ ID NO: 22, a CDR-L2 sharing at least 90%, at least 91%, at least 92%, at least 93%, at least 94%, or at least 95% identity with SEQ ID NO: 23, and a CDR-L3 sharing at least 90%, at least 91%, at least 92%, at least 93%, at least 94%, or at least 95% identity with SEQ ID NO: 24.

In certain embodiments, no more than 5, no more than 4, no more than 3, no more than 3, no more than 2, or no more than one substitution is made in the sequence of CDR-L1, relative to SEQ ID NO. 22. In certain embodiments, no more than 4, no more than 3, no more than 3, no more than 2, or no more than one substitution is made in the sequence of CDR-L2, relative to SEQ ID NO. 23. In certain embodiments, no more than 5, no more than 4, no more than 3, no more than 3, no more than 2, or no more than one substitution is made in the sequence of CDR-L3, relative to SEQ ID NO. 24. In certain embodiments, no more than 3, no more than 2, or no more than one substitution is made in the sequence of CDR-H1, relative to SEQ ID NO. 19. In certain embodiments, no more than 10, no more than 9, no more than 8, no more than 7, no more than 6, no more than 5, no more than 4, no more than 3, no more than 3, no more than 2, or no more than one substitution is made in the sequence of CDR-H2, relative to SEQ ID NO. 20. In certain embodiments, no more than 5, no more than 4, no more than 3, no more than 3, no more than 2, or no more than one substitution is made in the sequence of CDR-H3, relative to SEQ ID NO. 21. In certain embodiments, the substitution does not change binding affinity (K_{D}) value by more than 3 orders of magnitude, more than 2 orders of magnitude, or 1 order of magnitude, as compared with the K_{D} of the antibody, or antigen-binding fragment thereof, without the substitution.

In certain embodiments, the antibody or antigen-binding fragment thereof described herein comprises (i) a VH comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 25, and/or (ii) a VL comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 26. Any combination of these VL and VH sequences is also encompassed by the invention

In certain embodiments, the antibody or antigen-binding fragment thereof described herein comprises (i) a CH comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 29; and/or (ii) a CL comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 30. Any combination of these CH and CL sequences is also encompassed by the invention.

In certain embodiments, the antibody or antigen-binding fragment thereof described herein comprises an Fc domain. The Fc domain can be derived from IgA (e.g., IgA1 or IgA2), IgG, IgE, or IgG (e.g., IgG1, IgG2, IgG3, or IgG4).

In certain embodiments, the antibody or antigen-binding fragment thereof described herein comprises (i) a heavy chain comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 27, and/or (ii) a light chain comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 28. Any combination of these heavy chain and light chain sequences is also encompassed by the invention.

Sometimes, the C-terminal lysine of an antibody heavy chain undergoes cleavage by carboxypeptidase during expression. Accordingly, in some aspects, the anti-PD-1 antibody comprises a heavy chain constant region lacking the C-terminal Lys. A heavy chain constant region lacking the C-terminal Lys advantageously prevents carboxypeptidase to act on the heavy chain of the anti-PD-1 antibody.

In certain embodiments, the anti-PD-1 antibody, antigen binding antibody fragment thereof, or anti-PD-1 antibody protein product comprises one or more amino acid modifications, relative to the naturally-occurring counterpart, in order to improve half-life/stability or to render the antibody more suitable for expression/manufacturability. In exemplary embodiments, the anti-PD-1 antibody is designed to prevent or reduce interaction between the anti-PD-1 antibody and Fc receptors. In exemplary instances, the anti-PD-1 antibody is a Stable Effector Functionless (SEFL) antibody comprising a constant region that lacks the ability to interact with Fcγ receptors. SEFL antibodies are known in the art. See, e.g., Liu et al., J Biol Chem 292:1876-1883 (2016); and Jacobsen et al., J. Biol. Chem. 292:1865-1875 (2017). In exemplary aspects, the SEFL antibody comprises one or more of the following mutations, numbered according to the EU system: L242C, A287C, R292C, N297G, V302C, L306C, and/or K334C. In exemplary aspects, the SEFL antibody comprises N297G. In exemplary aspects, the SEFL antibody comprises A287C, N297G, and L306C. In other exemplary aspects, the SEFL antibody comprises R292C, N297G, and V302C (i.e., SEFL2-2).

Additional antibodies, or antigen-binding fragment thereof, of the invention can be obtained using PD-1 or PD-L1 (or antigenic fragment thereof) as an antigen. Exemplary sequences of human PD-1 and PD-L1 are provided herein as SEQ ID Nos. 1 and 2, respectively. Using these exemplary sequences as antigens, antibodies (or antigen-binding fragments thereof) of the invention can be obtained using art-known methods. For example, monoclonal antibodies may be made by the hybridoma method, or may be made by recombinant DNA methods. The antibodies may also be isolated from phage antibody libraries.

In certain embodiments, the antibody, or antigen-binding fragment thereof, of the invention binds to SEQ ID NO:1, or an antigenic fragment of SEQ ID NO:1. In certain embodiments, the antibody, or antigen-binding fragment thereof, of the invention binds to an amino acid sequence that is at least 70%, at least 75%, at least 80%, at least 85%, at least 90%, at least 95%, at least 97%, or at least 99% identical to SEQ ID NO:1, or an antigenic fragment of SEQ ID NO:1.

In certain embodiments, the antibody, or antigen-binding fragment thereof, of the invention binds to SEQ ID NO:2, or an antigenic fragment of SEQ ID NO:2. In certain embodiments, the antibody, or antigen-binding fragment thereof, of the invention binds to an amino acid sequence that is at least 70%, at least 75%, at least 80%, at least 85%, at least 90%, at least 95%, at least 97%, or at least 99% identical to SEQ ID NO:2, or an antigenic fragment of SEQ ID NO:2.

Other PD-1 pathway inhibitors that are useful for the invention include, for example, an immunoadhesin that specifically binds to PD-1 or PD-L1, a fusion protein comprising the extracellular or PD-1 binding portion of PD-L1 or PD-L2 fused to a constant region, such as an Fc region of an immunoglobulin molecule. Examples of immunoadhesion molecules that specifically bind to PD-1 are described in WO2010/027827 and WO201 1/066342. Examples of fusion proteins that are useful as PD-1 pathway inhibitors include AMP-224 (also known as B7-DClg), which is a PD-L2-Fc fusion protein and binds to human PD-1.

Antisense RNA, or small interfering RNA (siRNA) may also be used as PD-1 pathway inhibitors. Various art-known methods can be used to analyze the sequence of PD-1 and PD-L1 (such as SEQ ID Nos. 1 and 2) to obtain antisense RNA or siRNA that inhibits the expression of PD-1 and/or PD-L1. Several siRNA design algorithms have emerged recently to offer high success rates for silencing human genes. Examples of such algorithms include, e.g., Cenix algorithm, and GeneLink RNAi Explorer. General parameters for siRNA design strategies may include, for example, (1) low to medium GC content (30-50%); (2) absence of internal repeats or palindromes; (3) presence of an A at position 3 of the sense strand; (4) presence of A at position 19 of the sense strand; (5) absence of G or C at position 19 of the sense strand; (6) presence of U at position 10 of the sense strand; (7) absence of a G at position 13 of the sense strand; and/or (8) at least 3 A/Us at positions 15-19 of the sense strand.

In some embodiments, the PD-1 pathway inhibitor described herein reduces the negative co-stimulatory signal mediated by or through cell surface proteins expressed on T lymphocytes mediated signaling through PD-1, so to render a dysfunctional T-cell less dysfunctional (e.g., enhancing effector responses to antigen recognition).

### 3.2. LILRB1/2 Pathway Inhibitors

The leukocyte immunoglobulin-like receptors (LILR) are a family of receptors possessing extracellular immunoglobulin domains. They are also known as CD85, ILTs and LIR, and can exert immunomodulatory effects on a wide range of immune cells. The human genes encoding these receptors are found in a gene cluster at chromosomal region 19q13.4. They include, LILRA1, LILRA2, LILRA3, LILRA4, LILRA5, LILRA6, LILRB1, LILRB2, LILRB3, LILRB4, LILRB5, LILRB6 or LILRA6, and LILRB7 or LILRA5. A subset of LILRs recognize MHC class I molecules (also known as HLA class I in humans). Of these, the inhibitory receptors LILRB1 and LILRB2 show a broad specificity for classical and non-classical MHC alleles with preferential binding to b2m-associated complexes. In contrast, the activating receptors LILRA1 and LILRA3 prefer b2m-independent free heavy chains of MHC class I, and in particular HLA-C alleles.

Leukocyte immunoglobulin-like receptor subfamily B member 1 (LILRB1) is a protein that in humans is encoded by the LILRB1 gene. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on immune cells where it binds to MHC class I molecules on antigen-presenting cells and transduces a negative signal that inhibits stimulation of an immune response. LILRB1 was also reported to be expressed in human gastric cancer cells and may enhance tumor growth. It is thought to control inflammatory responses and cytotoxicity to help focus the immune response and limit autoreactivity. Multiple transcript variants encoding different isoforms have been found for this gene. LILRB1 is also known as ILT2, ILT-2, CD85, CD85J, LIR1, LIR-1, and MIR7. The human LILRB1 protein have several different isoforms. Examples of such isoforms are provided herein as SEQ ID Nos. 3-7. These sequences are not exhaustive, and a skilled artisan would know that other isoforms and polymorphisms exist, and that their sequences are readily available from public databases.

Leukocyte immunoglobulin-like receptor subfamily B member 2 (LILRB2) is a protein that in humans is encoded by the LILRB2 gene. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on immune cells where it binds to MHC class I molecules on antigen-presenting cells and transduces a negative signal that inhibits stimulation of an immune response. It is thought to control inflammatory responses and cytotoxicity to help focus the immune response and limit autoreactivity. The receptor is also expressed on human non-small cell lung cancer cells (see Sun et al., Chest. 2008, 134:783-788). Multiple transcript variants encoding different isoforms have been found for this gene. LILRB2 has been shown to interact with PTPN6. The human LILRB2 protein also have several different isoforms. Examples of such isoforms are provided herein as SEQ ID Nos. 8-11. These sequences are not exhaustive, and a skilled artisan would know that other isoforms and polymorphisms exist, and that their sequences are readily available from public databases.

A major histocompatibility complex (MHC) Class I complex is made of human leukocyte antigen (HLA) alpha chains and a beta-2-macroglobulin protein (B2M) that assemble to form a complex. HLA alpha chains in humans include the alpha chains HLA-A, HLA-B, HLA-C, HLA-E, HLA-F, HLA-G, HLA-K, and HLA-L. The HLA-A, HLA-B, and HLA-C alpha chains are typically referred to as classical HLA alpha chains. Non-classical MHC Class I HLA alpha chains include HLA-E, HLA-F, HLA-G, HLA-K, and HLA-L.

HLA-G is encoded by a nonclassical class Ib gene, and HLA-G protein possesses some unusual characteristics, including restricted expression, limited number of polymorphisms, and alternatively spliced mRNA variants. The genomic structure of HLA-G is similar to other class I genes, however HLA-G is unique in several other respects. The HLA-G gene has eight exons encoding a signal peptide (exon 1), the a1, a2, and a3 domains (exons 2, 3, and 4, respectively), the transmembrane domain (exon 5) and the intracellular domain (exons 6 and 7). The premature stop codon in exon 6 is responsible in slower turnover, prolonged expression of HLA-G at the cell surface, and the inefficient presentation of exogenous peptides (Park, B., et al., Immunity, 15:213 (2001)). Another unique future of HLA-G is that it encodes at least seven isoforms as a result of alternative splicing (HLA-G1 to HLA-G7). The full-length membrane-bound isoforms, HLA-G1, is structurally similar to other class I genes, except for the truncated cytoplasmic tail. A stop sequence in intron 4 results in two soluble isoforms, HLA-G5 and HLA-G6.

HLA-G exists in numerous isoforms and polymorphisms. Examples of some HLA-G sequences are provided herein as SEQ ID Nos. 12-18. These sequences are not exhaustive, and a skilled artisan would know that other isoforms and polymorphisms exist, and that their sequences are readily available from public databases. For example, the following GenBank Accession Nos. refer to HLA-G: NM_002127.5, XM_006715080.1, XM_006725041.1, XM_006725700.1, and XM_006725909.1.

As described herein, the inventors believe that the LILRB1/2 pathway inhibits immune cell function. Through the interaction between LILRB1/2 and HLA-G, a number of immune cell functions are suppressed, including, for example: NK/CD8 cell mediated cytolytic activity, DC cell maturation and IL-12 production, macrophage phagocytosis, and B cell proliferation and antibody production. Accordingly, inhibiting LILRB1/2 pathway can be an attractive mechanism for cancer treatment.

LILRB1/2 pathway inhibitors disclosed herein include an antibody, or antigen binding fragment thereof, which specifically binds to LILRB1, LILRB2, or HLA-G, in particular human LILRB1, human LILRB2, or human HLA-G. The antibody may be a human antibody, a humanized antibody, or a chimeric antibody. In some embodiments, the constant region of the antibody comprises the constant region from human IgG1, IgG2, IgG3, or IgG4. The antigen binding fragment maybe, for example, Fab, Fab′-SH, F(ab′)2, scFv and Fv fragments.

In certain embodiments, the antibody, or antigen-binding fragment thereof, binds to LILRB1, such as human LILRB1. Examples of anti-LILRB1 antibodies include, e.g., Human LILRB1/CD85j/ILT2 antibody clones MAB20171 and MAB20172 (available from R&D Systems); anti-LILRB1 clone 3D3-1D12 (available from Sigma-Aldrich); anti-LILRB1 clone GH1/75 (available from Novus Biologicals); and anti-LILRB4 antibodies that also cross-react with LILRB1, as described in US2018/0086829. These antibodies are from non-human species. For treatment of human subjects, it would be describable to further humanize these antibodies, using commonly-known methods in the art, such as CDR grafting.

In certain embodiments, the antibody, or antigen-binding fragment thereof, binds to LILRB2, such as human LILRB2. Examples of anti-LILRB2 antibodies. Examples of anti-LILRB2 antibodies include, e.g., Human LILRB2/CD85d/ILT4 antibody clone MAB2078 (available from R&D Systems); anti-LILRB2 clone 1D4 (available from Sigma-Aldrich); and anti-LILRB4 antibodies that also cross-react with LILRB1, as described in US2018/0086829. Again, for treatment of human subjects, it would be describable to further humanize these non-human antibodies using commonly-known methods in the art, such as CDR grafting.

In certain embodiments, the antibody, or antigen-binding fragment thereof, binds both LILRB1 and LILRB2. In certain embodiments, the antibody, or antigen-binding fragment thereof, binds both human LILRB1 and human LILRB2.

In certain embodiments, the antibody, or antigen-binding fragment thereof, binds to HLA-G, such as human HLA-G. Examples of anti-HLA-G antibodies can be found, e.g., in WO 2017/207775 and WO2014/072534.

In certain, the antibody described here inhibits the signal transduction resulting from the interaction of LILRB1/LILBR2 with one or more of its binding partners, such as HLA-G. In certain embodiments, the antibody inhibits at least about 40%, at least about 50%, at least about 60%, at least about 70%, at least about 80%, or at least about 90% of the binding interactions between LILRB1 and HLA-G, or between LILRB2 and HLA-G.

In an exemplary embodiment, the invention provides an antibody, or antigen-binding fragment thereof, that specifically binds human LILRB1, comprising: (i) a heavy chain variable region (VH) that comprises: a VH complementarity determining region one (CDR-H1) comprising the amino acid sequence of SEQ ID NO: 36, a VH CDR-H2 comprising the amino acid sequence of SEQ ID NO: 37; and a VH CDR-H3 comprising the amino acid sequence of SEQ ID NO: 38; and (ii) a light chain variable region (VL) that comprises: a VL complementarity determining region one (CDR-L1) comprising the amino acid sequence of SEQ ID NO: 39, a VL CDR-L2 comprising the amino acid sequence of SEQ ID NO: 40; and (c) a VL CDR-L3 comprising the amino acid sequence of SEQ ID NO: 41.

In certain embodiments, the antibody, or antigen-binding fragment thereof, described herein comprises the following heavy chain CDR sequences: (i) a CDR-H1 sharing at least 90%, at least 91%, at least 92%, at least 93%, at least 94%, or at least 95% identical to SEQ ID NO:36, a CDR-H2 sharing at least 90%, at least 91%, at least 92%, at least 93%, at least 94%, or at least 95% identity with SEQ ID NO: 37, and a CDR-H3 sharing at least 90%, at least 91%, at least 92%, at least 93%, at least 94%, or at least 95% identity with SEQ ID NO: 38; and/or (ii) the following light chain CDR sequences: a CDR-L1 sharing at least 90%, at least 91%, at least 92%, at least 93%, at least 94%, or at least 95% identity with SEQ ID NO: 39, a CDR-L2 sharing at least 90%, at least 91%, at least 92%, at least 93%, at least 94%, or at least 95% identity with SEQ ID NO: 40, and a CDR-L3 sharing at least 90%, at least 91%, at least 92%, at least 93%, at least 94%, or at least 95% identity with SEQ ID NO: 41.

In certain embodiments, no more than 5, no more than 4, no more than 3, no more than 3, no more than 2, or no more than one substitution is made in the sequence of CDR-L1, relative to SEQ ID NO. 39. In certain embodiments, no more than 4, no more than 3, no more than 3, no more than 2, or no more than one substitution is made in the sequence of CDR-L2, relative to SEQ ID NO. 40. In certain embodiments, no more than 5, no more than 4, no more than 3, no more than 3, no more than 2, or no more than one substitution is made in the sequence of CDR-L3, relative to SEQ ID NO. 41. In certain embodiments, no more than 3, no more than 2, or no more than one substitution is made in the sequence of CDR-H1, relative to SEQ ID NO. 36. In certain embodiments, no more than 10, no more than 9, no more than 8, no more than 7, no more than 6, no more than 5, no more than 4, no more than 3, no more than 3, no more than 2, or no more than one substitution is made in the sequence of CDR-H2, relative to SEQ ID NO. 37. In certain embodiments, no more than 5, no more than 4, no more than 3, no more than 3, no more than 2, or no more than one substitution is made in the sequence of CDR-H3, relative to SEQ ID NO. 38. In certain embodiments, the substitution does not change binding affinity (K_{D}) value by more than 3 orders of magnitude, more than 2 orders of magnitude, or 1 order of magnitude, as compared with the K_{D} of the antibody, or antigen-binding fragment thereof, without the substitution.

In certain embodiments, the antibody or antigen-binding fragment thereof described herein comprises (i) a VH comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 42, and/or (ii) a VL comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 43. Any combination of these VL and VH sequences is also encompassed by the invention.

In certain embodiments, the antibody or antigen-binding fragment thereof described herein comprises a human immunoglobulin kappa constant domain sequence (e.g., SEQ ID NO:30). In certain embodiments, the antibody or antigen-binding fragment thereof described herein comprises a human immunoglobulin lambda constant domain sequence (e.g., SEQ ID NO:47).

In certain embodiments, the antibody or antigen-binding fragment thereof described herein comprises (i) a CH comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 46; and/or (ii) a CL comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 47. Any combination of these CH and CL sequences is also encompassed by the invention.

In certain embodiments, the antibody or antigen-binding fragment thereof described herein comprises an Fc domain. The Fc domain can be derived from IgA (e.g., IgA1 or IgA2), IgG, IgE, or IgG (e.g., IgG1, IgG2, IgG3, or IgG4).

In certain embodiments, the antibody or antigen-binding fragment thereof described herein comprises a human immunoglobulin heavy constant gamma 4 (IgG4) sequence (e.g., SEQ ID NO: 46). In certain embodiments, the antibody or antigen-binding fragment thereof described herein comprises a human immunoglobulin heavy constant gamma 1 (IgG1) sequence (e.g., SEQ ID NO: 68). In certain embodiments, the antibody or antigen-binding fragment thereof described herein comprises a human immunoglobulin heavy constant gamma 2 (IgG2) sequence (e.g., SEQ ID NO: 69). In certain embodiments, the antibody or antigen-binding fragment thereof described herein comprises a human immunoglobulin heavy constant gamma 3 (IgG3) sequence (e.g., SEQ ID NO: 70).

In certain embodiments, the antibody or antigen-binding fragment thereof described herein comprises (i) a heavy chain comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 44, and/or (ii) a light chain comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 45. Any combination of these heavy chain and light chain sequences is also encompassed by the invention.

In an exemplary embodiment, the invention provides an antibody, or antigen-binding fragment thereof, that specifically binds human LILRB1, comprising: (i) a heavy chain variable region (VH) that comprises: a VH complementarity determining region one (CDR-H1) comprising the amino acid sequence of SEQ ID NO: 48, a VH CDR-H2 comprising the amino acid sequence of SEQ ID NO: 49; and a VH CDR-H3 comprising the amino acid sequence of SEQ ID NO: 50; and (ii) a light chain variable region (VL) that comprises: a VL complementarity determining region one (CDR-L1) comprising the amino acid sequence of SEQ ID NO: 51, a VL CDR-L2 comprising the amino acid sequence of SEQ ID NO: 52; and (c) a VL CDR-L3 comprising the amino acid sequence of SEQ ID NO: 53.

In certain embodiments, the antibody, or antigen-binding fragment thereof, described herein comprises the following heavy chain CDR sequences: (i) a CDR-H1 sharing at least 90%, at least 91%, at least 92%, at least 93%, at least 94%, or at least 95% identical to SEQ ID NO:48, a CDR-H2 sharing at least 90%, at least 91%, at least 92%, at least 93%, at least 94%, or at least 95% identity with SEQ ID NO: 49, and a CDR-H3 sharing at least 90%, at least 91%, at least 92%, at least 93%, at least 94%, or at least 95% identity with SEQ ID NO: 50; and/or (ii) the following light chain CDR sequences: a CDR-L1 sharing at least 90%, at least 91%, at least 92%, at least 93%, at least 94%, or at least 95% identity with SEQ ID NO: 51, a CDR-L2 sharing at least 90%, at least 91%, at least 92%, at least 93%, at least 94%, or at least 95% identity with SEQ ID NO: 52, and a CDR-L3 sharing at least 90%, at least 91%, at least 92%, at least 93%, at least 94%, or at least 95% identity with SEQ ID NO: 53.

In certain embodiments, no more than 5, no more than 4, no more than 3, no more than 3, no more than 2, or no more than one substitution is made in the sequence of CDR-L1, relative to SEQ ID NO. 51. In certain embodiments, no more than 4, no more than 3, no more than 3, no more than 2, or no more than one substitution is made in the sequence of CDR-L2, relative to SEQ ID NO. 52. In certain embodiments, no more than 5, no more than 4, no more than 3, no more than 3, no more than 2, or no more than one substitution is made in the sequence of CDR-L3, relative to SEQ ID NO. 53. In certain embodiments, no more than 3, no more than 2, or no more than one substitution is made in the sequence of CDR-H1, relative to SEQ ID NO. 48. In certain embodiments, no more than 10, no more than 9, no more than 8, no more than 7, no more than 6, no more than 5, no more than 4, no more than 3, no more than 3, no more than 2, or no more than one substitution is made in the sequence of CDR-H2, relative to SEQ ID NO. 49. In certain embodiments, no more than 5, no more than 4, no more than 3, no more than 3, no more than 2, or no more than one substitution is made in the sequence of CDR-H3, relative to SEQ ID NO. 50. In certain embodiments, the substitution does not change binding affinity (K_{D}) value by more than 3 orders of magnitude, more than 2 orders of magnitude, or 1 order of magnitude, as compared with the K_{D} of the antibody, or antigen-binding fragment thereof, without the substitution.

In certain embodiments, the antibody or antigen-binding fragment thereof described herein comprises (i) a VH comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 54, and/or (ii) a VL comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 55. Any combination of these VL and VH sequences is also encompassed by the invention.

In certain embodiments, the antibody or antigen-binding fragment thereof described herein comprises a human immunoglobulin kappa constant domain sequence (e.g., SEQ ID NO:30). In certain embodiments, the antibody or antigen-binding fragment thereof described herein comprises a human immunoglobulin lambda constant domain sequence (e.g., SEQ ID NO:47).

In certain embodiments, the antibody or antigen-binding fragment thereof described herein comprises (i) a CH comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 46; and/or (ii) a CL comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 30. Any combination of these CH and CL sequences is also encompassed by the invention.

In certain embodiments, the antibody or antigen-binding fragment thereof described herein comprises an Fc domain. The Fc domain can be derived from IgA (e.g., IgA1 or IgA2), IgG, IgE, or IgG (e.g., IgG1, IgG2, IgG3, or IgG4).

In certain embodiments, the antibody or antigen-binding fragment thereof described herein comprises a human immunoglobulin heavy constant gamma 4 (IgG4) sequence (e.g., SEQ ID NO: 46). In certain embodiments, the antibody or antigen-binding fragment thereof described herein comprises a human immunoglobulin heavy constant gamma 1 (IgG1) sequence (e.g., SEQ ID NO: 68). In certain embodiments, the antibody or antigen-binding fragment thereof described herein comprises a human immunoglobulin heavy constant gamma 2 (IgG2) sequence (e.g., SEQ ID NO: 69). In certain embodiments, the antibody or antigen-binding fragment thereof described herein comprises a human immunoglobulin heavy constant gamma 3 (IgG3) sequence (e.g., SEQ ID NO: 70).

In certain embodiments, the antibody or antigen-binding fragment thereof described herein comprises (i) a heavy chain comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 56, and/or (ii) a light chain comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 57. Any combination of these heavy chain and light chain sequences is also encompassed by the invention.

In an exemplary embodiment, the invention provides an antibody, or antigen-binding fragment thereof, that specifically binds human LILRB1, comprising: (i) a heavy chain variable region (VH) that comprises: a VH complementarity determining region one (CDR-H1) comprising the amino acid sequence of SEQ ID NO: 58, a VH CDR-H2 comprising the amino acid sequence of SEQ ID NO: 59; and a VH CDR-H3 comprising the amino acid sequence of SEQ ID NO: 60; and (ii) a light chain variable region (VL) that comprises: a VL complementarity determining region one (CDR-L1) comprising the amino acid sequence of SEQ ID NO: 61, a VL CDR-L2 comprising the amino acid sequence of SEQ ID NO: 62; and (c) a VL CDR-L3 comprising the amino acid sequence of SEQ ID NO: 63.

In certain embodiments, the antibody, or antigen-binding fragment thereof, described herein comprises the following heavy chain CDR sequences: (i) a CDR-H1 sharing at least 90%, at least 91%, at least 92%, at least 93%, at least 94%, or at least 95% identical to SEQ ID NO:58, a CDR-H2 sharing at least 90%, at least 91%, at least 92%, at least 93%, at least 94%, or at least 95% identity with SEQ ID NO: 59, and a CDR-H3 sharing at least 90%, at least 91%, at least 92%, at least 93%, at least 94%, or at least 95% identity with SEQ ID NO: 60; and/or (ii) the following light chain CDR sequences: a CDR-L1 sharing at least 90%, at least 91%, at least 92%, at least 93%, at least 94%, or at least 95% identity with SEQ ID NO: 61, a CDR-L2 sharing at least 90%, at least 91%, at least 92%, at least 93%, at least 94%, or at least 95% identity with SEQ ID NO: 62, and a CDR-L3 sharing at least 90%, at least 91%, at least 92%, at least 93%, at least 94%, or at least 95% identity with SEQ ID NO: 63.

In certain embodiments, no more than 5, no more than 4, no more than 3, no more than 3, no more than 2, or no more than one substitution is made in the sequence of CDR-L1, relative to SEQ ID NO. 61. In certain embodiments, no more than 4, no more than 3, no more than 3, no more than 2, or no more than one substitution is made in the sequence of CDR-L2, relative to SEQ ID NO. 62. In certain embodiments, no more than 5, no more than 4, no more than 3, no more than 3, no more than 2, or no more than one substitution is made in the sequence of CDR-L3, relative to SEQ ID NO. 63. In certain embodiments, no more than 3, no more than 2, or no more than one substitution is made in the sequence of CDR-H1, relative to SEQ ID NO. 58. In certain embodiments, no more than 10, no more than 9, no more than 8, no more than 7, no more than 6, no more than 5, no more than 4, no more than 3, no more than 3, no more than 2, or no more than one substitution is made in the sequence of CDR-H2, relative to SEQ ID NO. 59. In certain embodiments, no more than 5, no more than 4, no more than 3, no more than 3, no more than 2, or no more than one substitution is made in the sequence of CDR-H3, relative to SEQ ID NO. 60. In certain embodiments, the substitution does not change binding affinity (K_{D}) value by more than 3 orders of magnitude, more than 2 orders of magnitude, or 1 order of magnitude, as compared with the K_{D} of the antibody, or antigen-binding fragment thereof, without the substitution.

In certain embodiments, the antibody or antigen-binding fragment thereof described herein comprises (i) a VH comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 64, and/or (ii) a VL comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 65. Any combination of these VL and VH sequences is also encompassed by the invention.

In certain embodiments, the antibody or antigen-binding fragment thereof described herein comprises a human immunoglobulin kappa constant domain sequence (e.g., SEQ ID NO:30). In certain embodiments, the antibody or antigen-binding fragment thereof described herein comprises a human immunoglobulin lambda constant domain sequence (e.g., SEQ ID NO:47).

In certain embodiments, the antibody or antigen-binding fragment thereof described herein comprises (i) a CH comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 46; and/or (ii) a CL comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 47. Any combination of these CH and CL sequences is also encompassed by the invention.

In certain embodiments, the antibody or antigen-binding fragment thereof described herein comprises an Fc domain. The Fc domain can be derived from IgA (e.g., IgA1 or IgA2), IgG, IgE, or IgG (e.g., IgG1, IgG2, IgG3, or IgG4).

In certain embodiments, the antibody or antigen-binding fragment thereof described herein comprises a human immunoglobulin heavy constant gamma 4 (IgG4) sequence (e.g., SEQ ID NO: 46). In certain embodiments, the antibody or antigen-binding fragment thereof described herein comprises a human immunoglobulin heavy constant gamma 1 (IgG1) sequence (e.g., SEQ ID NO: 68). In certain embodiments, the antibody or antigen-binding fragment thereof described herein comprises a human immunoglobulin heavy constant gamma 2 (IgG2) sequence (e.g., SEQ ID NO: 69). In certain embodiments, the antibody or antigen-binding fragment thereof described herein comprises a human immunoglobulin heavy constant gamma 3 (IgG3) sequence (e.g., SEQ ID NO: 70).

In certain embodiments, the antibody or antigen-binding fragment thereof described herein comprises (i) a heavy chain comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 66, and/or (ii) a light chain comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 67. Any combination of these heavy chain and light chain sequences is also encompassed by the invention.

Sometimes, the C-terminal lysine of an antibody heavy chain undergoes cleavage by carboxypeptidase during expression. Accordingly, in some aspects, the anti-LILRB1 antibody comprises a heavy chain constant region lacking the C-terminal Lys. A heavy chain constant region lacking the C-terminal Lys advantageously prevents carboxypeptidase to act on the heavy chain of the anti-LILRB1 antibody.

In certain embodiments, the anti-LILRB1 antibody, antigen binding antibody fragment thereof, or anti-LILRB1 antibody protein product comprises one or more amino acid modifications, relative to the naturally-occurring counterpart, in order to improve half-life/stability or to render the antibody more suitable for expression/manufacturability. In exemplary embodiments, the anti-LILRB1 antibody is designed to prevent or reduce interaction between the anti-LILRB1 antibody and Fc receptors. In exemplary instances, the anti-LILRB1 antibody is a Stable Effector Functionless (SEFL) antibody comprising a constant region that lacks the ability to interact with Fcγ receptors. SEFL antibodies are known in the art. See, e.g., Liu et al., J Biol Chem 292:1876-1883 (2016); and Jacobsen et al., J. Biol. Chem. 292:1865-1875 (2017). In exemplary aspects, the SEFL antibody comprises one or more of the following mutations, numbered according to the EU system: L242C, A287C, R292C, N297G, V302C, L306C, and/or K334C. In exemplary aspects, the SEFL antibody comprises N297G. In exemplary aspects, the SEFL antibody comprises A287C, N297G, and L306C. In other exemplary aspects, the SEFL antibody comprises R292C, N297G, and V302C (i.e., SEFL2-2).

In certain embodiments, the anti-LILRB1 antibody, antigen binding antibody fragment thereof, competes for binding to LILRB1 with any antibody or antigen-binding fragment thereof described above. Exemplary antibody competition assay is described in detail in Example 6. In general, antigen (such as LILRB1) is typically bound by one reference antibody and probed by another. If the reference antibody prevents the binding of the probe antibody, then the antibodies are said to be competing with each other.

In certain embodiments, the antibody, or antigen-binding fragment thereof, binds the D3 domain of LILRB1, wherein said D3 domain comprises residues 222-312 of SEQ ID NO: 3 (or SEQ ID NO:6).

In certain embodiments, the antibody, or antigen-binding fragment thereof, binds the D4 domain of LILRB1, wherein said D4 domain comprises residues 313-409 of SEQ ID NO: 3 (or SEQ ID NO: 6).

Additional antibodies, or antigen-binding fragment thereof, of the invention can be obtained using LILRB1, LILRB2, or HLA-G (or antigenic fragment thereof) as an antigen. Exemplary sequences of human LILRB1, LILRB2, and HLA-G are provided herein as SEQ ID Nos. 3-18. Using these exemplary sequences as antigens, antibodies (or antigen-binding fragments thereof) of the invention can be obtained using art-known methods. For example, monoclonal antibodies may be made by the hybridoma method, or may be made by recombinant DNA methods. The antibodies may also be isolated from phage antibody libraries.

In certain embodiments, the antibody, or antigen-binding fragment thereof, of the invention binds to any one of SEQ ID NOs: 3-7, or an antigenic fragment of any one of SEQ ID NOs: 3-7. In certain embodiments, the antibody, or antigen-binding fragment thereof, of the invention binds to an amino acid sequence that is at least 70%, at least 75%, at least 80%, at least 85%, at least 90%, at least 95%, at least 97%, or at least 99% identical to any one of SEQ ID NOs: 3-7, or an antigenic fragment of any one of SEQ ID NOS: 3-7.

In certain embodiments, the antibody, or antigen-binding fragment thereof, of the invention binds to any one of SEQ ID NOs: 8-11, or an antigenic fragment of any one of SEQ ID NOS: 8-11. In certain embodiments, the antibody, or antigen-binding fragment thereof, of the invention binds to an amino acid sequence that is at least 70%, at least 75%, at least 80%, at least 85%, at least 90%, at least 95%, at least 97%, or at least 99% identical to any one of SEQ ID NOS: 8-11, or an antigenic fragment of any one of SEQ ID NOS: 8-11.

In certain embodiments, the antibody, or antigen-binding fragment thereof, of the invention binds to any one of SEQ ID NOs: 12-18, or an antigenic fragment of any one of SEQ ID NOS: 12-18. In certain embodiments, the antibody, or antigen-binding fragment thereof, of the invention binds to an amino acid sequence that is at least 70%, at least 75%, at least 80%, at least 85%, at least 90%, at least 95%, at least 97%, or at least 99% identical to any one of SEQ ID NOs: 12-18, or an antigenic fragment of any one of SEQ ID NOS: 12-18. The antibody, or antigen-binding fragment thereof, can bind to membrane-bound isoforms (e.g., HLA-G1, HLA-G2, HLA-G3, HLA-G4), soluble isoforms (e.g., HLA-G5, HLA-G6, HLA-G7), and soluble forms generated by proteolytic cleavage of membrane-bound isoforms (e.g. sHLA-G1).

Other LILRB1/2 pathway inhibitors that are useful for the invention include, for example, an immunoadhesin that specifically binds to LILRB1, LILRB2, or HLA-G, a fusion protein containing the extracellular portion of LILRB1, LILRB2, or HLA-G fused to a constant region (such as an Fc region of an immunoglobulin molecule).

Antisense RNA, or small interfering RNA (siRNA) may also be used as LILRB1/2 pathway inhibitors. Various art-known methods can be used to analyze the sequence of LILRB1, LILRB2, and HLA-G (such as SEQ ID Nos. 3-18) to obtain antisense RNA or siRNA that inhibits the expression of LILRB1, LILRB2, and/or HLA-G. siRNA design algorithms has been described above.

In certain embodiments, the LILRB1/2 pathway inhibitor is a short interfering RNA (siRNA) that reduces the expression level of HLA-G, as compared to a control. Exemplary siRNA that reduces the expression level of HLA-G can be found, e.g., in WO 2012/098234. In an exemplary embodiment, the siRNA comprises SEQ ID NO: 31.

A variety of controls may be used. HLA-G expression level from a non-cancer cell from the same patient, or from a cell from a healthy individual may be used as a control. Alternatively, the control may be expression level of HLA-G from the individual being treated at a time prior to treatment or at a time period earlier during the course of treatment. Still other controls may include a pre-determined expression level (e.g., expression data from public database, or from scientific literature).

In some embodiments, the LILRB1/2 pathway inhibitor described herein reduces the negative co-stimulatory signal mediated by or through cell surface proteins expressed on T lymphocytes mediated signaling through LILRB1/2, so to render a dysfunctional T-cell less dysfunctional (e.g., enhancing effector responses to antigen recognition).

### 3.3. Methods of Making Antibodies

Suitable methods of making antibodies and antigen-binding fragments thereof are known in the art. For instance, standard hybridoma methods for producing antibodies are described in, e.g., Harlow and Lane (eds.), Antibodies: A Laboratory Manual, CSH Press (1988), and CA. Janeway et al. (eds.), Immunobiology, 5th Ed., Garland Publishing, New York, NY (2001)). An exemplary method of preparing anti-PD-1 monoclonal antibodies or the present disclosure is provided herein in EXAMPLES.

Depending on the host species, various adjuvants can be used to increase the immunological response leading to greater antibody production by the host. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Other methods of antibody production are summarized in Table 1.

TABLE 1

| Technique | Exemplary references |
|---|---|
| EBV-hybridoma methods and | Haskard and Archer, J. Immunol. Methods, 74(2), 361- |
| Bacteriophage vector expression | 67 (1984), Roder et al., Methods Enzymol., 121, 140-67 |
| systems | (1986), and Huse et al., Science, 246, 1275-81 (1989)). |
| methods of producing antibodies in non- | U.S. Pat. Nos. 5,545,806, 5,569,825, and 5,714,352, and |
| human animals | U.S. patent application Publication No. 2002/0197266 |
| inducing in vivo production in the | Orlandi et al (Proc Natl Acad Sci 86: 3833-3837; 1989), |
| lymphocyte population or by screening | and Winter G and Milstein C (Nature 349: 293-299, |
| recombinant immunoglobulin libraries or | 1991). |
| panels of highly specific binding | |
| reagents | |
| methods of producing recombinant | Protein production and purification″ Nat Methods 5(2): |
| proteins | 135-146 (2008). |
| Phage display | Janeway et al., supra, Huse et al., supra, and |
| | U.S. Pat. No. 6,265,150). Related methods also are |
| | described in U.S. Pat. No. 5,403,484; U.S. Pat. No. |
| | 5,571,698; U.S. Pat. No. 5,837,500; U.S. Pat. No. |
| | 5,702,892. The techniques described in U.S. Pat. No. |
| | 5,780,279; U.S. Pat. No. 5,821,047; U.S. Pat. No. |
| | 5,824,520; U.S. Pat. No. 5,855,885; U.S. Pat. No. |
| | 5,858,657; U.S. Pat. No. 5,871,907; U.S. Pat. No. |
| | 5,969,108; U.S. Pat. No. 6,057,098; and U.S. Pat. |
| | No. 6,225,447 |
| Antibodies can be produced by | U.S. Pat. Nos. 5,545,806 and 5,569,825, and |
| transgenic mice | Janeway et al., supra. |

Methods of testing antibodies, or antigen-binding fragments thereof, for the ability to bind to intended target (e.g., PD-1, PD-L1, LILRB1, LILRB2, or HLA-G) regardless of how the antibodies are produced are known in the art and include any antibody-antigen binding assay, such as, for example, radioimmunoassay (RIA), ELISA, Western blot, immunoprecipitation, SPR, and competitive inhibition assays (see, e.g., Janeway et al., infra, and U.S. Patent Application Publication No. 2002/0197266, and the above section relating to competition assays). Other binding assays, e.g., competitive binding assays or competition assays, which test the ability of an antibody to compete with a second antibody for binding to an antigen, or to an epitope thereof, are known in the art and can be used to test the ability of an antibody to bind to PD-1, PD-L1, LILRB1, LILRB2, or HLA-G. See, e.g., U.S. Patent Application Publication No. US20140178905, Chand et al., Biologicals 46:168-171 (2017); Liu et al., Anal Biochem 525:89-91 (2017); and Goolia et al., J Vet Diagn Invest 29 (2): 250-253 (2017). Also, other methods of comparing two antibodies are known in the art, and include, for example, surface plasmon resonance (SPR). SPR can be used to determine the binding constants of the antibody and second antibody and the two binding constants can be compared.

In certain embodiments, the affinity of the antibody, or antigen-binding fragment thereof, to an intended antigen may be described in terms of K_{D}. In exemplary aspects, the Ko of the antibody provided herein is about 10⁻¹ M or less, about 10⁻² M or less, about 10⁻³ M or less, about 10⁻⁴ M or less, about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less, from about 10⁻⁵ M to about 10⁻¹⁵ M, from about 10⁻⁶ M to about 10⁻¹⁵ M, from about 10⁻⁷ M to about 10⁻¹⁵ M, from about 10⁻⁸ M to about 10⁻¹⁵ M, from about 10⁻⁹ M to about 10⁻¹⁵ M, from about 10⁻¹⁰ M to about 10⁻¹⁵ M, from about 10⁻⁵ M to about 10⁻¹⁴ M, from about 10⁻⁶ M to about 10⁻¹⁴ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10-14 M, from about 10⁻⁵ M to about 10⁻¹³ M, from about 10⁻⁶ M to about 10⁻¹³ M, from about 10⁻⁷ M to about 10⁻¹³ M, from about 10⁻⁸ M to about 10⁻¹³ M, from about 10⁻⁹ M to about 10⁻¹³ M, or from about 10⁻¹⁰ M to about 10⁻¹³ M.

In certain embodiments, the K_{D} of the antibody, or antigen-binding fragment thereof, provided herein is micromolar, nanomolar, picomolar or femtomolar. In exemplary aspects, the K_{D} of the antibody provided herein is within a range of about 10⁻⁴ to 10⁻⁶ M, or 10⁻⁷ to 10⁻⁹ M, or 10⁻¹⁰ to 10⁻¹² M, 10⁻¹³ to 10⁻¹⁵ M. In exemplary embodiments, the antibody, or antigen-binding fragment thereof, has a K_{D} of less than 100 pM, optionally, about 1 pM to about 50 pM, such as about 1 pM to about 20 pM, or less than about 10 pM.

The value of Ko can be determined directly by well-known methods, and can be computed even for complex mixtures by methods such as those, for example, set forth in Caceci et al. (1984, Byte 9:340-362). For example, the K_{D} may be established using a double-filter nitrocellulose filter binding assay such as that disclosed by Wong & Lohman (1993, Proc. Natl. Acad. Sci. USA 90:5428-5432). Other standard assays to evaluate the binding ability of ligands such as antibodies towards target antigens are known in the art, including for example, ELISAs, Western blots, RIAs, and flow cytometry analysis, and other assays exemplified elsewhere herein.

One exemplary method for measuring binding affinity (K_{D}) value is surface plasmon resonance (SPR), typically using a biosensor system such as a BIACORE® system. SPR refers to an optical phenomenon that allows for the analysis of real-time biospecific interactions by detection of alterations in protein concentrations within a biosensor matrix, for example using the BIACORE® system. BIAcore kinetic analysis comprises analyzing the binding and dissociation of an antigen from a chip with an immobilized molecule (e.g., a molecule comprising an antigen-binding domain), on their surface; or the dissociation of an antibody, or antigen-binding fragment thereof, from a chip with an immobilized antigen.

In certain embodiments, the affinity of an antibody is measured according to the conditions provided in Example 6.

### 3.4. Methods of Treatment

The combination therapy described herein can be used to increase IFN-γ expression level in a subject that has cancer, and/or to increase CD8+ T-cell mediated cytotoxicity in a subject that has cancer.

It has been reported that IFN-γ related mRNA profile can predict clinical response to PD-1 blockade (Ayers et al., J Clin Invest. 2017; 127 (8): 2930-2940). Host immune system is capable of recognizing and eliminating dysplastic and neoplastic cells. Certain factors, such as exposure to ultraviolet radiation, smoking, and chronic viral infection, can damage critical genes involved in DNA replication and repair, resulting in abnormal cellular growth. In response, an orchestrated innate and adaptive antitumor immune response is initiated that may lead to the production of IFN-γ. IFN-γ is a key cytokine produced by activated T cells, as well as natural killer (NK) and NK T cells, in the tumor microenvironment, and it plays an important role in coordinating this process. However, the same IFN-γ signaling processes can ultimately induce feedback inhibition that compromises antitumor immunity. As part of this feedback loop, IFN-γ signaling enables the PD-1 signaling axis to become activated through direct upregulation of the ligands PD-L1 and PD-L2 in tumor, immune infiltrate, and stromal cells, which interact with PD-1 on tumor-infiltrating T cells to downregulate the cytotoxic response. In addition, IFN-γ can upregulate expression of other key immune suppressive molecules such as IDO1 within the tumor microenvironment. Tumor adaptation takes advantage of this delicate balance of positive and negative immune signaling factors, allowing the cancer to survive and progress.

Studies have also shown that it IFN-γ can enhance anti-tumor and antiviral effects of CD8+ T cells. The CD8+ T cells are able to produce IFNγ, which enhances their ability to migrate to the site of antigen-presenting cells. Conversely, deprivation of either autocrine or paracrine IFNγ, or blockade of IFNγ signaling to CTL markedly reduced their cytotoxic function, their kinematics, and effector cell survival. The need for local IFNγ to enable cytotoxic CD8+ T-cell function is significant for cancer treatment.

The treatment method described herein can be used to treat a variety of cancers, especially cancer patients who are poor responders to the treatment using an PD-1/PD-L1 pathway inhibitor. As shown in the examples, the combination is particularly effective in patients who responded to anti-PD-1 treatment poorly. For certain patients (see, e.g., FIG. 7, Pair #1), mono-therapy using either anti-PD-1, or anti-LILRB1 failed to improve CD8+ T cell mediated cytotoxicity as compared to control. However, when anti-PD-1 and anti-LILRB1 are combined, the increase in CD8+ T cell cytotoxicity was more than doubled.

Accordingly, provided herein are methods of increasing T cell activity, in particular CD8+ T-cell mediated cytotoxicity in a subject. Such increase in T cell activity includes, e.g., increasing T cell survival and effector function, restricting terminal differentiation and loss of replicative potential, promoting T cell longevity, and enhancing cytotoxicity against target (e.g., cancer) cells. In certain embodiments, the T cell activity or immune response is directed against a cancer cell or cancer tissue or a tumor cell or tumor. In certain embodiments, the immune response is a humoral immune response. In certain embodiments, the immune response is an innate immune response. In certain embodiments, the immune response which is enhanced is a T-cell mediated immune response.

In certain embodiments, the cancer is a solid tumor. In some embodiments, the cancer is brain cancer, bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), Squamous Cell Carcinoma of the Head and Neck (SCCHN), chronic myelogenous leukemia (CML), small lymphocytic lymphoma (SLL), malignant mesothelioma, colorectal cancer, or gastric cancer.

Additional cancers can be treated include acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, and bronchioloalveolar carcinoma.

In certain embodiments, the cancer is non-small cell lung cancer (NSCLC), head and neck cancer, renal cancer, triple negative breast cancer, or gastric cancer. In certain embodiments, the cancer is non-small cell lung cancer (NSCLC), small cell lung cancer (SCLC), head and neck cancer, renal cancer, breast cancer, melanoma, ovarian cancer, liver cancer, pancreatic cancer, colon cancer, prostate cancer, gastric cancer, lymphoma or leukemia.

In certain embodiments, the cancer is brain cancer.

In certain embodiments, the cancer is brain cancer, bladder cancer, breast cancer, clear cell kidney cancer, cervical cancer, colon cancer, rectal cancer, endometrial cancer, gastric cancer, head/neck squamous cell carcinoma, lip cancer, oral cancer, liver cancer, lung squamous cell carcinoma, melanoma, mesothelioma, non-small-cell lung cancer (NSCLC), non-melanoma skin cancer, ovarian cancer, pancreatic cancer, prostate cancer, renal cell carcinoma, sarcoma, small-cell lung cancer (SCLC), Squamous Cell Carcinoma of the Head and Neck (SCCHN), triple negative breast cancer, or thyroid cancer.

In certain embodiments, the cancer is adrenocortical tumor, alveolar soft part sarcoma, carcinoma, chondrosarcoma, colorectal carcinoma, desmoid tumors, desmoplastic small round cell tumor, endocrine tumors, endodermal sinus tumor, epithelioid hemangioendothelioma, Ewing sarcoma, germ cell tumor, hepatoblastoma, hepatocellular carcinoma, melanoma, nephroma, neuroblastoma, non-rhabdomyosarcoma soft tissue sarcoma (NRSTS), osteosarcoma, paraspinal sarcoma, renal cell carcinoma, retinoblastoma, rhabdomyosarcoma, synovial sarcoma, or Wilms tumor.

In certain embodiments, the cancer is acute lymphoblastic leukemia (ALL), acute myeloid leukemia (AML), chronic lymphocytic leukemia (CLL), or chronic myeloid leukemia (CML).

In certain embodiments, the cancer is diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), or small lymphocytic lymphoma (SLL).

In certain embodiments, the cancer tests positive for the expression of PD-L1, PD-L2, or combination thereof. In certain embodiments, the cancer has increased PD-L1 and/or PD-L2 expression, as compared to a normal cell. In certain embodiments, the individual is a human and the cancer is a solid tumor that tests positive for human PD-L1 and/or PD-L2.

In certain embodiments, the cancer tests positive for the expression of HLA-G. In certain embodiments, the cancer has increased HLA-G expression, as compared to a normal cell. In certain embodiments, the individual is a human and the cancer is a solid tumor that tests positive for human HLA-G.

CD8+ T-cell mediated cytotoxicity, as well as the likely outcome of treatment, can be assessed by art-known methods (e.g., by measuring IFN-γ levels in the subject). Measurement of IFN-γ levels in a subject can be carried out in any suitable tissue or bodily fluid sample from the subject. The measured CD8+ T-cell mediated cytotoxicity (such as level of IFN-γ) can then be compared to a control or a reference. The control can be CD8+ T-cell mediated cytotoxicity level (such as level of IFN-γ) from a non-cancer cell from the same patient, or from a cell from a healthy individual. Alternatively, the control may be the level of CD8+ T-cell mediated cytotoxicity (such as IFN-γ expression level) from the individual being treated at a time prior to treatment or at a time period earlier during the course of treatment. Still other controls may include a pre-determined level (e.g., T cell cytotoxicity data such as IFN-γ expression data from public database, or from scientific literature). The control can be corrected for demographic variation and the like, if necessary, using methods that are well known in the art.

The levels of IFN-γ expression, may be measured at the protein or nucleic acid level. For example, measurement of protein expression at the protein level can be performed by immunoassays such as ELISA (direct and sandwich), immunohistochemistry, Western blot, radioimmunoassay, flow cytometry and neutralization, although the skilled artisan will be aware of other methods that are well known and available. Suitable immunoassay methods are described, for example, in U.S. Pat. No. 4,666,865. Suitable reagents and kits to perform these tests are available from R&D Systems Inc., Minnesota. Suitable reagents and kits available from R&D Systems Inc., to measure human IFN-γ include anti-human IFN-γ antibody (Cat. No. AF-285-NA); biotinylated anti-human IFN-γ antibody (Cat. No. BAF285); DuoSet® ELISA for human IFN-γ (Cat. No. DY285); human IFN-γ development module (Cat. No. SEL285); ELISpot for human IFN-γ (Cat. No. EL285); Carboxyfluorescein conjugated IFN-γ monoclonal antibody (Cat. No. IC285F); Fluorokine MAP human IFN-γ kit (Cat. No. LU285); Monoclonal anti-human IFN-γ antibody Cat. No. MAB285); Phycoerythrin-conjugated monoclonal IFN-γ antibody (Cat. No. IC285P); Quantkine human IFN-γ ELISA kit (Cat. No. DIF50); anti-human IFN-γ polyclonal antibody (Cat. No. AB 250 NA) and affinity purified anti-human IFN-γ polyclonal antibody (Cat. No. AF 250 NA). A radioimmunoassay kit for human IFN-γ is available from Celltech, Berkshire, England. Alternatively, mass spectrometry, chromatography (including high pressure liquid chromatography), gel electrophoresis and biological activity assays can be used to measure IFN-γ levels and activity in human samples.

Measurement of protein expression at the nucleic acid level can be achieved by quantitative RT-PCR methods, PCR-ELISA, and in situ hybridization techniques that are well known the art. Suitable reagents and kits available from R&D Systems Inc., to measure human IFN-γ mRNA expression include human IFN-gamma Quantikine mRNA kit (Cat. No. KRNIF0-0); human IFN-gamma primer pair (Cat. No. RDP-14-025); Quantikine mRNA Probes and Calibrator Kit for human IFN-γ (Cat. No. RNIF0-036). Alternatively, microarray-based methods and the like may be used. Such microarray methods are commercially available, for example from Affymetrix (Sunnyvale, Calif.).

The biological sample can be a body fluid, such as plasma, serum, urine, cerebral spinal fluid, and sputum. The biological sample can also be tissue samples, such as biopsy.

Additional methods of measuring T cell activity and immune responses are known in the art. T cell activity can be measured by, for example, a cytotoxicity assay, such as those described in Fu et al., PLOS ONE 5 (7): e11867 (2010). Other T cell activity assays are described in Bercovici et al., Clin Diagn Lab Immunol. 7 (6): 859-864 (2000). Methods of measuring immune responses are described in e.g., Macatangay et al., Clin Vaccine Immunol 17 (9): 1452-1459 (2010), and Clay et al., Clin Cancer Res.7 (5): 1127-35 (2001).

### 4. Pharmaceutical Compositions and Administration

In another aspect, the invention also provides a pharmaceutical composition comprising the PD-1 pathway inhibitor and LILRB1/2 pathway inhibitor described herein.

The PD-1 pathway inhibitor and LILRB1/2 pathway inhibitor described herein can be prepared as two separate compositions. The two compositions can be administered separately, or be mixed together at bedside right before administration.

Alternatively, the PD-1 pathway inhibitor and LILRB1/2 pathway inhibitor described herein can be prepared as a single, pre-mixed composition.

In other embodiments, the two inhibitors can be conjugated into a single molecule, then be formulated into a single composition. For example, the composition can comprise a bi-specific antibody, with one arm binds PD-1 or PD-L1, and the other arm binds LILRB1, LILRB2, or HLA-G.

The pharmaceutical composition may additionally comprise a pharmaceutically acceptable carrier, diluents, or excipient. Standard pharmaceutical carriers include a phosphate buffered saline solution, water, emulsions such as an oil/water or water/oil emulsion, and various types of wetting agents.

The pharmaceutical composition can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

The pharmaceutical compositions can be formulated to achieve a physiologically compatible pH. In some embodiments, the pH of the pharmaceutical composition can be for example between about 4 or about 5 and about 8.0 or about 4.5 and about 7.5 or about 5.0 to about 7.5. In exemplary embodiments, the pH of the pharmaceutical composition is between 5.5 and 7.5.

The PD-1 pathway inhibitor and LILRB1/2 pathway inhibitor described herein can be administered to the subject via any suitable route of administration, such as parenteral, nasal, oral, pulmonary, topical, vaginal, or rectal administration. Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. For additional details, see Pharmaceutics and Pharmacy Practice, J. B. Lippincott Company, Philadelphia, PA, Banker and Chalmers, eds., pages 238-250 (1982), and ASHP Handbook on Injectable Drugs, Toissel, 4th ed., pages 622-630 (1986)).

The dose of the active agent of the present disclosure should be sufficient to treat cancer as described herein in a period of from about 1 to 4 minutes, 1 to 4 hours or 1 to 4 weeks or longer, e.g., 5 to 20 or more weeks, from the time of administration. In certain embodiments, the time period could be even longer. The dose will be determined by the efficacy of the particular active agent and the condition of the animal (e.g., human), as well as the body weight of the animal (e.g., human) to be treated.

Many assays for determining an administered dose are known in the art. For purposes herein, an assay, which comprises comparing the extent to which cancer is treated upon administration of a given dose of the active agent of the present disclosure to a mammal among a set of mammals, each set of which is given a different dose of the active agent, could be used to determine a starting dose to be administered to a mammal. The extent to which cancer is treated upon administration of a certain dose can be represented by, for example, the cytotoxicity of the active agent or the extent of tumor regression achieved with the active agent in a mouse xenograft model. Methods of measuring cytotoxicity of the fusion proteins and methods of assaying tumor regression are known in the art.

The dose of the active agent of the present disclosure also will be determined by the existence, nature and extent of any adverse side effects that might accompany the administration of a particular active agent of the present disclosure. Typically, the attending physician will decide the dosage of the active agent of the present disclosure with which to treat each individual patient, taking into consideration a variety of factors, such as age, body weight, general health, diet, sex, active agent of the present disclosure to be administered, route of administration, and the severity of the condition being treated. By way of example and not intending to limit the present disclosure, the dose of the active agent of the present disclosure can be about 0.0001 to about 1 g/kg body weight of the subject being treated/day, from about 0.0001 to about 0.001 g/kg body weight/day, or about 0.01 mg to about 1 g/kg body weight/day. Dosage units may be also expressed in rag/m², which refer to the quantity in milligrams per square meter of body surface area.

Each therapeutic agent in the combination therapy described herein may be administered simultaneously (e.g., in the same medicament or at the same time), concurrently (i.e., in separate medicaments administered one right after the other in any order or sequentially in any order. Sequential administration may be useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In certain embodiments, the PD-1 pathway inhibitor and the LILRB1/2 pathway inhibitor are combined or co-formulated in a single dosage form. In certain embodiments, the PD-1 pathway inhibitor and the LILRB1/2 pathway inhibitor are administered separately. Although the simultaneous administration of the PD-1 pathway inhibitor and the LILRB1/2 pathway inhibitor may be maintained throughout a period of treatment, anti-cancer activity may also be achieved by subsequent administration of one compound in isolation (for example, the PD-1 pathway inhibitor without the LILRB1/2 pathway inhibitor following initial combination treatment, or alternatively, the LILRB1/2 pathway inhibitor without the PD-1 pathway inhibitor following initial combination treatment). In some embodiments, the PD-1 pathway inhibitor is administered before administration of the LILRB1/1 pathway inhibitor, while in other embodiments, the PD-1 pathway inhibitor is administered after administration of the LILRB1/2 pathway inhibitor. In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

The combination therapy of the invention may be used prior to or following surgery to remove a tumor and may be used prior to, during or after radiation therapy. The combination therapy of the invention may be used to treat a tumor that is large enough to be found by palpation or by imaging techniques well known in the art, such as MRI, ultrasound, or CAT scan. In some embodiments, the combination therapy of the invention is used to treat an advanced stage tumor having dimensions of at least about 200 mm³, 300 mm³, 400 mm³, 500 mm³, 750 mm³, or up to 1000 mm³.

### EXAMPLES

### Introduction

T cells, especially antigen specific cytotoxic T cells, can detect and eliminate cancer cells through the recognition of tumor associated antigens such as neo-antigens. Neoplastic cells, however, evade the immune recognition through various mechanisms. For example, tumor infiltrating T cells often fail to eliminate cancer due to an immunosuppressive tumor microenvironment that promotes a dysfunctional T cell state, often termed “exhaustion”, characterized by the expression of multiple inhibitory receptors, such as PD-1, TIM3, and CTLA-4. Strategies aimed at boosting immune cell recognition of cancer and T cell cytotoxicity against tumor cells have long been used to treat cancers. For example, cytokines such as IFN-α and IL-2, which promote antigen presentation and T cell activity, respectively, have been used to treat melanoma and other cancers for many years. Their anti-tumor effects, however, have been limited by severe, dose-limiting side effects. In recent years, checkpoint inhibitors (including antibodies targeting CTLA-4 and PD-1 that are expressed on exhausted T cells and regulatory T cells) have been able to enhance the anti-tumor effector functions of T cells and have shown dramatic therapeutic benefit in various cancer types, although only in a subset of patients.

In human peripheral blood, there are several CD8+ T cell subsets, in addition to naïve and memory CD8+ cells, CD45RA and CCR7 markers also define T_{EMRA} subset (CD45RA+ CCR7−). These cells are antigen-experienced effector cells with high cytotoxicity, high sensitivity to apoptosis, low IL-2 production, and low proliferative capability. In addition, these cells represent a significant CD8+ T cell population in elder individual and cancer patients. Several other markers such as CD27, CD28, and CD57 are also used to define the heterogeneity of T_{EMRA} cells. T_{EMRA} cells are found to express many NK-like receptors, such as KLRG-1 and LILRB1.

Exhausted CD8+ T cells were reported to dominantly infiltrate various solid tumors. These cells highly express various inhibitory co-receptors such as PD-1, TIM3, and LAG3. However, it is unclear whether T_{EMRA} cells are also present in solid tumors, and whether PD-1 pathway plays a role in regulating their activities.

LILRB1 belongs to leukocyte Ig-like receptor subfamily B that is expressed in various types of immune cells to suppress their activation. LILRB1 is the only member in LILR family that is expressed on subsets of CD8+ T cells, as well as some NK cells and B cells. It contains 4 lg-like domains extracellularly that mediates its interaction with ligands (i.e., non-classical class I molecule HLA-G). LILRB1 has 4 ITIM motifs in its intracellular domain that recruit SHP1 and SHP2 upon ligation to inhibit ITAM signaling.

In this study, we demonstrated that LILRB1 was a good marker to identify effector CD8+ cells and it exerted a negative regulatory function during BiTER molecule-induced tumor killing. More importantly, we discovered that CD8+ subset expressing of LILRB1 and CD8+ subset expressing PD-1 are non-overlapping—each marker is uniquely associated with different CD8+ T cell subsets. Blocking both pathways could synergistically enhance CD8+ T cell function.

### Example 1. LILRB1 Inhibits CD8+ Effector T Cell Activation and Function in A TDCC Assay

In order to understand which human CD8+ subset most efficiently mediated BiTER molecule-induced TDCC, we isolated human CD8+ naive (T_{N}), effector memory (TEM), and effector or effector memory RA (T_{EMRA}) subsets based on expression of surface markers CD45RA and CCR7 from peripheral blood. The cytolytic activity of isolated CD8+ T cell subsets was determined by utilizing a MART-1 BiTE® molecule-mediated SK-MEL-2 tumor cell killing assay. We discovered that CD45RA+ CCR7-T_{EMRA} cells were the most effective subset in killing the target cancer cells induced by BiTE® molecule (EC50=25.51 pM), followed by CD45RA CCR7-TEM cells (EC50=58.11 pM) and CD45RA CCR7-T_{N} cells (EC50=7948 nM). T_{EMRA} cells also had the highest maximum percentage of tumor cell lysis in the assay.

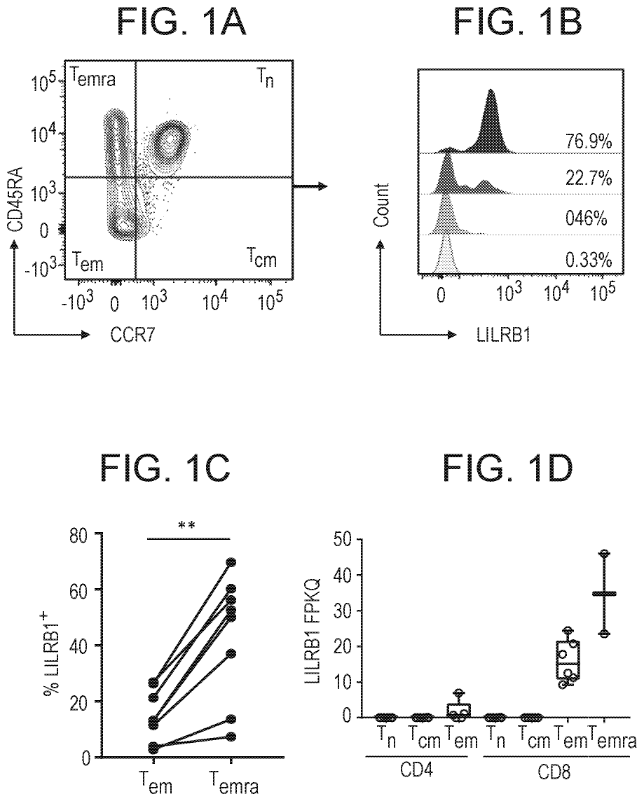

These data suggested that enhancing the effector function of T_{EMRA} could further enhance BiTER molecule-mediated tumor killing. To identify regulatory pathways that modulating CTL activity of these CD8+ subsets, we compared their gene transcriptional profiles. As expected, memory CD8+ T cells highly expressed various genes, including PRF1, GZMB, and CX3CR1, which were associated CD8+ T cell effector function. In addition, we also identified the co-inhibitory molecule LILRB1 as being preferentially expressed in T_{EMRA} and TEM CD8+ T cell subsets (FIG. 1D). FACS analysis confirmed majority of T_{EMRA} cells highly expressed LILRB1, and a minor TEM CD8+ subsets also had detectable level of LILRB1 expression (FIGS. 1A-1C).

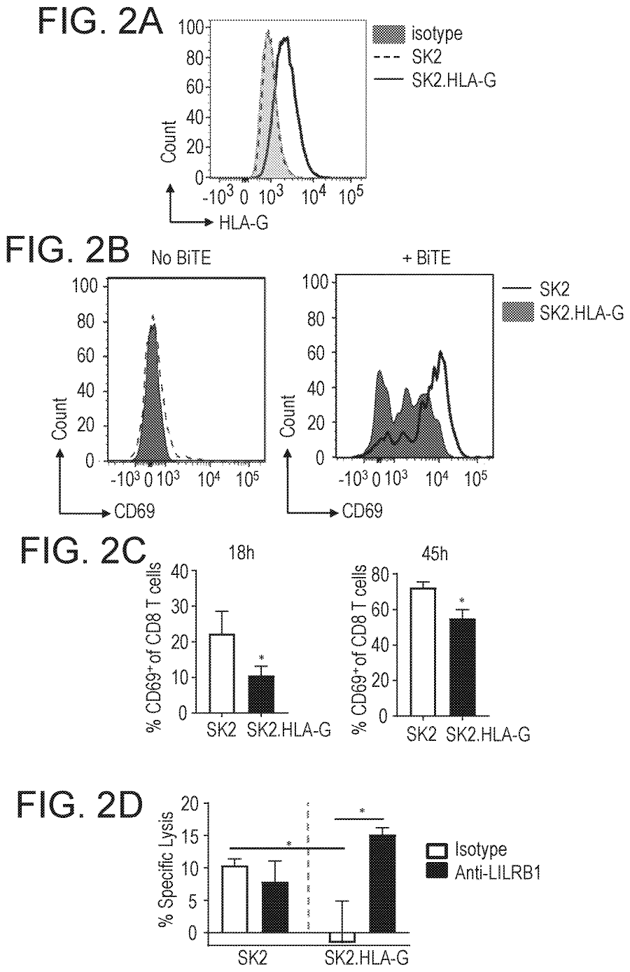

In order to elucidate if LILRB1 regulates the function of CD8+ effector cells in our BiTE® molecule system, we performed CD8+ T cell killing assays utilizing a MART-1 BiTE® molecule. We engineered SK-MEL-2 target cells to express HLA-G, the ligand for LILRB1 (FIG. 2A). T_{EMRA} CD8+ T cells were enriched from healthy donor PBMC by flow sorting and were co-cultured with parental SK-MEL-2 target cells (SK2) or HLA-G-overexpressing SK-MEL-2 cells (SK2.HLA-G) in the presence of anti-LILRB1 blocking or control antibodies. Effector CD8+ function was stimulated by a suboptimal concentration of MART-1 BiTE® molecule, and measured as specific target cell lysis. Our data showed that HLA-G-expression on target cells dramatically reduced BiTE® molecule-induced CD8+ T cell cytolytic activity against tumor cells (FIG. 2D). LILRB1 blockade significantly enhanced CTL function to kill HLA-G-expressing tumor cells in this assay (FIG. 2D). Presence of HLA-G on tumor cells also significantly inhibited BiTER molecule-mediated upregulation of CD69 on effector CD8+ T cells (FIGS. 2B and 2C). In summary, these data support the conclusion that LILRB1 functions as a negative regulator of CD8+ effector T cell function and that blocking LILRB1 can enhance BiTER molecule-mediated tumor lysis by effector CD8+ cells.

### Materials and Methods

Primary human T cell isolation. Human PBMCs from healthy volunteers were obtained after informed consent and authorized by Amgen Research Blood Donor Program. Peripheral blood mononuclear cells (PBMCs) were isolated by density gravity centrifugation (Ficoll-Paque™ PREMIUM; GE Healthcare). Isolated cells were incubated with monoclonal antibodies (CD3-APC (Sk7, eBioscience), CD4-BV605 (OKT4, Biolegend), CD8-AF700 (RPA-T8, Biolegend), CCR7-BV421 (G043H7, Biolegend), CD45RA-APC-Cy7 (HI100, Biolegend)) for 30 min at 4° C. with 7AAD for live dead staining (BD Biosciences) followed by thorough washing. Cell subsets were then isolated from stained PBMC by cell sorting with BD FACSAria™ cytometer based on surface marker expression, such that CD45RA+ CCR7+ cells as T_{N}, CD45RA CCR7+ cells as TCM, CD45RA CCR7 cells as TEM, and CD45RA+ CCR7-cells as T_{EMRA}, in CD4+ (gated as CD3+CD4+CD8) and CD8+ (gated as CD3+CD4 CD8+) cells.

Flow cytometry. Isolated cells were stained with indicated monoclonal antibodies (CD3-APC (Sk7, eBioscience), CD4-BV605 (OKT4, Biolegend), CD8-AF700 (RPA-T8, Biolegend), CCR7-BV421 (G043H7, Biolegend), CD45RA-APC-Cy7 (HI100, Biolegend), LILRB1-PE (GHI/75, Biolegend), and PD-1-BV650 (EH12.1, BD Bioscience), with 7AAD for live dead staining (BD Biosciences). Cells were incubated with appropriate antibodies for 30 min at 4° C. followed by washing with FACS buffer (BD Biosciences). Sample processing was performed on a LSR II (BD Biosciences) and with FlowJo™ 10 software (Tree Star).

RNA Sequencing. Total RNAs were isolated from sorted T cell subsets (Qiagen). PolyA tailed mRNAs were isolated with Oligo-dT beads and RNA library was prepared following standard protocol.

Target Cell lines. Parental SK-MEL-2 (SK2) were maintained EMEM medium (ATCC) with 10% FBS and 1% penicillin/streptomycin at 37° C. Cells with stable expression of HLA-G were established by retroviral transduction. The HLA-G. B2 microglobulin cDNA was inserted into retrovirus vector pLHCX and was co-transfected with Lipofectamine 3000 (Thermo Fisher Scientific) into GP2-293 packaging cells with pVSV-G. Supernatant were collected 48 hours after transfection and filtered. Then, SK2 cells were transduced with a mixture of viral supernatant with polybrene at 5 μg/ml (EMD Millipore). After spin transduction for 1.5 h at 1200 g, 32° C., fresh media was added. HLA-G expression was confirmed by flow cytometry using LSR II (BD Biosciences).

Cytotoxicity assay. Isolated CD8+T subsets (T_{N}, TEM, and T_{EMRA}) as effector cells were co-cultured with target HLA-G expressing SK2 or SK2 tumor lines in the presence of different concentrations of the MART1 BiTE® molecule (Amgen) in black 96-well flat bottom plates with transparent bottoms (Nunc). The effector cell to target cell ratio was 4:1 (Lₑₓₚ). Co-culture of effector cells and target cells without BiTE® molecule as control (Lₘₐₓ). For blocking assay, 20 μg/ml of anti-LILRB1 (GHI/75, Biolegend) or isotype control antibody (Biolegend) were added in the assay. Cells were cultured in RPMI-1640 supplemented with 10% heat-inactivated FBS, 100 U/mL penicillin and streptomycin, 2-Mercaptoethanol (all from Gibco) at 37° C. After 45 h, T cells were removed by washing with warm media and stained for CD69 (FN50, Biolegend) followed by flow cytometry using LSR II (BD Biosciences). Samples were analyzed with FlowJo 10 software (Tree Star). Target cell were washed and cell viability was determined by CellTiter-Glo® (Promega). Luminescence was measured with EnVision® 2104 multilable reader (PerkinElmer). Percentage of specific target cell lysis was calculated by the formula: Specific lysis [%]=(1−Lₑₓₚ/Lₘₐₓ)×100.

### Example 2. Differential Expression of LILRB1 and PD-1 on Human CD8+ T Cells

PD-1 exerts essential repressive functions on CD8+ T cells, especially on tumor infiltrating exhausted CD8+ T cells. Therapeutic antibodies blocking PD-1 pathway can significantly enhance the effector functions and provide therapeutic benefits in many cancers. Given the fact that LILRB1 was also a negative regulator of CD8+ T cells, we studied the differences on the regulation and expression of LILRB1 and PD-1 on CD8+ T cell subsets.

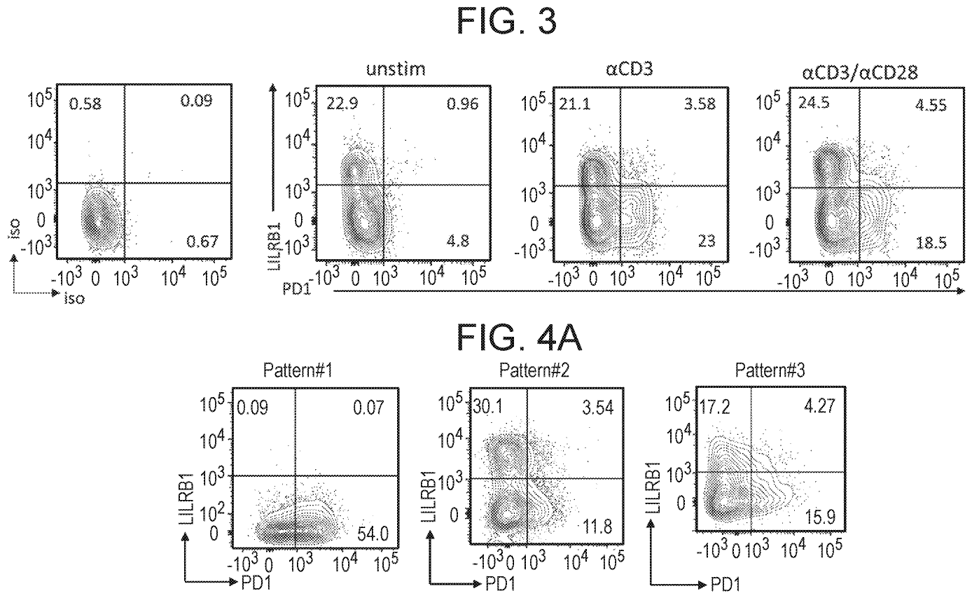

Human PBMCs from healthy donor were stimulated with plate-bound anti-CD3 or plate-bound anti-CD3 plus anti-CD28 for 48 hours. Activated cells were collected for FACS analysis. Ex vivo FACS was performed with unstimulated PBMCs as control. FIG. 3 provides representative FACS plots showing LILRB1 and PD-1 expression on gated CD8+ T cells under the indicated conditions. Our data demonstrate that LILRB1 and PD-1 are differentially expressed by human CD8+ T cells following activation, with PD-1 being upregulated predominantly in LILRB1 negative populations.

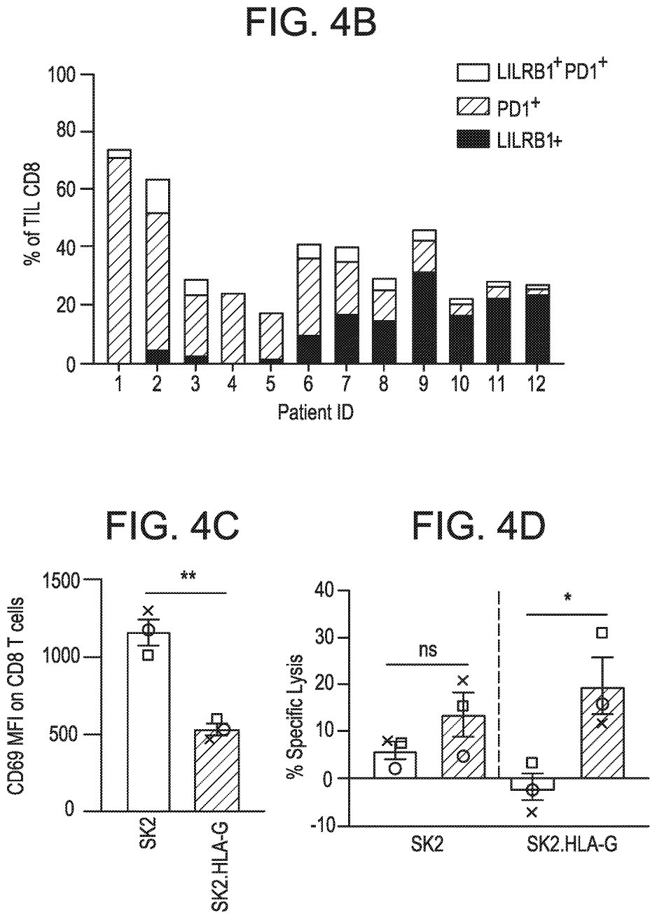

We further discovered that PD-1 was highly expressed in TEM (effector memory cell, CD45RA-CCR7−) cells, while LILRB1 was highly expressed in TEFF (effector T cell, CD45RA+ CCR7−) cells. In addition, LILRB1 is highly expressed in T_{EMRA} CD8+ T cells, cells with most potent effector function. Representative data are shown in FIG. 1. The discovery is surprising, as most immune checkpoint proteins we studied are co-expressed with PD-1. LILRB1 is an exception. In fact, our data suggest that the expression of LILRB1 and PD-1 is likely mutually exclusive in tumor infiltrating CD8+ T cells (see e.g., FIGS. 5A-5B and FIGS. 4A-4B).

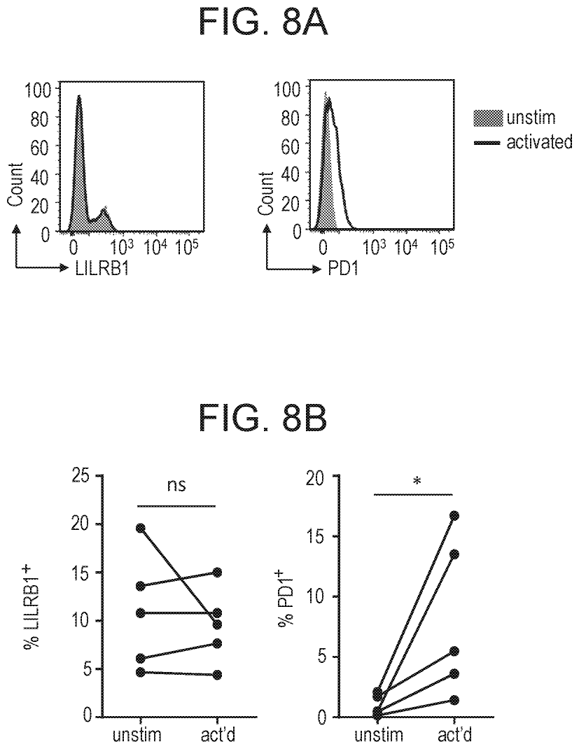

The expression of LILRB1 and PD-1 on CD8+ T cells is also regulated differently. We discovered that while PD-1 expression is regulated by TCR activation (FIGS. 8A-8B). LILRB1 expression was not sensitive to TCR signaling (FIGS. 8A-8B). Blocking PD-1 pathway can further promote effector function of CD8+ T cells. Consistently, LILRB1+ effector cell population was significantly increased in 2 day activated CD8+ TEM cells when PD-1 pathway was blocked by recombinant PD-L1, while the expression of PD-1 was not significantly altered (FIGS. 8C-8D). Furthermore, effector promoting cytokines such as IL-2 and IL-15, but not TNFα, also significantly increased the LILRB1 expression on T cells (data not shown). Thus, strategies that enhanced CD8+ effector function, but not TCR activation alone, increased LILRB1+CD8+ effector population.

### Materials and Methods

Isolated human PBMCs were stimulated with 10 ng/ml recombinant human IL2, 10 ng/ml recombinant human IL15, or 100 ng/ml recombinant human TNF (all from R&D Systems) for 24 h. Sorted human CD8+ TEM cells were activated with plate-bound anti-CD3 (OKT3, 5 g/ml, BD Biosciences) plus soluble anti-CD28 (CD28.2, 2 μg/ml, BD Biosciences) for 48 h. When indicated, 10 μg/ml recombinant human PD-L1 (Biolegend) or same amount of human IgG1 isotype control antibody were added in culture medium. Cells were cultured at 37° C. in RPMI-1640 supplemented with 10% heat-inactivated FBS, 100 U/mL penicillin and streptomycin, 2-Mercaptoethanol (all from Gibco)).

For flow cytometry, cells were stained with monoclonal antibodies (CD3-APC (Sk7, eBioscience), CD4-BV605 (OKT4, Biolegend), CD8-AF700 (RPA-T8, Biolegend), LILRB1-PE (GHI/75, Biolegend), and PD-1-BV650 (EH12.1, BD Bioscience). 7AAD was used for live dead staining (BD Biosciences). Cells were incubated with antibodies for 30 min at 4° C. followed by washing with FACS buffer (BD iosciences). Samples were acquired with LSR-II analyzer and analyzed using FlowJo software (v10.3).

### Example 3. PD-1 is Preferentially Upregulated on LILRB1 Negative CD8+ Cells

Since only PD-1 but not LILRB1 was upregulated on CD8+ T cells upon T cell activation, we next asked whether there is a difference in the upregulation of PD-1 on either LILRB1+ or LILRB1-CD8+ T cell subsets. PD-1 expression was upregulated preferentially from LILRB1-CD8+ T cells upon TCR stimulation (FIG. 3). Thus, it seems that expression of PD-1 and LILRB1 are mutually exclusive on CD8+ T cells, suggesting blocking PD-1 alone may not provide benefit on LILRB1+ effector CD8+ T cells, and combined effect of blocking both PD-1 and LILRB1 may be synergistic.

To confirm differential expression pattern of PD-1 and LILRB1 on different CD8+ subsets in human tumors, we evaluated the expression of LILRB1 and PD-1 on CD8+ T cells isolated from NSCLC tumor tissues by FACS. We observed different expression patterns of these two markers in these tumor samples (FIG. 4A). While some NSCLC tumors had CD8+ T cells dominantly expressing either PD-1 or LILRB1, other tumors contained similar numbers of LILRB1+ or PD-1+CD8+ cells (FIG. 4B). Nevertheless, in all the cases we examined, PD-1 and LILRB1 showed mutually exclusive expression pattern consistent with the data from healthy donor PBMCs. Importantly, LILRB1+CD8+ T cells were dominant in 5 out of 12 NSCLC tumors (FIG. 4B), suggesting LILRB1 blockade might be beneficial to these patients.

Furthermore, we analyzed publicly available single cell RNA sequencing data from HCC. In both datasets, LILRB1 and PD-1 showed mutually exclusive expression pattern in tumor CD8+ T cells (see, e.g., FIGS. 4A-4B). In contrast, expression patterns of other inhibitory receptors, such as TIGIT, TIM3, and CTLA-4, were largely overlapping with PD-1 (FIGS. 5A-5B).

More importantly, when tumor infiltrating CD8+ T cells were isolated and used as effector T cells in a MART-1 BiTE® molecule-mediated cytotoxicity assay, we found that HLA-G overexpression on target tumor cells significantly inhibited BiTER molecule-induced T cell activation (FIG. 4C) and cytolytic activity (FIG. 4D). Blockade of LILRB1 dramatically enhanced tumor CD8+ T cell effector function to kill HLA-G⁺ target cells (FIG. 4D). Our results demonstrated that LILRB1 is a checkpoint inhibitor for tumor infiltrating effector CD8+ T cells. Blocking LILRB1 may enhance CD8+ T cell cytolytic activity against tumor cells in patients.

### Materials and Methods

Human tumor dissociation. All human tumor specimens were collected under Institutional Review Board approval with appropriate informed consent. In all cases, materials obtained were surplus to standard clinical practice. Patient identity and PHI/identifying information were redacted from tissues and clinical data.

Lung tumor tissues were mechanically disrupted and incubated with the Liberase TL (Roche, 200 ug/ml) and DNase I (Roche, 20 U/ml): 1× MACS program h_tumor_1 using gentleMACS™ Octo Dissociator (Miltenyi Biotec), incubation at 37 C for 15 min, and then 1× MACS program h_tumor_02 to facilitate the release of lymphocytes from tissue. After the incubation, the tissue was passed through a 70 um filter to obtain a single cell suspension. The disaggregated tissues were centrifuge at 1500 rpm for 10 min and were re-suspended in DMEM/F12 containing 10% FBS (Life Technology, Carlsbad, CA, USA). Cells were blocked in FACS blocking buffer (1×PBS containing 5% FBS (Life Technology, Carlsbad, CA, USA) with 10 μg/ml each of normal human IgG (Rockland) and mouse IgG (Rockland), followed by antibody staining as described above.

### Example 4. Synergistic Effects of Anti-PD-1 and Anti-LILRB1 Treatment

We discovered that there are certain patient sub-populations that do not respond to anti-PD-1 treatment in vitro. As shown in FIG. 6A-6D, anti-PD-1 increased CD8+ T cell activity in about 70% of donor pairs in mixed lymphocyte reaction assays, of which 3 out 5 PD-1 responding pairs also respond to anti-LILRB1 (FIGS. 6A and 6D). More importantly, anti-LILRB1 showed activity to enhance T cell function in mixed lymphocyte reaction assays in those 2 donor pairs that did not respond to anti-PD-1 (FIG. 6C).

We next performed an MLR assay to compare effects of anti-LILRB1 or anti-PD-1 blockade alone or in combination on T cell activation. FIGS. 7A-7B shows MLR assay with isolated total human T cells and allogenic DCs in the presence of indicated blocking antibodies. Notable, in case of donor Pair #1, responses to anti-PD-1 alone, or anti-LILRB1 alone (as measured by IFNγ) were poor, each comparable to the control antibody. In stark contrast, response to the combined anti-PD-1 and anti-LILRB1 resulted in a significant increase in IFNγ production. Our results show that combination of anti-PD-1 and anti-LILRB1 blockade significantly increased IFNγ production in an MLR assay compared to the effect of single agent treatment (see, e.g., FIGS. 7A-7B, additional data not shown). Thus, blocking both pathways may further increase CD8+ T cell function.

In conclusion, we discovered that anti-PD-1 and anti-LILRB1 synergistically activated T cells in mixed lymphocyte reaction. Blocking both pathways significantly enhanced T cell activity and IFNγ production than blocking each pathway alone.

### Materials and Methods

Mixed lymphocyte reaction. Isolated human total T cells or CD8+ cells were seeded into round-bottom 96-well plate (Corning) at 105 per well together with 2×10⁴ irradiated allogeneic CD11c⁺ cell or MDDCs. When indicated, 10 μg/ml of anti-PD-1 (clone EH12.2H7, Biolegend) or anti-LILRB1 (clone GHI/75, Biolegend) were added to the culture. As control, 10 μg/ml of each matched isotype antibodies were used as control. Day 5 supernatants were collected and IFNγ level was determined by ELISA (BD Bioscience).

### Example 5. Anti-PD1 Therapy Increases LILRB1 Expression on Effector CD8 T Cells in Syngeneic Tumor Model

Mice or rats do not have a LILRB1 orthologue. To enable the assessment of the anti-PD1 checkpoint blockade treatment effects on LILRB1-expressing effector CD8 T cells in tumor, a LILRB1 transgenic mouse was generated by inserting a bacterial artificial chromosome (BAC) construct containing complete human LILRB1 gene into mouse genome. The resulting founders were confirmed to carry LILRB1 transgene by whole genome sequencing. A flow-activated cell sorting (FACS) analysis of peripheral blood mononuclear cells (PBMCs) obtained from transgenic mice confirmed that the expression of LILRB1 in subsets of immune cells from transgenic mice were similar to human LILRB1-expressing cells obtained from a healthy donor (data not shown).

Wild-type and transgenic mice were characterized in a phenotyping study and through clinical observations, body weights, clinical pathology, organ weights, and histopathology evaluation. Naïve transgenic mice phenocopied their wild-type littermates. No clinical observations, changes in body weight, or differences in clinical pathology parameters, organ weights, or histopathology were observed in any of the transgenic mice compared with wild-type mice. No differences were observed between wild-type and transgenic mice of the same sex in absolute numbers for the following immune cells in spleen and whole blood: CD4+ T cells, CD8+ T cells, B cells, natural killer cells, dendritic cells, monocytes, and macrophages.

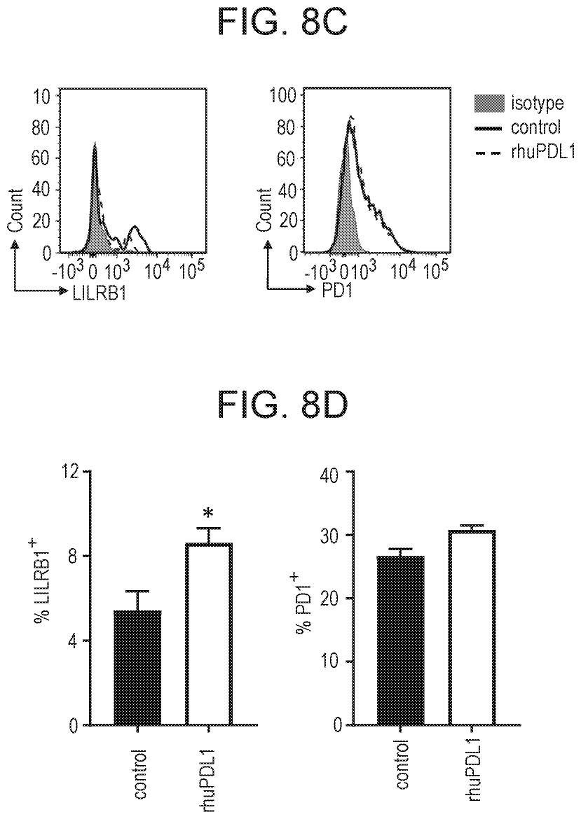

In an in vivo study, when MC38 tumor cells overexpressing membrane ovalbumin (as a tumor-specific antigen (MC38.OVA)) were implanted in transgenic mice, OVA-specific tumor infiltrating CD8+ T cells expressed LILRB1 (FIGS. 9A-9B). This suggests that LILRB1+CD8 T cells are reactive to tumor antigen. Furthermore, tumor infiltrating LILRB1+CD8 T cells also express CX3CR1, perforin and IFNγ, as seen for LILRB1+CD8 T cells in human tumors (Zheng, C. et al., 2017, Cell 169:1342-1356.e1316; Guo, X. et al., 2018, Nat Med 24:978-985).

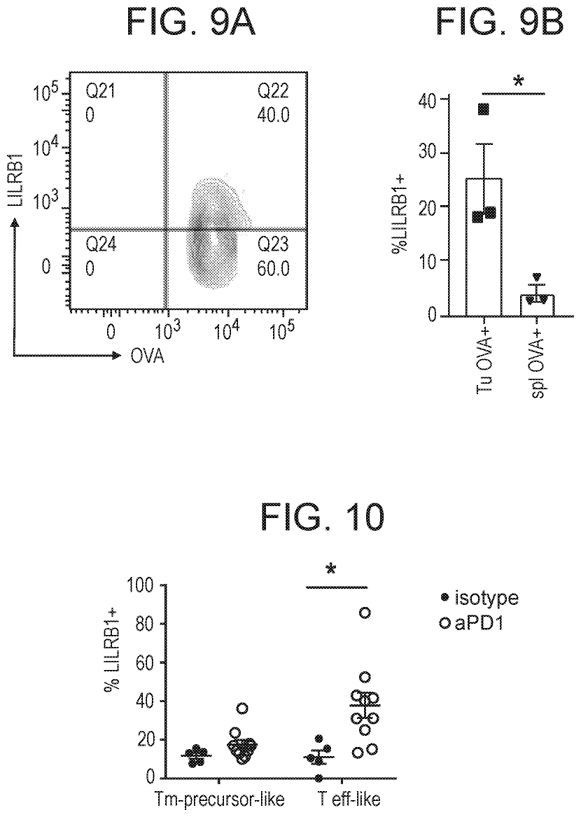

To assess if anti-PD1 blockade affect LILRB1+ T cells in tumor, tumor bearing LILRB1 transgenic mice were treated with anti-PD1 blocking antibody or isotype control antibody and the phenotypes of tumor infiltrating CD8 T cells were analyzed by flow cytometry. Our results showed that anti-PD1 significantly increased LILRB1+ effector CD8 T cells in tumor (FIG. 10; see also, Kurtulus S. et al., 2019, Immunity 50:181-194).

The in vivo data described herein support the concept that checkpoint blockade therapy (such as anti-PD-1) increases LILRB1 expression on effector CD8 T cells in tumor.

### Materials and Methods

In vivo tumor study. All animal experiments were performed under protocols approved by the Institutional Animal Care and Use Committee (IACUC) of Amgen, Inc. All mice were sourced from Charles River Laboratories (Hollister, CA site) and were provided water and chow ad libitum and maintained in a pathogen-free facility. Mice used in syngeneic tumor experiments were 6-8 weeks of age at the time implant. MC38 cells overexpressing ovalbumin or HLA-G in serum-free RPMI were inoculated in the right flank at 3×10⁵ cells per implant and allowed to grow for 10-14 days. For antibody treatment, the mice were then randomized by tumor volume (50-100 mm³) and treated with anti-PD1 antibody or isotype control antibody (rat IgG2a) at 15 mg/kg intraperitoneally. Tumors and spleens were taken down 48 h post-treatment for pharmacodynamic studies.

Tissue dissociation. Tumors were mechanically disrupted and digested with Collagenase D (Roche, 0.5 mg/ml) and DNase I (Roche, 40 U/ml). Disruption was performed as follows: 1× MACS program h_tumor_03 using gentleMACS Octo Dissociator (Miltenyi Biotec), followed by incubation at 37° C. for 15 min, and further disruption by 2×MACS program h_tumor_02. Spleenocytes were prepared using m_spleen_01 program on gentleMACS Octo Dissociator (Miltenyi Biotec) following by lysing blood cells with ACK buffer (Thermo Fisher). After disruption, cells were passed through a 70 μm filter and washed with RPMI containing 10% FBS. The single cell suspension was centrifuged at 1,500 rpm for 5 min and resuspended in FACS blocking buffer.

Transgenic mice generation. Human LILRB1 gene containing BAC DNA were microinjected into C57BL/6J single cell embryos, followed by embryo transfer to pseudopregnant female animals to generate 4 LILRB1-BAC transgenic founders (Horizon Discovery). Lines #5 and #15 were chosen for further characterization based on flow cytometry analysis results for LILRB1 expression in peripheral mononuclear cells in these founders. Transgene insertion was confirmed using genomic DNA prepared from tail using alkaline lysis by PCR and sequencing. Primers designed to both the 5′ end and 3′ end of the BAC construct was used to PCR amplify the LILRB1-BAC. 5′ end (Forward: 5′-AACCCACCTGACTTGGATCA-3′ and Reverse: 5′-TGAGGTATATGCAATCGTCAAAA-3′ (SEQ ID Nos. 32, 33) and 3′ end (Forward primer 5′-CTGGAACTGGTGGTGACAGA-3′ and Reverse primer-5′-TCTCCTCTAGGTGGTCAGCC-3′) (SEQ ID Nos. 34, 35).

### Example 6. Generation and Characterization of Anti-LILRB1 Antibodies

### 6.1 Generation of Anti-LILRB1 Antibodies

Mouse Strains. Fully human antibodies to human LILRB1 were generated by immunizing XENOMOUSE® transgenic mice (U.S. Pat. Nos. 6,114,598; 6,162,963; 6,833,268; 7,049,426; 7,064,244, which are incorporated herein by references in their entirety; Green et al., 1994, Nature Genetics 7:13-21; Mendez et al., 1997, Nature Genetics 15:146-156; Green and Jakobovitis, 1998, J. Ex. Med, 188:483-495; Kellerman and Green, Current Opinion in Biotechnology 13, 593-597, 2002). Animals from the XMG4-K and XMG4-KL XENOMOUSE® strains were used for these immunizations.

Immunizations. Multiple immunogens and routes of immunization were used to generate anti-human LILRB1 immune responses. For soluble recombinant protein immunizations, mice were immunized with alternating soluble human LILRB1-Fc and cynomolgus LILRB2-Fc. For cell-based immunizations, CHO-S cells were transiently transfected with either wild type human LILRB1 or cynomolgus LILRB2 as a source of immunogen. Animals were immunized with either of these transiently transfected CHO cells. Animals with the highest antigen-specific serum native titers directed against human LILRB1 and cynomolgus LILRB2 were used for hybridoma generation (Kohler and Milstein, 1975).

Hybridoma Generation. Animals exhibiting suitable serum titers were identified and lymphocytes were obtained from spleen and/or draining lymph nodes. Pooled lymphocytes (from each harvest) were dissociated from lymphoid tissue by grinding in a suitable medium (for example, Dulbecco's Modified Eagle Medium (DMEM); Invitrogen, Carlsbad, CA). B cells were selected and/or expanded using standard methods, and fused with a suitable fusion partner using techniques that were known in the art. Hybridoma supernatants with binding to human LILRB1 but no binding to human LILRA1 and human LILRA2 were then selected for further characterization.

Sequencing of LILRB1 Antagonist Antibodies. RNA (total or mRNA) was purified from wells containing the LILRB1 antagonist antibody-producing hybridoma cells using a Qiagen RNeasy mini or the Invitrogen mRNA catcher plus kit. Purified RNA was used to amplify the antibody heavy and light chain variable region (V) genes using cDNA synthesis via reverse transcription, followed by a polymerase chain reaction (RT-PCR). The fully human antibody gamma heavy chain was obtained using the Qiagen One Step Reverse Transcriptase PCR kit (Qiagen). The fully human kappa light chain was obtained using the Qiagen One Step Reverse Transcriptase PCR kit (Qiagen). Amino acid sequences were deduced from the corresponding nucleic acid sequences bioinformatically. The derived amino acid sequences were then analyzed to determine the germline sequence origin of the antibodies and to identify deviations from the germline sequence. The amino acid sequences corresponding to complementary determining regions (CDRs) of the sequenced antibodies were aligned and these alignments were used to group the clones by similarity.

Sequences of three exemplary anti-LILRB1 antibodies, 3C1, 30A10, and 19D6, are shown in the Sequence Table.

### 6.2 Functional Inhibition of Human LILRB1 Signaling

Jurkat cells stably expressing human LILRB1 and NFAT-luciferase reporter were used. For potency determination, hybridoma supernatant samples were serially titrated 3-fold in assay media and used to treat human LILRB1 reporter cells. Potency values for exemplary anti-LILRB1 antibodies from two repeat assays (n=1 and n=2) are shown in Table 1.

TABLE 1

| | Potency (nM) | |
|---|---|---|
| Antibody | n = 1 | n = 2 |
| 3C1 | 0.040 | 0.024 |
| 19D6 | 0.084 | 0.095 |
| 30A10 | 0.062 | 0.042 |

The binding of hybridoma supernatants to LILRB1 expressed by primary human were tested by flow cytometry and the results are summarized in Table 2.

TABLE 2

| Antibody | FACS GeoMean, test 1 | Antibody | FACS GeoMean test 2 |
|---|---|---|---|
| 3C1 | 1753 | 19D6 | 2538 |
| Isotype | 73 | 30A10 | 2536 |
| | | Isotype | 359 |

### 6.3 LILRB1 Binding Domain Determination

To determine the LILRB1 domains to which antibody in hybridoma supernatant sample was binding to, HEK 293T cells transiently expressing domain truncated LILRB1 constructs were used in flow cytometry based binding assay. Full length or domain truncated human LILRB1 proteins were expressed on host HEK 293T cells by transfection using cDNA expression vectors. Transfected cells were cultured for overnight and then used to determine which LILRB1 domain the antibodies were binding to. Hybridoma supernatants of a specific antibody was added to 96-well plate. Each hybridoma supernatant sample was tested for binding to each of the full length and truncated LILRB1 protein expressed on HEK 293T. The binding data for each sample were then used to determine the LILRB1 domain to which it was binding.

TABLE 3a

| | | Amino acid | |
|---|---|---|---|
| | | residues of | |
| Deleted | Remaining | remaining | |
| domains of | LILRB1 | LILRB1 | |
| LILRB1 | domains | domains | Description |
| — | D1, D2, D3, | 24-651 | Full length |
| | D4, TM, ICD | (SEQ ID | human |
| | | NO: 3) | LILRB1 |
| D1 | D2, D3, D4, | 116-651 | Human |
| | TM, ICD | (SEQ ID | LILRB1 |
| | | NO: 3) | without D1 |
| D1, D2 | D3, D4, TM, | 222-651 | Human |
| | ICD | (SEQ ID | LILRB1 |
| | | NO: 3) | without D1 |
| | | | and D2 |
| D1, D2, D3 | D4, TM, ICD | 313-651 | Human |
| | | (SEQ ID | LILRB1 |
| | | NO: 3) | without D1, |
| | | | D2 and D3 |

TABLE 3b

| | antibody |
|---|---|
| | binding |
| antibody | domain |
| 3C1 | D4 |
| 19D6 | D4 |
| 30A10 | D3 |

### 6.4 Relative Epitope Binning/Profiling

A common way to characterize epitopes is through competition experiments. Antibodies that compete with each other can be thought of as binding the same or overlapping site on the target. This example describes a method of determining competition for binding to human LILRB1.

Binning experiments can be conducted in a number of ways, and the method employed may have an effect on the assay results. Common to these methods is that LILRB1 is typically bound by one reference antibody and probed by another. If the reference antibody prevents the binding of the probe antibody then the antibodies are said to be in the same bin. The order in which the antibodies are employed is also relevant. If antibody A is employed as the reference antibody and blocks the binding of antibody B the converse is not always true: antibody B used as the reference antibody will not necessarily block antibody A. There are a number of factors in play here: the binding of an antibody can cause conformational changes in the target which prevent the binding of the second antibody, or epitopes which overlap but do not completely occlude each other may allow for the second antibody to still have enough high-affinity interactions with the target to allow binding. In general, if competition is observed in either order the antibodies are said to bin together, and if both antibodies can block each other then it is likely that the epitopes overlap more completely.

In this example, a modified antibody-antibody competition assay was used to determine the relative epitope binning profiles of the LILRB1 specific antibodies in a high throughput manner. Briefly, individual antibodies were tested for their ability to compete for binding with a panel of reference antibodies chosen based on their different binding characteristics (e.g., ligand blocking, domain binding, etc.) and primary sequences. The pattern of competition/binding of each test antibody with the reference antibody panel was then determined and compared to those produced from the other test antibodies. The degree of correlation between the individual test antibody competition/binding profiles was then compared. Antibodies that showed similar competition/binding profiles were binned (grouped) together (e.g., Binning Profile A, B, etc.).

Biotinylated recombinant soluble human LILRB1 protein was coupled to streptavidin coated, uniquely barcoded LumAvidin Beads® for 30 minutes in the dark at room temperature and washed twice. The reference antibody hybridoma supernatant samples were incubated with the antigen-coated beads for 1 hour in the dark at room temperature and washed three times. Beads were resuspended in FACS buffer containing Stabilguard® (SurModics). The antigen-coated, reference antibody-bound beads were pooled and then divided into individual sample wells containing a normalized (5 μg/ml) test antibody (hybridoma supernatant) sample (or negative control), incubated for 1 hour in the dark at room temperature and washed twice. The samples were then incubated with mouse anti-human IgG 2, 3, 4 antibody and incubated for 30 minutes in the dark at room temperature and washed twice. Antibodies bound to LILRB1 protein were then detected by incubating the samples with Alexa Fluor® 488 mouse IgG fragment-specific detection antibody for 15 minutes in the dark at room temperature, washed once and resuspended in FACS buffer. Samples were analyzed using an BD Accuri™ Flow Cytometer and an Intellicyt HyperCyt autoSampler.

To determine the antibody competition/binding profiles of the individual test antibodies, the reference-only antibody binding signal was subtracted from the reference plus test antibody signal for each competition/binding reaction (ie. across the entire reference antibody set). An individual antibody binding profile was defined as the collection of net binding values for each competition/binding reaction. The degree of similarity between individual profiles was then assessed by calculating the coefficient of determination between each of the test antibody profiles. Test antibodies showing high degrees of similarity (R2>0.8) to each other were then grouped into common binning profiles. Separate binning profiles were only defined if there were two or more samples with a high degree of correlation. If individual unique antibody binning profiles were observed (ie. they displayed a low degree of similarity to other test antibody binding profiles), the bin was classified as unknown. Using this method, the LILRB1 binding antibodies were sub-divided into unique binning profiles (A, B, C, etc.) (Table 4).

TABLE 4

| Antibody ID | Epitope BIN |
|---|---|
| 3C1 | C |
| 19D6 | C |
| 30A10 | C |

### 6.5 Affinities of the Anti-LILRB1 Antibodies

Affinity was measured on ForteBio Octet HTX (Molecular Devices) using SA biosensors (cat 18-5010) using assay buffer comprised of 10 mM Tris, 0.1% TritonX, 150 mM NaCl, 1 mM CaCl₂), 1 mg/mL BSA, pH 7.4. Assay was conducted at 25° C. and 1:1 model was used to fit the curves. Biotinylated goat anti-human Fc at 0.75 μg/mL was captured on SA sensors and antibodies as unpurified but quantified hybridoma supernatant were loaded on the sensor at 2 μg/mL for 600 seconds. Recombinant human LILRB1 (R&D Systems) titrated 1:2 from 25 nM was allowed to bind for 5 minutes, then dissociation was measured for 20 minutes. The results are shown in Table 5.

TABLE 5

| Antibody | K_{D} × 10⁻⁹ (M) | kₒₙ(1/Ms) | K_{dis}(1/s) |
|---|---|---|---|
| 3C1 | 1.68E−10 | 1.47E+06 | 2.47E−04 |
| 19D6 | 1.76E−10 | 1.17E+06 | 2.06E−04 |
| 30A10 | 1.03E−10 | 1.17E+06 | 1.20E−04 |

SEQUENCES

| SEQ | | |
|---|---|---|
| ID | Description | sequence |
| 1 | Q15116\|PDCD1_HUMAN | MQIPQAPWPVVWAVLQLGWRPGWELDSPDRPWNPPTESPALLVVTEGDNATFT |
| | Programmed | CSFSNTSESFVLNWYRMSPSNQTDKLAAFPEDRSQPGQDCRFRVTQLPNGRDF |
| | cell death | HMSVVRARRNDSGTYLCGAISLAPKAQIKESLRAELRVTERRAEVPTAHPSPS |
| | protein 1 | PRPAGQFQTLVVGVVGGLLGSLVLLVWVLAVICSRAARGTIGARRTGQPLKED |
| | (PD-1) | PSAVPVFSVDYGELDFQWREKTPEPPVPCVPEQTEYATIVFPSGMGTSSPARR |
| | | GSADGPRSAQPLRPEDGHCSWPL |
| 2 | Q9NZQ7\|PD- | MRIFAVFIFMTYWHLLNAFTVTVPKDLYVVEYGSNMTIECKFPVEKQLDLAAL |
| | 1L1_HUMAN | IVYWEMEDKNIIQFVHGEEDLKVQHSSYRQRARLLKDQLSLGNAALQITDVKL |
| | Programmed cell | QDAGVYRCMISYGGADYKRITVKVNAPYNKINQRILVVDPVTSEHELTCQAEG |
| | death 1 ligand | YPKAEVIWTSSDHQVLSGKTTTTNSKREEKLENVTSTLRINTTTNEIFYCTER |
| | 1 (PD-L1) | RLDPEENHTAELVIPELPLAHPPNERTHLVILGAILLCLGVALTFIFRLRKGR |
| | | MMDVKKCGIQDTNSKKQSDTHLEET |
| 3 | Q8NHL6\|LIRB1_HUMAN | MTPILTVLICLGLSLGPRTHVQAGHLPKPTLWAEPGSVITQGSPVTLRCQGGQ |
| | Leukocyte | ETQEYRLYREKKTALWITRIPQELVKKGQFPIPSITWEHAGRYRCYYGSDTAG |
| | immunoglobulin- | RSESSDPLELVVTGAYIKPTLSAQPSPVVNSGGNVILQCDSQVAFDGFSLCKE |
| | like receptor | GEDEHPQCLNSQPHARGSSRAIFSVGPVSPSRRWWYRCYAYDSNSPYEWSLPS |
| | subfamily B | DLLELLVLGVSKKPSLSVQPGPIVAPEETLTLQCGSDAGYNRFVLYKDGERDF |
| | member 1 | LQLAGAQPQAGLSQANFTLGPVSRSYGGQYRCYGAHNLSSEWSAPSDPLDILI |
| | (LILRB1) | AGQFYDRVSLSVQPGPTVASGENVTLLCQSQGWMQTFLLTKEGAADDPWRLRS |
| | | TYQSQKYQAEFPMGPVTSAHAGTYRCYGSQSSKPYLLTHPSDPLELVVSGPSG |
| | | GPSSPTTGPTSTSGPEDQPLTPTGSDPQSGLGRHLGVVIGILVAVILLLLLLL |
| | | LLFLILRHRRQGKHWTSTQRKADFQHPAGAVGPEPTDRGLQWRSSPAADAQEE |
| | | NLYAAVKHTQPEDGVEMDTRSPHDEDPQAVTYAEVKHSRPRREMASPPSPLSG |
| | | EFLDTKDRQAEEDRQMDTEAAASEAPQDVTYAQLHSLTLRREATEPPPSQEGP |
| | | SPAVPSIYATLAIH |
| 4 | Q8NHL6- | MTPILTVLICLGLSLGPRTHVQAGHLPKPTLWAEPGSVITQGSPVTLRCQGGQ |
| | 2\|LIRB1_HUMAN | ETQEYRLYREKKTALWITRIPQELVKKGQFPIPSITWEHAGRYRCYYGSDTAG |
| | Isoform 2 of | RSESSDPLELVVTGAYIKPTLSAQPSPVVNSGGNVILQCDSQVAFDGESLCKE |
| | Leukocyte | GEDEHPQCLNSQPHARGSSRAIFSVGPVSPSRRWWYRCYAYDSNSPYEWSLPS |
| | immunoglobulin- | DLLELLVLGVSKKPSLSVQPGPIVAPEETLTLQCGSDAGYNRFVLYKDGERDF |
| | like receptor | LQLAGAQPQAGLSQANFTLGPVSRSYGGQYRCYGAHNLSSEWSAPSDPLDILI |
| | subfamily B | AGQFYDRVSLSVQPGPTVASGENVTLLCQSQGWMQTELLTKE |
| | member 1 | GAADDPWRLRSTYQSQKYQAEFPMGPVTSAHAGTYRCYGSQSSKPYLLTHPSD |
| | OS = Homo sapiens | PLELVVSGPSGGPSSPTTGPTSTSAGPEDQPLTPTGSDPQSGLGRHLGVVIGI |
| | OX = 9606 | LVAVILLLLLLLLLFLILRHRRQGKHWTSTQRKADFQHPAGAVGPEPTDRGLQ |
| | GN = LILRB1 | WRSSPAADAQEENLYAAVKHTQPEDGVEMDTRSPHDEDPQAVTYAEVKHSRPR |
| | | REMASPPSPLSGEFLDTKDRQAEEDRQMDTEAAASEAPQDVTYAQLHSLTLRR |
| | | EATEPPPSQEGPSPAVPSIYATLAIH |
| 5 | Q8NHL6- | MTPILTVLICLGLSLGPRTHVQAGHLPKPTLWAEPGSVITQGSPVTLRCQGGQ |
| | 3\|LIRB1_HUMAN | ETQEYRLYREKKTALWITRIPQELVKKGQFPIPSITWEHAGRYRCYYGSDTAG |
| | Isoform 3 of | RSESSDPLELVVTGAYIKPTLSAQPSPVVNSGGNVILQCDSQVAFDGFSLCKE |
| | Leukocyte | GEDEHPQCLNSQPHARGSSRAIFSVGPVSPSRRWWYRCYAYDSNSPYEWSLPS |
| | immunoglobulin- | DLLELLVLGVSKKPSLSVQPGPIVAPEETLTLQCGSDAGYNRFVLYKDGERDF |
| | like receptor | LQLAGAQPQAGLSQANFTLGPVSRSYGGQYRCYGAHNLSSEWSAPSDPLDILI |
| | subfamily B | AGQFYDRVSLSVQPGPTVASGENVTLLCQSQGWMQTFLLTKEGAADDPWRLRS |
| | member 1 | TYQSQKYQAEFPMGPVTSAHAGTYRCYGSQSSKPYLLTHPSDPLELVVSGPSG |
| | OS = Homo sapiens | GPSSPTTGPTSTSAGPEDQPLTPTGSDPQSGLGRHLGVVIGILVAVILLLLLL |
| | OX = 9606 | LLLFLILRHRRQGKHWTSTQRKADFQHPAGAVGPEPTDRGLQWRSSPAADAQE |
| | GN = LILRB1 | ENLYAAVKHTQPEDGVEMDTRQSPHDEDPQAVTYAEVKHSRPRREMASPPSPL |
| | | SGEFLDTKDRQAEEDRQMDTEAAASEAPQDVTYAQLHSLTLRREATEPPPSQE |
| | | GPSPAVPSIYATLAIH |
| 6 | Q8NHL6- | MTPILTVLICLGLSLGPRTHVQAGHLPKPTLWAEPGSVITQGSPVTLRCQGGQ |
| | 4\|LIRB1_HUMAN | ETQEYRLYREKKTALWITRIPQELVKKGQFPIPSITWEHAGRYRCYYGSDTAG |
| | Isoform 4 of | RSESSDPLELVVTGAYIKPTLSAQPSPVVNSGGNVILQCDSQVAFDGFSLCKE |
| | Leukocyte | GEDEHPQCLNSQPHARGSSRAIFSVGPVSPSRRWWYRCYAYDSNSPYEWSLPS |
| | immunoglobulin- | DLLELLVLGVSKKPSLSVQPGPIVAPEETLTLQCGSDAGYNRFVLYKDGERDF |
| | like receptor | LQLAGAQPQAGLSQANFTLGPVSRSYGGQYRCYGAHNLSSEWSAPSDPLDILI |
| | subfamily B | AGQFYDRVSLSVQPGPTVASGENVTLLCQSQGWMQTFLLTKEGAADDPWRLRS |
| | member 1 | TYQSQKYQAEFPMGPVTSAHAGTYRCYGSQSSKPYLLTHPSDPLELVVSGPSG |
| | OS = Homo sapiens | GPSSPTTGPTSTSGPEDQPLTPTGSDPQSGLGRHLGVVIGILVAVILLLLLLL |
| | OX = 9606 | LLFLILRHRRQGKHWTSTQRKADFQHPAGAVGPEPTDRGLQWRSSPAADAQEE |
| | GN = LILRB1 | NLYAAVKHTQPEDGVEMDTRQSPHDEDPQAVTYAEVKHSRPRREMASPPSPLS |
| | | GEFLDTKDRQAEEDRQMDTEAAASEAPQDVTYAQLHSLTLRREATEPPPSQEG |
| | | PSPAVPSIYATLAIH |
| 7 | Q8NHL6- | MTPILTVLICLGLSLGPRTHVQAGHLPKPTLWAEPGSVITQGSPVTLRCQGGQ |
| | 5\|LIRB1_HUMAN | ETQEYRLYREKKTALWITRIPQELVKKGQFPIPSITWEHAGRYRCYYGSDTAG |
| | Isoform 5 of | RSESSDPLELVVTGAYIKPTLSAQPSPVVNSGGNVILQCDSQVAFDGFSLCKE |
| | Leukocyte | GEDEHPQCLNSQPHARGSSRAIFSVGPVSPSRRWWYRCYAYDSNSPYEWSLPS |
| | immunoglobulin- | DLLELLVLGVSKKPSLSVQPGPIVAPEETLTLQCGSDAGYNRFVLYKDGERDF |
| | like receptor | LQLAGAQPQAGLSQANFTLGPVSRSYGGQYRCYGAHNLSSEWSAPSDPLDILI |
| | subfamily B | AGQFYDRVSLSVQPGPTVASGENVTLLCQSQGWMQTFLLTKEGAADDPWRLRS |
| | member 1 | TYQSQKYQAEFPMGPVTSAHAGTYRCYGSQSSKPYLLTHPSDPLELVVSGPSG |
| | OS = Homo sapiens | GPSSPTTGPTSTSAGPEDQPLTPTGSDPQSGE |
| | OX = 9606 | |
| | GN = LILRB1 | |
| 8 | Q8N423\|LIRB2_HUMAN | MTPIVTVLICLGLSLGPRTHVQTGTIPKPTLWAEPDSVITQGSPVTLSCQGSL |
| | Leukocyte | EAQEYRLYREKKSASWITRIRPELVKNGQFHIPSITWEHTGRYGCQYYSRARW |
| | immunoglobulin- | SELSDPLVLVMTGAYPKPTLSAQPSPVVTSGGRVTLQCESQVAFGGFILCKEG |
| | like receptor | EEEHPQCLNSQPHARGSSRAIFSVGPVSPNRRWSHRCYGYDLNSPYVWSSPSD |
| | subfamily B | LLELLVPGVSKKPSLSVQPGPVVAPGESLTLQCVSDVGYDREVLYKEGERDLR |
| | member 2 | QLPGRQPQAGLSQANFTLGPVSRSYGGQYRCYGAHNLSSECSAPSDPLDILIT |
| | (LILRB2) | GQIRGTPFISVQPGPTVASGENVTLLCQSWRQFHTFLLTKAGAADAPLRLRSI |
| | | HEYPKYQAEFPMSPVTSAHAGTYRCYGSLNSDPYLLSHPSEPLELVVSGPSMG |
| | | SSPPPTGPISTPAGPEDQPLTPTGSDPQSGLGRHLGVVIGILVAVVLLLLLLL |
| | | LLFLILRHRRQGKHWTSTQRKADFQHPAGAVGPEPTDRGLQWRSSPAADAQEE |
| | | NLYAAVKDTQPEDGVEMDTRAAASEAPQDVTYAQLHSLTLRRKATEPPPSQER |
| | | EPPAEPSIYATLAIH |
| 9 | Q8N423- | MTPIVTVLICLGLSLGPRTHVQTGTIPKPTLWAEPDSVITQGSPVTLSCQGSL |
| | 2\|LIRB2_HUMAN | EAQEYRLYREKKSASWITRIRPELVKNGQFHIPSITWEHTGRYGCQYYSRARW |
| | Isoform 2 of | SELSDPLVLVMTGAYPKPTLSAQPSPVVTSGGRVTLQCESQVAFGGFILCKEG |
| | Leukocyte | EEEHPQCLNSQPHARGSSRAIFSVGPVSPNRRWSHRCYGYDLNSPYVWSSPSD |
| | immunoglobulin- | LLELLVPGVSKKPSLSVQPGPVVAPGESLTLQCVSDVGYDREVLYKEGERDLR |
| | like receptor | QLPGRQPQAGLSQANFTLGPVSRSYGGQYRCYGAHNLSSECSAPSDPLDILIT |
| | subfamily B | GQIRGTPFISVQPGPTVASGENVTLLCQSWRQFHTFLLTKAGAADAPLRLRSI |
| | member 2 | HEYPKYQAEFPMSPVTSAHAGTYRCYGSLNSDPYLLSHPSEPLELVVSGPSMG |
| | OS = Homo sapiens | SSPPPTGPISTPGPEDQPLTPTGSDPQSGLGRHLGVVIGILVAVVLLLLLLLL |
| | OX = 9606 | LFLILRHRRQGKHWTSTQRKADFQHPAGAVGPEPTDRGLQWRSSPAADAQEEN |
| | GN = LILRB2 | LYAAVKDTQPEDGVEMDTRAAASEAPQDVTYAQLHSLTLRRKATEPPPSQERE |
| | | PPAEPSIYATLAIH |
| 10 | Q8N423- | MTPIVTVLICLGLSLGPRTHVQTGTIPKPTLWAEPDSVITQGSPVTLSCQGSL |
| | 3\|LIRB2_HUMAN | EAQEYRLYREKKSASWITRIRPELVKNGQFHIPSITWEHTGRYGCQYYSRARW |
| | Isoform 3 of | SELSDPLVLVMTGAYPKPTLSAQPSPVVTSGGRVTLQCESQVAFGGFILCKEG |
| | Leukocyte | EEEHPQCLNSQPHARGSSRAIFSVGPVSPNRRWSHRCYGYDLNSPYVWSSPSD |
| | immunoglobulin- | LLELLVPGVSKKPSLSVQPGPVVAPGESLTLQCVSDVGYDREVLYKEGERDLR |
| | like receptor | QLPGRQPQAGLSQANFTLGPVSRSYGGQYRCYGAHNLSSECSAPSDPLDILIT |
| | subfamily B | GQIRGTPFISVQPGPTVASGENVTLLCQSWRQFHTFLLTKAGAADAPLRLRSI |
| | member 2 | HEYPKYQAEFPMSPVTSAHAGTYRCYGSLNSDPYLLSHPSEPLELVVSGPSMG |
| | OS = Homo sapiens | SSPPPTGPISTPAGPEDQPLTPTGSDPQSGLGRHLGVVIGILVAVVLLLLLLL |
| | OX = 9606 | LLFLILRHRRQGKHWTSSPAQLPTPRKKTSMLP |
| | GN = LILRB2 | |
| 11 | Q8N423- | MTGAYPKPTLSAQPSPVVTSGGRVTLQCESQVAFGGFILCKEGEEEHPQCLNS |
| | 4\|LIRB2_HUMAN | QPHARGSSRAIFSVGPVSPNRRWSHRCYGYDLNSPYVWSSPSDLLELLVPGVS |
| | Isoform 4 of | KKPSLSVQPGPVVAPGESLTLQCVSDVGYDRFVLYKEGERDLRQLPGRQPQAG |
| | Leukocyte | LSQANFTLGPVSRSYGGQYRCYGAHNLSSECSAPSDPLDILITGQIRGTPFIS |
| | immunoglobulin- | VQPGPTVASGENVTLLCQSWRQFHTFLLTKAGAADAPLRLRSIHEYPKYQAEF |
| | like receptor | PMSPVTSAHAGTYRCYGSLNSDPYLLSHPSEPLELVVSGPSMGSSPPPTGPIS |
| | subfamily | TPAGPEDQPLTPTGSDPQSGLGRHLGVVIGILVAVVLLLLLLLLLFLILRHRR |
| | member 2 | QGKHWTSTQRKADFQHPAGAVGPEPTDRGLQWRSSPAADAQEENLYAAVKDTQ |
| | OS = Homo sapiens | PEDGVEMDTRAAASEAPQDVTYAQLHSLTLRRKATEPPPSQEREPPAEPSIYA |
| | OX = 9606 | TLAIH |
| | GN = LILRB2 | |
| 12 | P17693\|HLAG_HUMAN | MVVMAPRTLFLLLSGALTLTETWAGSHSMRYFSAAVSRPGRGEPRFIAMGYVD |
| | HLA class I | DTQFVRFDSDSACPRMEPRAPWVEQEGPEYWEEETRNTKAHAQTDRMNLQTLR |
| | histocompatibility | GYYNQSEASSHTLQWMIGCDLGSDGRLLRGYEQYAYDGKDYLALNEDLRSWTA |
| | antigen, | ADTAAQISKRKCEAANVAEQRRAYLEGTCVEWLHRYLENGKEMLQRADPPKTH |
| | alpha chain G | VTHHPVFDYEATLRCWALGFYPAEIILTWQRDGEDQTQDVELVETRPAGDGTF |
| | | QKWAAVVVPSGEEQRYTCHVQHEGLPEPLMLRWKQSSLPTIPIMGIVAGLVVL |
| | | AAVVTGAAVAAVLWRKKSSD |
| 13 | P17693- | MVVMAPRTLFLLLSGALTLTETWAGSHSMRYFSAAVSRPGRGEPRFIAMGYVD |
| | 2\|HLAG_HUMAN | DTQFVRFDSDSACPRMEPRAPWVEQEGPEYWEEETRNTKAHAQTDRMNLQTLR |
| | Isoform 2 of | GYYNQSEAKPPKTHVTHHPVFDYEATLRCWALGFYPAEIILTWQRDGEDQTQD |
| | HLA class I | VELVETRPAGDGTFQKWAAVVVPSGEEQRYTCHVQHEGLPEPLMLRWKQSSLP |
| | histocompatibility | TIPIMGIVAGLVVLAAVVTGAAVAAVLWRKKSSD |
| | antigen, | |
| | alpha chain G | |
| | OS = Homo sapiens | |
| | OX = 9606 | |
| | GN = HLA-G | |
| 14 | P17693- | MVVMAPRTLFLLLSGALTLTETWAGSHSMRYFSAAVSRPGRGEPRFIAMGYVD |
| | 3\|HLAG_HUMAN | DTQFVRFDSDSACPRMEPRAPWVEQEGPEYWEEETRNTKAHAQTDRMNLQTLR |
| | Isoform 3 of | GYYNQSEAKQSSLPTIPIMGIVAGLVVLAAVVTGAAVAAVLWRKKSSD |
| | HLA class I | |
| | histocompatibility | |
| | antigen, | |
| | alpha chain G | |
| | OS = Homo sapiens | |
| | OX = 9606 | |
| | GN = HLA-G | |
| 15 | P17693- | MVVMAPRTLFLLLSGALTLTETWAGSHSMRYFSAAVSRPGRGEPRFIAMGYVD |
| | 4\|HLAG_HUMAN | DTQFVRFDSDSACPRMEPRAPWVEQEGPEYWEEETRNTKAHAQTDRMNLQTLR |
| | Isoform 4 of | GYYNQSEASSHTLQWMIGCDLGSDGRLLRGYEQYAYDGKDYLALNEDLRSWTA |
| | HLA class I | ADTAAQISKRKCEAANVAEQRRAYLEGTCVEWLHRYLENGKEMLQRAKQSSLP |
| | histocompatibility | TIPIMGIVAGLVVLAAVVTGAAVAAVLWRKKSSD |
| | antigen, | |
| | alpha chain G | |
| | OS = Homo sapiens | |
| | OX = 9606 | |
| | GN = HLA-G | |
| 16 | P17693- | MVVMAPRTLFLLLSGALTLTETWAGSHSMRYFSAAVSRPGRGEPRFIAMGYVD |
| | 5\|HLAG_HUMAN | DTQFVRFDSDSACPRMEPRAPWVEQEGPEYWEEETRNTKAHAQTDRMNLQTLR |
| | Isoform 5 of | GYYNQSEASSHTLQWMIGCDLGSDGRLLRGYEQYAYDGKDYLALNEDLRSWTA |
| | HLA class I | ADTAAQISKRKCEAANVAEQRRAYLEGTCVEWLHRYLENGKEMLQRADPPKTH |
| | histocompatibility | VTHHPVFDYEATLRCWALGFYPAEIILTWQRDGEDQTQDVELVETRPAGDGTF |
| | antigen, | QKWAAVVVPSGEEQRYTCHVQHEGLPEPLMLRWSKEGDGGIMSVRESRSLSED |
| | alpha chain G | L |
| | OS = Homo sapiens | |
| | OX = 9606 | |
| | GN = HLA-G | |
| 17 | P17693- | MVVMAPRTLFLLLSGALTLTETWAGSHSMRYFSAAVSRPGRGEPRFIAMGYVD |
| | 6\|HLAG_HUMAN | DTQFVRFDSDSACPRMEPRAPWVEQEGPEYWEEETRNTKAHAQTDRMNLQTLR |
| | Isoform 6 of | GYYNQSEAKPPKTHVTHHPVFDYEATLRCWALGFYPAEIILTWQRDGEDQTQD |
| | HLA class I | VELVETRPAGDGTFQKWAAVVVPSGEEQRYTCHVQHEGLPEPLMLRWSKEGDG |
| | histocompatibility | GIMSVRESRSLSEDL |
| | antigen, | |
| | alpha chain G | |
| | OS = Homo sapiens | |
| | OX = 9606 | |
| | GN = HLA-G | |
| 18 | P17693- | MVVMAPRTLFLLLSGALTLTETWAGSHSMRYFSAAVSRPGRGEPRFIAMGYVD |
| | 7\|HLAG_HUMAN | DTQFVRFDSDSACPRMEPRAPWVEQEGPEYWEEETRNTKAHAQTDRMNLQTLR |
| | Isoform 7 of | GYYNQSEASE |
| | HLA class I | |
| | histocompatibility | |
| | antigen, | |
| | alpha chain G | |
| | OS = Homo sapiens | |
| | OX = 9606 | |
| | GN = HLA-G | |
| 19 | 20C1 HC CDR1 | SYDMS |
| 20 | 20C1 HC CDR2 | LISGGGSQTYYAESVKG |
| 21 | 20C1 HC CDR3 | PSGHYFYAMDV |
| 22 | 20C1 LC CDR1 | RASQGISNWLA |
| 23 | 20C1 LC CDR2 | AASSLQS |
| 24 | 20C1 LC CDR3 | QQAESFPHT |
| 25 | 20C1 HC | EVQLLESGGGLVQPGGSLRLSCAASGFTFSSYDMSWVRQAPGKGLEWVSLISG |
| | VARIABLE | GGSQTYYAESVKGRFTISRDNSKNTLYLQMNSLRAEDTAVYFCASPSGHYFYA |
| | | MDVWGQGTTVTVSS |
| 26 | 20C1 LC | DIQMTQSPSSVSASVGDRVTITCRASQGISNWLAWYQQKPGKAPKLLIFAASS |
| | VARIABLE | LQSGVPSRFSGSGSGTDFTLTISSLQPEDFATYYCQQAESFPHTFGGGTKVEI |
| | | K |
| 27 | 20C1 HC FULL | MDMRVPAQLLGLLLLWLRGARCEVQLLESGGGLVQPGGSLRLSCAASGFTESS |
| | LENGTH | YDMSWVRQAPGKGLEWVSLISGGGSQTYYAESVKGRFTISRDNSKNTLYLQMN |
| | | SLRAEDTAVYFCASPSGHYFYAMDVWGQGTTVTVSSASTKGPSVFPLAPSSKS |
| | | TSGGTAALGCLVKDYFPEPVTVSWNSGALTSGVHTFPAVLQSSGLYSLSSVVT |
| | | VPSSSLGTQTYICNVNHKPSNTKVDKKV |
| | | EPKSCDKTHTCPPCPAPELLGGPSVFLFPPKPKDTLMISRTPEVTCVVVDVSH |
| | | EDPEVKFNWYVDGVEVHNAKTKPCEEQYGSTYRCVSVLTVLHQDWLNGKEYKC |
| | | KVSNKALPAPIEKTISKAKGQPREPQVYTLPPSREEMTKNQVSLTCLVKGFYP |
| | | SDIAVEWESNGQPENNYKTTPPVLDSDGSFFLYSKLTVDKSRWQQGNVFSCSV |
| | | MHEALHNHYTQKSLSLSPGK |
| 28 | 20C1 LC FULL | MDMRVPAQLLGLLLLWLRGARCDIQMTQSPSSVSASVGDRVTITCRASQGISN |
| | LENGTH | WLAWYQQKPGKAPKLLIFAASSLQSGVPSRFSGSGSGTDFTLTISSLQPEDFA |
| | | TYYCQQAESFPHTFGGGTKVEIKRTVAAPSVFIFPPSDEQLKSGTASVVCLLN |
| | | NFYPREAKVQWKVDNALQSGNSQESVTEQDSKDSTYSLSSTLTLSKADYEKHK |
| | | VYACEVTHQGLSSPVTKSENRGEC |
| 29 | 20C1 Heavy | ASTKGPSVFPLAPSSKSTSGGTAALGCLVKDYFPEPVTVSWNSGALTGVHTFP |
| | chain constant | AVLQSSGLYSLSSVVTVPSSSLGTQTYICNVNHKPSNTKVDKKVEPKSCDKTH |
| | region | TCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPA |
| | | PIEKTISKAKGQPREPQVYTLPPSREE |
| | | MTKNQVSLTCLVKGFYPSDIAVEWESNGQPENNYKTTPPVLDSDGSFFLYSKL |
| | | TVDKSRWQQGNVFSCSVMHEALHNHYTQKSLSLSPGK |
| 30 | 20C1 Light | RTVAAPSVFIFPPSDEQLKSGTASVVCLLNNFYPREAKVQWKVDNALQSGNSQ |
| | chain constant | ESVTEQDSKDSTYSLSSTLTLSKADYEKHKVYACEVTHQGLSSPVTKSENRGE |
| | region | C |
| 31 | siRNA targeting | GGCUGAACAAAGGAGAtt |
| | HLA-G | |
| 32 | Primer for | AACCCACCTGACTTGGATCA |
| | transgenic | |
| | mouse | |
| 33 | Primer for | TGAGGTATATGCAATCGTCAAAA |
| | transgenic | |
| | mouse | |
| 34 | Primer for | CTGGAACTGGTGGTGACAGA |
| | transgenic | |
| | mouse | |
| 35 | Primer for | TCTCCTCTAGGTGGTCAGCC |
| | transgenic | |
| | mouse | |
| 36 | 3C1 HC CDR1 | DYDMH |
| 37 | 3C1 HC CDR2 | TIDTAGDTYYPDSVRG |
| 38 | 3C1 HC CDR3 | DVPGTGFDH |
| 39 | 3C1 LC CDR1 | QGDSLRSYYES |
| 40 | 3C1 LC CDR2 | GKNNRPS |
| 41 | 3C1 LC CDR3 | NSRDNSGDHWV |
| 42 | 3C1 HC VARIABLE | EVQLVESGGGLVQPGGSLRLSCAASGFTFSDYDMHWVRQATGKGLEWVATIDT |
| | | AGDTYYPDSVRGRFTISRENAKNSLSLQMNSLRAGDTAVYFCARDVPGTGFDH |
| | | WGQGTLVTVSS |
| 43 | 3C1 LC VARIABLE | SSELTQDPAVSVALGQTVRITCQGDSLRSYYESWYQQKPGQAPVLVIYGKNNR |
| | | PSGIPDRFSGSISGNTASLTITGTQAEDEADYYCNSRDNSGDHWVFGGGTRLT |
| | | VL |
| 44 | 3C1 HC FULL | MDMRVPAQLLGLLLLWLRGARCEVQLVESGGGLVQPGGSLRLSCAASGFTFSD |
| | LENGTH | YDMHWVRQATGKGLEWVATIDTAGDTYYPDSVRGRFTISRENAKNSLSLQMNS |
| | | LRAGDTAVYFCARDVPGTGFDHWGQGTLVTVSSASTKGPSVFPLAPCSRSTSE |
| | | STAALGCLVKDYFPEPVTVSWNSGALTSGVHTFPAVLQSSGLYSLSSVVTVPS |
| | | SSLGTKTYTCNVDHKPSNTKVDKRVESKYGPPCPSCPAPEFLGGPSVFLFPPK |
| | | PKDTLMISRTPEVTCVVVDVSQEDPEVQFNWYVDGVEVHNAKTKPREEQENST |
| | | YRVVSVLTVLHQDWLNGKEYKCKVSNKGLPSSIEKTISKAKGQPREPQVYTLP |
| | | PSQEEMTKNQVSLTCLVKGFYPSDIAVEWESNGQPENNYKTTPPVLDSDGSFF |
| | | LYSRLTVDKSRWQEGNVFSCSVMHEALHNHYTQKSLSLSLGK |
| 45 | 3C1 LC FULL | MDMRVPAQLLGLLLLWLRGARCSSELTQDPAVSVALGQTVRITCQGDSLRSYY |
| | LENGTH | ESWYQQKPGQAPVLVIYGKNNRPSGIPDRESGSISGNTASLTITGTQAEDEAD |
| | | YYCNSRDNSGDHWVFGGGTRLTVLGQPKAAPSVTLFPPSSEELQANKATLVCL |
| | | ISDFYPGAVTVAWKADSSPVKAGVETTTPSKQSNNKYAASSYLSLTPEQWKSH |
| | | RSYSCQVTHEGSTVEKTVAPTECS |
| 46 | 3C1 Heavy chain | ASTKGPSVFPLAPCSRSTSESTAALGCLVKDYFPEPVTVSWNSGALTSGVHTF |
| | constant region | PAVLQSSGLYSLSSVVTVPSSSLGTKTYTCNVDHKPSNTKVDKRVESKYGPPC |
| | | PSCPAPEFLGGPSVFLFPPKPKDTLMISRTPEVTCVVVDVSQEDPEVQFNWYV |
| | | DGVEVHNAKTKPREEQFNSTYRVVSVLTVLHQDWLNGKEYKCKVSNKGLPSSI |
| | | EKTISKAKGQPREPQVYTLPPSQEEMTKNQVSLTCLVKGFYPSDIAVEWESNG |
| | | QPENNYKTTPPVLDSDGSFFLYSRLTVDKSRWQEGNVFSCSVMHEALHNHYTQ |
| | | KSLSLSLGK |
| 47 | 3C9 light chain | GQPKAAPSVTLFPPSSEELQANKATLVCLISDFYPGAVTVAWKADSSPVKAGV |
| | constant region | ETTTPSKQSNNKYAASSYLSLTPEQWKSHRSYSCQVTHEGSTVEKTVAPTECS |
| 48 | 30A10 HC CDR1 | SYAMH |
| 49 | 30A10 HC CDR2 | IIWFDGSQRYYADSVKG |
| 50 | 30A10 HC CDR3 | DELTGFDY |
| 51 | 30A10 LC CDR1 | RASQNIINFLN |
| 52 | 30A10 LC CDR2 | AASSLRS |
| 53 | 30A10 LC CDR3 | QQSFSTPLT |
| 54 | 30A10 HC | QVQLVESGGGVVQPGRSLRLSCAASGFTFSSYAMHWVRQAPGKGLEWVAIIWF |
| | VARIABLE | DGSQRYYADSVKGRFTISRDNSRDTLYLQMNSLRAEDTAVYYCARDELTGFDY |
| | | WGQGTLVTVSS |
| 55 | 30A10 LC | DIQMTQSPSSLSASVGDRVTISCRASQNIINFLNWYQQKPGKAPKVLIDAASS |
| | VARIABLE | LRSGVPSRFSGSGSETDESLTISSLQPEDFATYYCQQSFSTPLTFGGGTKVEI |
| | | R |
| 56 | 30A10 HC FULL | MDMRVPAQLLGLLLLWLRGARCQVQLVESGGGVVQPGRSLRLSCAASGFTESS |
| | LENGTH | YAMHWVRQAPGKGLEWVAIIWFDGSQRYYADSVKGRFTISRDNSRDTLYLQMN |
| | | SLRAEDTAVYYCARDELTGFDYWGQGTLVTVSSASTKGPSVFPLAPCSRSTSE |
| | | STAALGCLVKDYFPEPVTVSWNSGALTSGVHTFPAVLQSSGLYSLSSVVTVPS |
| | | SSLGTKTYTCNVDHKPSNTKVDKRVESKYGPPCPSCPAPEFLGGPSVFLFPPK |
| | | PKDTLMISRTPEVTCVVVDVSQEDPEVQFNWYVDGVEVHNAKTKPREEQENST |
| | | YRVVSVLTVLHQDWLNGKEYKCKVSNKGLPSSIEKTISKAKGQPREPQVYTLP |
| | | PSQEEMTKNQVSLTCLVKGFYPSDIAVEWESNGQPENNYKTTPPVLDSDGSFF |
| | | LYSRLTVDKSRWQEGNVFSCSVMHEALHNHYTQKSLSLSLGK |
| 57 | 30A10 LC FULL | MDMRVPAQLLGLLLLWLRGARCDIQMTQSPSSLSASVGDRVTISCRASQNIIN |
| | LENGTH | FLNWYQQKPGKAPKVLIDAASSLRSGVPSRFSGSGSETDESLTISSLQPEDFA |
| | | TYYCQQSFSTPLTFGGGTKVEIRRTVAAPSVFIFPPSDEQLKSGTASVVCLLN |
| | | NFYPREAKVQWKVDNALQSGNSQESVTEQDSKDSTYSLSSTLTLSKADYEKHK |
| | | VYACEVTHQGLSSPVTKSENRGEC |
| 46 | 30A10 Heavy | same as SEQ ID NO: 46 |
| | chain constant | ASTKGPSVFPLAPCSRSTSESTAALGCLVKDYFPEPVTVSWNSGALTSGVHTF |
| | region | PAVLQSSGLYSLSSVVTVPSSSLGTKTYTCNVDHKPSNTKVDKRVESKYGPPC |
| | | PSCPAPEFLGGPSVFLFPPKPKDTLMISRTPEVTCVVVDVSQEDPEVQFNWYV |
| | | DGVEVHNAKTKPREEQFNSTYRVVSVLTVLHQDWLNGKEYKCKVSNKGLPSSI |
| | | EKTISKAKGQPREPQVYTLPPSQEEMTKNQVSLTCLVKGFYPSDIAVEWESNG |
| | | QPENNYKTTPPVLDSDGSFFLYSRLTVDKSRWQEGNVFSCSVMHEALHNHYTQ |
| | | KSLSLSLGK |
| 30 | 30A10 light | same as SEQ ID NO: 30 |
| | chain constant | RTVAAPSVFIFPPSDEQLKSGTASVVCLLNNFYPREAKVQWKVDNALQSGNSQ |
| | region | ESVTEQDSKDSTYSLSSTLTLSKADYEKHKVYACEVTHQGLSSPVTKSENRGE |
| | | C |
| 58 | 19D6 HC CDR1 | DYDMH |
| 59 | 19D6 HC CDR2 | AIDTAGDTYYLGPVKG |
| 60 | 19D6 HC CDR3 | DSGSSFDY |
| 61 | 19D6 LC CDR1 | QGDSLRNYYES |
| 62 | 19D6 LC CDR2 | GKNNRPS |
| 63 | 19D6 LC CDR3 | MSRDSSGNHWV |
| 64 | 19D6 HC | EVQLVESGGGLVQPGESLRLSCAASGFTFSDYDMHWVRQATGKGLEWVSAIDT |
| | VARIABLE | AGDTYYLGPVKGRFTISRENAKNSLFLHMNSLRVGDTAVYYCIRDSGSSFDYW |
| | | GQGTLVTVSS |
| 65 | 19D6 LC | TSELTQDPAVSVALGQTVRITCQGDSLRNYYESWYQQKPGQAPVLVIYGKNNR |
| | VARIABLE | PSGIPDRFSGSSSGNTASLIITGTQAEDEADYYCMSRDSSGNHWVFGGGTKVT |
| | | VL |
| 66 | 19D6 HC FULL | MDMRVPAQLLGLLLLWLRGARCEVQLVESGGGLVQPGESLRLSCAASGFTFSD |
| | LENGTH | YDMHWVRQATGKGLEWVSAIDTAGDTYYLGPVKGRFTISRENAKNSLFLHMNS |
| | | LRVGDTAVYYCIRDSGSSFDYWGQGTLVTVSSASTKGPSVFPLAPCSRSTSES |
| | | TAALGCLVKDYFPEPVTVSWNSGALTSGVHTFPAVLQSSGLYSLSSVVTVPSS |
| | | SLGTKTYTCNVDHKPSNTKVDKRVESKYGPPCPSCPAPEFLGGPSVFLFPPKP |
| | | KDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 67 | 19D6 LC FULL | MDMRVPAQLLGLLLLWLRGARCTSELTQDPAVSVALGQTVRITCQGDSLRNYY |
| | LENGTH | ESWYQQKPGQAPVLVIYGKNNRPSGIPDRESGSSSGNTASLIITGTQAEDEAD |
| | | YYCMSRDSSGNHWVFGGGTKVTVLGQPKAAPSVTLFPPSSEELQANKATLVCL |
| | | ISDFYPGAVTVAWKADSSPVKAGVETTTPSKQSNNKYAASSYLSLTPEQWKSH |
| | | RSYSCQVTHEGSTVEKTVAPTECS |
| 46 | 19D6 Heavy | same as SEQ ID NO: 46 |
| | chain constant | ASTKGPSVFPLAPCSRSTSESTAALGCLVKDYFPEPVTVSWNSGALTSGVHTF |
| | region | PAVLQSSGLYSLSSVVTVPSSSLGTKTYTCNVDHKPSNTKVDKRVESKYGPPC |
| | | PSCPAPEFLGGPSVFLFPPKPKDTLMISRTPEVTCVVVDVSQEDPEVQFNWYV |
| | | DGVEVHNAKTKPREEQFNSTYRVVSVLTVLHQDWLNGKEYKCKVSNKGLPSSI |
| | | EKTISKAKGQPREPQVYTLPPSQEEMTKNQVSLTCLVKGFYPSDIAVEWESNG |
| | | QPENNYKTTPPVLDSDGSFFLYSRLTVDKSRWQEGNVFSCSVMHEALHNHYTQ |
| | | KSLSLSLGK |
| 47 | 19D6 light | same as SEQ ID NO: 47 |
| | chain constant | GQPKAAPSVTLFPPSSEELQANKATLVCLISDFYPGAVTVAWKADSSPVKAGV |
| | region | ETTTPSKQSNNKYAASSYLSLTPEQWKSHRSYSCQVTHEGSTVEKTVAPTECS |
| 68 | Immunoglobulin | ASTKGPSVFPLAPSSKSTSGGTAALGCLVKDYFPEPVTVSWNSGALTSGVHTF |
| | heavy constant | PAVLQSSGLYSLSSVVTVPSSSLGTQTYICNVNHKPSNTKVDKKVEPKSCDKT |
| | gamma 1 | HTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALP |
| | | APIEKTISKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVMHEALHNH |
| | | YTQKSLSLSPGK |
| 69 | Immunoglobulin | ASTKGPSVFPLAPCSRSTSESTAALGCLVKDYFPEPVTVSWNSGALTSGVHTF |
| | heavy constant | PAVLQSSGLYSLSSVVTVPSSNFGTQTYTCNVDHKPSNTKVDKTVERKCCVEC |
| | gamma 2 | PPCPAPPVAGPSVFLFPPKPKDTLMISRTPEVTCVVVDVSHEDPEVQFNWYVD |
| | | GVEVHNAKTKPREEQFNSTFRVVSVLTVVHQDWLNGKEYKCKVSNKGLPAPIE |
| | | KTISKTKGQPREPQVYTLPPSREEMTKNQVSLTCLVKGFYPSDISVEWESNGQ |
| | | PENNYKTTPPMLDSDGSFFLYSKLTVDKSRWQQGNVFSCSVMHEALHNHYTQK |
| | | SLSLSPGK |
| 70 | Immunoglobulin | ASTKGPSVFPLAPCSRSTSGGTAALGCLVKDYFPEPVTVSWNSGALTSGVHTF |
| | heavy constant | PAVLQSSGLYSLSSVVTVPSSSLGTQTYTCNVNHKPSNTKVDKRVELKTPLGD |
| | gamma 3 | TTHTCPRCPEPKSCDTPPPCPRCPEPKSCDTPPPCPRCPEPKSCDTPPPCPRC |
| | | PAPELLGGPSVFLFPPKPKDTLMISRTPEVTCVVVDVSHEDPEVQFKWYVDGV |
| | | EVHNAKTKPREEQYNSTFRVVSVLTVLHQDWLNGKEYKCKVSNKALPAPIEKT |
| | | ISKTKGQPREPQVYTLPPSREEMTKNQVSLTCLVKGFYPSDIAVEWESSGQPE |
| | | NNYNTTPPMLDSDGSFFLYSKLTVDKSRWQQGNIFSCSVMHEALHNRFTQKSL |
| | | SLSPGK |

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

1. An antibody, or antigen-binding fragment thereof, that binds LILRB1 and comprises:
(i) a heavy chain variable region (VH) that comprises:
(a) a VH CDR-H1 comprising the amino acid sequence of SEQ ID NO: 36, 48, or 58;
(b) a VH CDR-H2 comprising the amino acid sequence of SEQ ID NO: 37, 49, or 59; and
(c) a VH CDR-H3 comprising the amino acid sequence of SEQ ID NO: 38, 50, or 60; and
(ii) a light chain variable region (VL) that comprises:
(a) a VL CDR-L1 comprising the amino acid sequence of SEQ ID NO: 39, 51, or 61;
(b) a VL CDR-L2 comprising the amino acid sequence of SEQ ID NO: 40, 52, or 62; and
(c) a VL CDR-L3 comprising the amino acid sequence of SEQ ID NO: 41, 53, or 63.

2. The antibody, or antigen-binding fragment thereof, of claim 1, comprising a VH that comprises the amino acid sequence of SEQ ID NO: 42, 54, or 64.

3. The antibody, or antigen-binding fragment thereof, claim 1, comprising a heavy chain constant region (CH) that comprises the amino acid sequence of SEQ ID NO: 46.

4. The antibody, or antigen-binding fragment thereof, claim 1, comprising a VL that comprises the amino acid sequence of SEQ ID NO: 43, 55, or 65.

5. The antibody, or antigen-binding fragment thereof, of claim 1, comprising a light chain constant region (CL) that comprises the amino acid sequence of SEQ ID NO: 47.

6. The antibody, or antigen-binding fragment thereof, of claim 1, comprising a heavy chain that comprises the amino acid residues 23 to 466 of SEQ ID NO: 44.

7. The antibody, or antigen-binding fragment thereof, of claim 1, comprising a light chain that comprises the amino acid residues 23 to 236 of SEQ ID NO: 45.

8. The antibody, or antigen-binding fragment thereof, of claim 1, comprising a heavy chain that comprises the amino acid residues 23 to 466 of SEQ ID NO: 56.

9. The antibody, or antigen-binding fragment thereof, of claim 1, comprising a light chain that comprises the amino acid residues 23 to 236 of SEQ ID NO: 57.

10. The antibody, or antigen-binding fragment thereof, of claim 1, comprising a heavy chain that comprises the amino acid residues 23 to 465 of SEQ ID NO: 66.

11. The antibody, or antigen-binding fragment thereof, of claim 1, comprising a light chain that comprises the amino acid residues 23 to 236 of SEQ ID NO: 67.

12. A pharmaceutical composition comprising the antibody, or antigen binding fragment there of, of claim 1, further comprising a pharmaceutically acceptable excipient.
